(19) United States
(12) Patent Application Publication
(10) Pub. No.: US 2025/0186621 A1
(43) Pub. Date: Jun. 12, 2025

(54) MATERIALS AND METHODS FOR THE TREATMENT OF EIF2B5 MUTATIONS AND DISEASES RESULTING THEREFROM

(71) Applicants: RESEARCH INSTITUTE AT NATIONWIDE CHILDREN'S HOSPITAL, Columbus, OH (US); UNIVERSITY OF UTAH RESEARCH FOUNDATION, Salt Lake City, UT (US)

(72) Inventors: Kevin Flanigan, Columbus, OH (US); Allison Marie Bradbury, Columbus, OH (US); Joshua Bonkowsky, Salt Lake City, UT (US); Nettie Kate Pyne, Galloway, OH (US); Jessica Herstine, Columbus, OH (US)

(21) Appl. No.: 18/837,099

(22) PCT Filed: Mar. 3, 2023
(86) PCT No.: PCT/US2023/063676

§ 371 Date: Aug. 8, 2024

Related U.S. Application Data
(60) Provisional application No. 63/387,223, filed on Dec. 13, 2022.
(60) Provisional application No. 63/316,241, filed on Mar. 3, 2022.

(51) Int. Cl. A61K 48/00; C07K 14/47; C12N 15/86

(52) U.S. Cl. CPC A61K 48/0058; A61K 48/0066; C07K 14/47; C12N 15/86; C12N 2750/14143; C12N 2830/60

(57) ABSTRACT

Provided are gene therapy vectors, such as adeno-associated virus (AAV), designed for treatment of mutations in the Eukaryotic Translation Initiation Factor 2B Subunit Epsilon (EIF2B5) gene. The EIF2B5 gene provides instructions for making one of five subunits of the elF2B protein, specifically the epsilon subunit of this protein. Such mutations are associated with a disease or disorder such as a leukoencephalopathy, a megalencephalic leukoencephalopathy, a leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), AicardinGoutieres syndrome, CLC-2-related disease, oculodentodigital dysplasia, and/or giant axonal neuropathy. Such leukoencephalopathies or leukodystrophies include, but are not limited to, Vanishing White Matter Disease (VWM). The disclosed gene therapy vectors provide a EIF2B5 cDNA to a subject in need which results in expression of a wild type or functional EIF2B5 protein. Also provided is a new promoter, designated gfa1405, which was designed to target astrocytes and neurons. Thus, compositions, nanoparticles, extracellular vesicles, exosomes, or vector comprising the gfa1405 promoter and methods of its use are also provided.

This application contains, as a separate part of the disclosure, a Sequence Listing in computer-readable form which is incorporated by reference in its entirety and identified as follows: 56440_Seqlisting.XML; Size: 70,395 bytes; Created: Feb. 16, 2023.

### FIELD OF THE INVENTION

The disclosure provides gene therapy vectors, such as adeno-associated virus (AAV), designed for treatment of mutations in the gene encoding the Eukaryotic Translation Initiation Factor 2B Subunit Epsilon 5 (EIF2B5) protein. Such mutations in the EIF2B5 gene are associated with a leukoencephalopathy, a megalencephalic leukoencephalopathy, a leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, or vanishing white matter (VWM) disease. The disclosed gene therapy vectors provide a EIF2B5 cDNA or a wild type EIF2B5 cDNA to a subject in need which results in expression of a functional or wild type EIF2B5 protein. The disclosure also provides a novel promoter designed to target expression in astrocytes and/or neurons.

### BACKGROUND

Vanishing White Matter Disease (VWM) is a devastating leukodystrophy (1-3). VWM is caused by autosomal recessive mutations in the five subunit genes of the Eukaryotic Initiation Factor 2B (EIF2B) complex, which is necessary for the first steps of protein translation. There are currently no therapies for VWM disease.

Studies have shown that the majority of VWM mutations are in the EIF2B5 gene, followed by the EIF2B2 gene (1, 4). The exact mechanism of pathology remains unknown, although it is evident that glial cell dysfunction is fundamental to the pathophysiology. Established cell cultures from the brain of an individual with EIF2B5 VWM revealed that healthy oligodendrocytes were readily generated; however, few astrocytes were present. Further, induction of astrocytes was severely diminished, and the few astrocytes generated were irregular. Lesions in vivo also lacked glial fibrillary acidic protein (GFAP)-positive (GFAP+) astrocytes. Further, targeting of elF2B5 with RNA interference severely compromised the induction of GFAP+ astrocytes from normal human glial progenitors (5). There are currently three murine models of elF2B5 VWM (6-8). Impaired maturation of white matter astrocytes preceded onset and paralleled disease severity and progression in two of the models. In coculture, VWM astrocytes secreted factors that inhibited oligodendrocyte maturation, whereas WT astrocytes allowed normal maturation of VWM oligodendrocytes (7). Taken together, these studies from a human patient and murine models demonstrate that astrocytes are central in VWM pathology and constitute potential therapeutic targets.

Advances in adeno-associated virus (AAV) vectors have led to safer and more efficient viral vehicles to deliver therapeutic transgenes in a single injection, and gene therapy is now a favorable therapeutic intervention for monogenic diseases. AAV serotype 9 has become the most widely used vector for neurological indications and has established a safety profile in the clinic. Intrathecal administration of AAV9 permits dissemination of transgenes throughout the nervous system and is currently in trials for the treatment of neuronal ceroid lipofuscinosis 3 (CLN3, NCT03770572), CLN6 (NCT02725580), spinal muscular atrophy (SMA, NCT03381729), and giant axonal neuropathy (GAN, NCT02362438). However, it has been shown across species including mice (9), dogs (10), and non-human primates (11, 12) that AAV9 primarily targets neurons and fails to effectively target glia, a clear therapeutic target for VWM disease and other leukodystrophies. Nonetheless, it is now possible to achieve potent cell-specific tropism by driving transgene expression with select promoters. Notably, GFAP promoter-driven transgene expression has been shown to be highly specific for astrocytes following AAV infusion to the brains of neonatal and adult mice (13).

Because patients with a mutation in their elF2B5 gene suffer from or are risk of suffering from leukoencephalopathy with vanishing white matter, there is an urgent need for new therapeutic options. Moreover, elF2B5 mutations have been implicated in other diseases or disorders, such as megalencephalic leukoencephalopathy, leukodystrophy, stroke, migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, and giant axonal neuropathy. The disclosure provides a replacement elF2B5 nucleic acid and EIF2B2 gene replacement as a feasible therapeutic strategy to treat a mutation(s) in the EIF2B2 gene and treat, prevent, or ameliorate a leukodystrophy, leukoencephalopathy and/or VWM disease resulting from such mutation(s). The disclosure thus provides nucleic acids, nanoparticles, extracellular vesicles, exosomes, vectors, recombinant AAV particles, and compositions comprising the elF2B5 gene for treating elF2B5 mutations. The disclosed products, methods and uses provide a feasible approach for robust and long-term expression of the elF2B5 protein, or a functional elF2B5, in cells of the human brain.

### SUMMARY

Provided herein are products, methods, and uses for treating mutations in the gene encoding □the Eukaryotic Translation Initiation Factor 2B Subunit Epsilon 5 (EIF2B5)□ and in treating, ameliorating, delaying the progression of, and/or preventing diseases resulting from mutations in the EIF2B5 gene.

The disclosure provides a nucleic acid comprising a polynucleotide comprising (a) one or more regulatory control element(s); and (b) a EIF2B5 cDNA sequence. In some aspects, the EIF2B5 cDNA comprises (a) a nucleotide sequence having at least 80% sequence identity to SEQ ID NO: 1; (b) the nucleotide sequence set forth in SEQ ID NO: 1; or (c) a nucleotide sequence encoding EIF2B5 comprising the amino acid sequence set forth in SEQ ID NO: 2.

In some aspects, the one or more regulatory control element(s) is a CAG promoter, a gfaABC1D promoter, a GFAP promoter, or a functional fragment of any of the CAG promoter, the gfaABC1D promoter, or the GFAP promoter. In some aspects, the regulatory control element comprises (a) a nucleotide sequence having at least 80% sequence identity to SEQ ID NO: 3, 4, 5, or 15; or (b) the nucleotide sequence set forth in SEQ ID NO: 3, 4, 5, or 15.

In some aspects, the nucleic acid further comprises an SV40 intron and a post-transcriptional polyadenylation (polyA) sequence. In some aspects, the nucleic acid comprises (a) a nucleotide sequence having at least 80% sequence identity to any one of SEQ ID NOs: 6-8 and 16; or (b) the nucleotide sequence set forth in any one of SEQ ID NOs: 6-8 and 16.

In some aspects, the nucleic acid further comprises an inverted terminal repeat sequence. In some aspects, the nucleic acid comprises (a) a nucleotide sequence having at least 80% sequence identity to any one of SEQ ID NOs: 9-11 and 17; or (b) the nucleotide sequence set forth in any one of SEQ ID NOs: 9-11 and 17.

In some aspects, the nucleic acid further comprises additional sequence of the AAV genome. In some aspects, the nucleic acid comprises (a) a nucleotide sequence having at least 80% sequence identity to any one of SEQ ID NOs: 12-14 and 18; or (b) the nucleotide sequence set forth in any one of SEQ ID NOs: 12-14 and 18.

In some aspects, a nucleic acid of the disclosure comprises (a) a nucleotide sequence having at least 80% sequence identity to any one of SEQ ID NOs: 12-14 and 18; or (b) a nucleotide sequence having at least 80% sequence identity to any one of the nucleotide sequences of the (1) the 5′ ITR, (2) the CAG, gfaABC (1), GFAP, or gfa1405 promoter, (3) the SV40 intron, (4) the elF2B5 ORF, (5) the polyA, (6) the F1 Origin, (7) the kanamycin resistance gene, and the (8) the pMB1 Origin shown in the sequences set forth in any one of SEQ ID NOs: 12-14 and 18 and in FIGS. 12-14 and 21.

The disclosure provides a nanoparticle, extracellular vesicle, exosome, or vector comprising any of the nucleic acids of the disclosure or a combination of any one or more thereof. In some aspects, the vector is a viral vector. In some aspects, the viral vector is an adeno-associated virus (AAV), adenovirus, lentivirus, retrovirus, poxvirus, baculovirus, herpes simplex virus, vaccinia virus, or a synthetic virus. In some aspects, the viral vector is an AAV. In some aspects, the AAV comprises rep and cap genes. In some aspects, the AAV lacks rep and cap genes. In some aspects, the AAV is a recombinant AAV (rAAV), a self-complementary recombinant AAV (scAAV), or a single-stranded recombinant AAV (ssAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh. 10, MyoAAV 1A, AAVMYO, or AAV-B1, AAV2/1, AAV2/8, AAV2/9, or any of their derivatives. In some aspects, the AAV is AAV9. In some aspects, the ITR sequences present in the AAV are from AAV2. In some particular aspects, the AAV is an ssAAV or an ssrAAV.

The disclosure provides an rAAV particle comprising any of the AAV of the disclosure.

The disclosure provides a composition comprising any one or more of the nucleic acids, nanoparticles, extracellular vesicles, exosomes, vectors, viral vectors, or rAAV particles of the disclosure, and a pharmaceutically acceptable carrier. In some aspects, the composition is formulated for intrathecal, intracerebroventricular, intracerebral, intravenous, intracisternal (or intra cisterna magna (ICM), or aerosol delivery.

The disclosure provides a method of increasing the expression of a EIF2B5 gene or EIF2B5 protein in a cell comprising contacting the cell with any one or more of the nucleic acids, nanoparticles, extracellular vesicles, exosomes, vectors, viral vectors, rAAV particles, or compositions of the disclosure. In some aspects, the cell is an astrocyte, a neuron, or a glial cell. In some aspects, the cell is an astrocyte. In some aspects, the cell is a human cell. In some aspects, the cell is in a human subject.

The disclosure provides a method of treating a subject comprising a EIF2BB5 gene mutation comprising administering to the subject an effective amount of any one or more of the nucleic acids, nanoparticles, extracellular vesicles, exosomes, vectors, viral vectors, rAAV particles, or compositions of the disclosure. In some aspects, the subject is a human subject. In some aspects, the EIF2BB5 gene mutation causes a subject to suffer from or be at risk of suffering from a leukoencephalopathy, a megalencephalic leukoencephalopathy, a leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, or vanishing white matter (VWM) disease. In some aspects, the method further comprises a combination therapy. In some aspects, the method further comprises administering any one or more of a corticosteroid, rituximab, and rapamycin to the subject. In some aspects, the nucleic acid, nanoparticle, extracellular vesicle, exosome, vector, rAAV particle, or composition is administered by intrathecal, intracerebroventricular, intracerebral, intravenous, intracisternal (or intra cisterna magna (ICM), or aerosol delivery.

The disclosure provides a use of any one or more of the nucleic acids, nanoparticles, extracellular vesicles, exosomes, vectors, viral vectors, rAAV particles, or compositions of the disclosure for the preparation of a medicament for increasing expression of the EIF2B5 gene or protein in a cell. In some aspects, the cell is in a human subject. In some aspects, the subject suffers from a EIF2B5 mutation, and in some aspects this mutation is associated with a leukoencephalopathy, a megalencephalic leukoencephalopathy, a leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, or vanishing white matter (VWM) disease. In some specific aspects, the leukoencephalopathy or leukodystrophy is Vanishing White Matter Disease (VWM). In some aspects, the medicament is administered in combination with another medicament. In some aspects, the medicament is administered with any one or more of a corticosteroid, rituximab, and rapamycin. In some aspects, the medicament is formulated for intrathecal, intracerebroventricular, intracerebral, intravenous, intracisternal (or intra cisterna magna (ICM), or aerosol delivery.

The disclosure provides a use of any one or more of the nucleic acids, nanoparticles, extracellular vesicles, exosomes, vectors, viral vectors, rAAV particles, or compositions of the disclosure in treating a subject comprising a mutant EIF2B5 gene. In some aspects, the cell is in a human subject. In some aspects, the subject suffers from a EIF2B5 mutation, a leukoencephalopathy, or a leukodystrophy. In some aspects, the leukoencephalopathy or leukodystrophy is Vanishing White Matter Disease (VWM). In some aspects, the medicament is administered in combination with another medicament. In some aspects, the medicament is administered with any one or more of a corticosteroid, rituximab, and rapamycin. In some aspects, the medicament is formulated for intrathecal, intracerebroventricular, intracerebral, intravenous, or aerosol delivery.

The disclosure provides a composition for treating a EIF2B5 gene mutation, a leukoencephalopathy, a megalencephalic leukoencephalopathy, a leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, or vanishing white matter (VWM) disease in a subject, wherein the composition comprises any one or more of the nucleic acids, nanoparticles, extracellular vesicles, exosomes, vectors, viral vectors, rAAV particles, or compositions of the disclosure. In some aspects, the subject is a human subject. In some aspects, the leukoencephalopathy or leukodystrophy is Vanishing White Matter Disease (VWM).

The disclosure provides any one or more of the nucleic acids, nanoparticles, extracellular vesicles, exosomes, vectors, viral vectors, rAAV particles, compositions, methods, uses, or medicament as described herein. In some aspects, such nucleic acid(s), nanoparticle(s), extracellular vesicle(s), exosome(s), vector(s), viral vector(s), composition(s), or medicament(s) is formulated for intrathecal (direct injection into the CSF), intravenous injection into the blood stream, intracerebral injection, intracerebroventricular injection, intracisternal (or intra cisterna magna (ICM), or for aerosol delivery or administration.

The disclosure also provides a nucleic acid comprising a novel gfa1405 promoter comprising (a) a nucleotide sequence having at least 80% sequence identity to SEQ ID NO: 15; or (b) the nucleotide sequence set forth in SEQ ID NO: 15. In some aspects, the nucleic acid further comprises an inverted terminal repeat sequence. Thus, in some aspects, the nucleic acid comprises (a) a nucleotide sequence having at least 80% sequence identity to SEQ ID NO: 17 or 18; or (b) the nucleotide sequence set forth in SEQ ID NO: 17 or 18.

The disclosure provides a nanoparticle, extracellular vesicle, exosome, or vector comprising such nucleic acid or a combination of any one or more thereof. In some aspects, the vector is a viral vector. In some aspects, the viral vector is an adeno-associated virus (AAV), adenovirus, lentivirus, retrovirus, poxvirus, baculovirus, herpes simplex virus, vaccinia virus, or a synthetic virus. In some aspects, the viral vector is an AAV. In some aspects, the AAV lacks rep and cap genes. In some aspects, the AAV is a recombinant AAV (rAAV), a self-complementary recombinant AAV (scAAV), or a single-stranded recombinant AAV (ssAAV). In some particular aspects, the AAV is an ssAAV or an ssrAAV. In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh. 10, MyoAAV 1A, AAVMYO, or AAV-B1, AAV2/1, AAV2/8, AAV2/9, or any of their derivatives. In some aspects, the AAV is AAV9.

The disclosure provides an rAAV particle comprising any of the AAV of the disclosure.

The disclosure provides a composition comprising any one or more of the nucleic acids, nanoparticles, extracellular vesicles, exosomes, vectors, viral vectors, or rAAV particles, and a pharmaceutically acceptable carrier. In some aspects, the composition is formulated for intrathecal, intracerebroventricular, intracerebral, intravenous, intracisternal, or aerosol delivery.

The disclosure provides a method of increasing the expression of a gene or a protein in a cell comprising contacting the cell with any one or more of the nucleic acids, nanoparticles, extracellular vesicles, exosomes, vectors, viral vectors, or rAAV particles, or compositions of the disclosure. In some aspects, the cell is an astrocyte or a neuron. In some aspects, the cell is a human cell. In some aspects, the cell is in a human subject.

The disclosure provides a method of treating a subject comprising a mutation in a gene normally expressed in an astrocyte or neuron of the subject, the method comprising administering to the subject an effective amount of any one or more of the nucleic acids, nanoparticles, extracellular vesicles, exosomes, vectors, viral vectors, or rAAV particles, or compositions of the disclosure. In some aspects, the subject is a human subject. In some aspects, the gene mutation causes a subject to suffer from or be at risk of suffering from an astrocyte or neuronal disorder or disease. In some aspects, the disorder or disease is a leukoencephalopathy or leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, or a megalencephalic leukoencephalopathy. In some aspects, the leukoencephalopathy or leukodystrophy is Vanishing White Matter Disease (VWM). In some aspects, the methods of the disclosure further comprise administering any one or more of a corticosteroid, rituximab, and rapamycin to the subject. In some aspects, the nucleic acid, nanoparticle, extracellular vesicle, exosome, vector, rAAV particle, or composition is administered by intrathecal, intracerebroventricular, intracerebral, intravenous, intracisternal (or intra cisterna magna (ICM), or aerosol delivery.

The disclosure provides uses of any one or more of the nucleic acids, nanoparticles, extracellular vesicles, exosomes, vectors, viral vectors, or rAAV particles, or compositions of the disclosure for the preparation of a medicament for increasing expression of a gene or protein in a cell. In some aspects, the cell is in a human subject.

The disclosure provides uses of one or more of the nucleic acids, nanoparticles, extracellular vesicles, exosomes, vectors, viral vectors, or rAAV particles, or compositions of the disclosure for treating a subject comprising a mutant gene. In some aspects, the subject is a human subject. In some aspects, the subject suffers from a gene mutation or a disorder or disease affecting the central nervous system and/or brain. In some aspects, the subject suffers from a gene mutation or a disorder or disease affecting astrocytes or neurons of the brain. In some aspects, the subject suffers from a leukoencephalopathy or leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, or a megalencephalic leukoencephalopathy. In some aspects, the leukoencephalopathy or leukodystrophy is Vanishing White Matter Disease (VWM). In some aspects, the medicament is administered with any one or more of a corticosteroid, rituximab, and rapamycin. In some aspects, the medicament is formulated for intrathecal, intracerebroventricular, intracerebral, intravenous, intracisternal (or intra cisterna magna (ICM), or aerosol delivery.

The disclosure provides a composition for treating a gene mutation, or a disease or disorder in the central nervous system and/or brain of a subject, wherein the composition comprises one or more of the nucleic acids, nanoparticles, extracellular vesicles, exosomes, vectors, viral vectors, or rAAV particles, or compositions of the disclosure. In some aspects, the subject is a human subject. In some aspects, the subject suffers from a gene mutation or a disorder or disease affecting the central nervous system and/or brain. In some aspects, the subject suffers from a gene mutation or a disorder or disease affecting astrocytes or neurons of the brain. In some aspects, the subject suffers from a leukoencephalopathy or leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, and/or a megalencephalic leukoencephalopathy. In some aspects, the leukoencephalopathy or leukodystrophy is Vanishing White Matter Disease (VWM).

The disclosure provides one or more of the nucleic acids, nanoparticles, extracellular vesicles, exosomes, vectors, viral vectors, rAAV particles, compositions, or medicaments, wherein the nucleic acids, nanoparticles, extracellular vesicles, exosomes, vectors, viral vectors, or rAAV particles, compositions or medicaments is/are formulated for intrathecal injection into the cerebrospinal fluid (CSF), intravenous injection into the blood stream, intracerebral injection, intracerebroventricular injection, intracisternal injection, or for aerosol administration.

Other features and advantages of the disclosure will become apparent from the following description of the drawings and the detailed description. It should be understood, however, that the drawings, detailed description, and the examples, while indicating embodiments of the disclosed subject matter, are given by way of illustration only, because various changes and modifications within the spirit and scope of the disclosure will become apparent from the drawing, detailed description, and the examples.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the four AAV constructs that were designed. These constructs include promoters targeting astrocytes: full length GFAP and the truncated version gfaABC(1)D, the novel gfaABCD1405 (or gfa1405) promoter, as well as a ubiquitous promoter comprised of a CBA promoter and CMV enhancer, also referred to as CAG. The other key components of the vectors include AAV9 capsid for efficient targeting of the CNS, AAV2 inverted terminal repeats (ITRs) creating a single-stranded construct with a larger packaging capacity, an SV40 intron, the human elF2B5 coding sequence, and a post-transcriptional polyadenylation (polyA) sequence.

FIG. 2 shows the in vitro expression of GFP in transfected HEK293T cells for each of the 3 eGFP constructs: AAV9-CAG-GFP, AAV9-gfaABC(1)D-GFP, and AAV9-CBA-GFP vectors. Specifically, HEK293 cells were transfected by lipofectamine and 72 hours post-transfection cells were harvested for mRNA expression analysis of eGFP. Although HEK cells were not the ideal cell model because astrocytes are the target cells for these constructs, HEK293 cells allowed for rapid in vitro analysis to determine if the plasmids expressed the gene of interest. At 72 hours post-transfection GFP positive cells were detectable by microscopy (left panel). Additionally, quantitative PCR of mRNA expression on cell pellets showed increased expression, CAG ˜3500-fold, GFAP ˜1200-fold, and GfaABC(1)D˜900-fold over untransfected control cells (right panel). These results showed expression of the transgene driven by the various promoters in cells in vitro.

FIG. 3 shows astrocytes derived from fibroblasts stained with glial fibrillary acidic protein (GFAP), a marker for astrocytes. The astrocytes were established as a relevant in vitro system to test gene therapy constructs of the disclosure. The astrocytes were derived from fibroblasts cultured from a skin biopsy of a patient with confirmed elF2B5 VMD disease. This provides an in vitro system of VWM astrocytes to study disease mechanism and test therapies.

FIG. 4 shows results of experiments with a wild-type mouse injected with AAV9-CAG-GFP at postnatal day 1 (PND1) and sacrificed 28 days post-injection. A sagittal section of the brain shows distribution of GFP (green). DAPI (blue) indicates nuclei. Insets 1-6 show higher magnification of the associated voxels on the sagittal section.

FIG. 5 shows results of experiments with a wild-type mouse injected with AAV9-CAG-GFP at PND1 and sacrificed 28 days post-injection. A sagittal section of the brain shows distribution of GFP (green). DAPI (blue) indicates nuclei. Insets show higher magnification of GFP positive glial and neuronal cells.

FIG. 6 shows results of experiments with four wild-type mice injected with AAV9-CAG-GFP at PND1 and sacrificed 28 days post-injection. A sagittal section of the each of the mouse brains shows distribution of GFP (green). Injection of neonatal wildtype mice with AAV9-gfaABC(1)D-GFP resulted in greater distribution in the white matter and more global distribution throughout the neuroaxis as compared to AAV9-CBA-GFP. Particularly notable is the increased GFP expression in the caudal brain regions including the white matter regions of the cerebellum in box 2. Further, expression was primarily in astrocytes, as indicated by the morphology in box 4.

FIG. 7 shows results of an experiment with a wild-type mouse injected with AAV9-gfaABC(1)D-GFP at PND1 and sacrificed 28 days post-injection. A sagittal section of the brain shows distribution of GFP (green). Insets 1-3 show higher magnification of the associated voxels on the sagittal section. Inset 4 shows higher magnification of inset 3 to demonstrate astrocytic morphology.

FIG. 8 shows results of experiments with three wild-type mice injected with AAV9-gfaABC(1)D-GFP at PND1 and sacrificed 28 days post-injection. A sagittal section of the brain shows distribution of GFP (green).

FIG. 9 shows results of an experiment with a wild-type mouse injected with AAV9-GFAP-GFP at PND1 and sacrificed 28 days post-injection. A sagittal section of the brain shows distribution of GFP (green). Inset shows higher magnification of cerebellar white matter, indicated by the red arrow. Injection of neonatal wild-type mice with AAV9-GFAP-GFP, to date, has resulted in more widespread distribution and greater expression as compared to both AAV9-gfaABC(1)D-GFP and AAV9-CBA-GFP vectors. Expression has remained retained to astrocytes.

FIG. 10 shows results of experiments with four wild-type mice injected with AAV9-GFAP-GFP at PND1 and sacrificed 28 days post-injection. A sagittal section of the brain shows distribution of GFP (green). Similar biodistribution patterns were seen between the injected mice.

FIG. 11 shows results of an experiment with a wild-type mouse injected with AAV9-GFAP-GFP at PND1 and sacrificed 28 days post-injection. Co-labeling shows GFP (green) expression colocalizing with astrocyte marker (GFAP, purple) and not neuronal marker (NeuN, red). GFP expression in astrocytes was confirmed by co-labeling with an antibody specific for astrocytes (GFAP, purple) and neurons (NeuN, red). GFP expression after treatment with AAV9-GFAP-GFP (shown) and AAV9-gfaABC(1)D-GFP (not shown) was clearly present in astrocytes. Thus, the expression was confirmed to occur primarily in astrocytes, which are the target cells.

FIG. 12 provides the annotated sequence (SEQ ID NO: 12) for AAV.CAG.elF2B5.

FIG. 13 provides the annotated sequence (SEQ ID NO: 13) for AAV.gfaABC1D.elF2B5.

FIG. 14 provides the annotated sequence (SEQ ID NO: 14) for AAV.GFAP.elF2B5.

FIG. 15 provides the sequence (SEQ ID NO: 15) for the novel gfa1405 promoter (also called the gfaABCD1405 promoter).

FIG. 16 provides the annotated sequence (SEQ ID NO: 17) for the full ITR-gfa1405-elF2B5-polyA-ITR sequence.

FIG. 17 shows the design and components of the novel gfa1405 promoter.

FIG. 18 shows the design and components of the full ITR-gfa1405-elF2B5-polyA-ITR sequence.

FIG. 19 shows the plasmid map of pAAV.gfa1405.EIF2B5 (7086 bp of SEQ ID NO: 18).

FIG. 20A-B shows that GFP expression in HEK293T cells could be achieved by both ubiquitous and astrocyte-specific promoters, including the novel gfa1405 promoter. FIG. 20A shows GFP expression in HEK293T cells 72 hours post-transfection with four various promoters discussed herein, i.e., CAG, GFAP, gfaABC(1)D, and gfaABCD1405 (or gfa1405).

FIG. 20B shows results of the quantification of percent CAG expression after a western blot for GFP expression was carried out in triplicate for all constructs in comparison to expression of the house keeping protein, GAPDH. GFP protein expression was greater for the gfaABCD1405 (gfa1405) construct than that of the GFAP and gfaABC(1)D constructs, but not as great as that of the ubiquitous promoter CAG construct.

FIG. 21 provides the annotated sequence (SEQ ID NO: 18) for AAV.gfa1405.elF2B5 (7086 bp).

FIG. 22 shows how gene therapy constructs were designed to evaluate cell-specific expression through different AAV reporter constructs.

FIG. 23 shows GFP expression in the mouse brain 4 weeks (top row) and 8 weeks (bottom row) after ICV delivery of AAV9-CAG-GFP by intracerebroventricular (ICV) on PND 1. Green staining represents GFP, blue represents DAPI. This data is in furtherance to data shown in FIGS. 4-6.

FIG. 24 shows GFP expression in the mouse brain 4 weeks (top row) and 8 weeks (bottom row) after ICV delivery of AAV9-CAG-GFP by intracerebroventricular (ICV) on PND 1. Green staining represents GFP, blue represents DAPI. This data is in furtherance to data shown in FIGS. 9-11.

FIG. 25 shows GFP expression in the mouse brain 4 weeks (top row) and 8 weeks (bottom row) weeks after ICV delivery of AAV9-gfABC(1)D-GFP by intracerebroventricular (ICV) on PND 1. Green staining represents GFP, blue represents DAPI. This data is in furtherance to data shown in FIGS. 7 and 8.

FIG. 26 shows IF quantification of GFP constructs, i.e., percentage GFP-positive area in in the total mouse brain and GFP intensity in the GFP positive areas 4 and 8 weeks after ICV delivery of AAV9-GFAP-GFP, AAV9-gfABC(1)D-GFP, AAV9-CAG-GFP by intracerebroventricular (ICV) on PND 1.

FIG. 27 shows GFP expression in whole brains 4 weeks after ICV delivery of AAV9-GFAP-GFP, AAV9-gfABC(1)D-GFP, AAV9-CAG-GFP. The bottom panels show Western blot analysis of GFP protein as compared to house keeping gene, GAPDH 4 weeks and 8 weeks after treatment.

FIG. 28 shows the colocalization of GFP with astrocytic (GFAP) and neuronal (NeuN) markers demonstrating expression limited to astrocytes with GFAP and gfaABC(1)D promoters while CAG resulted in GFP expression in neurons and astrocytes.

FIG. 29 shows GFP expression in the mouse brain 4 weeks after ICV delivery of AAV9-gfa1405-GFP by intracerebroventricular (ICV) on PND 1.

FIG. 30 shows various methods used to assess efficacy of the cell-specific gene therapy constructs comprising the EIF2B5 transgene in treating the model of VWM disease (VWM mice)

FIG. 31 shows therapeutic efficacy in the treatment of VWM mice. Rotarod data in two different mouse models of EIF2B5, Eif2b5198M at Day 50 and Eif2b5R191H at 6 months. Both models show normalized latency (Eif2b5198M) or RPM at fall (Eif2b5R191H) after treatment with AAV9-gfABC(1)D-GFP. Lesser improvement was seen with AAV9-GFAP-GFP and AAV9-CAG-GFP promoters.

### DETAILED DESCRIPTION

The disclosure provides Eukaryotic Translation Initiation Factor 2B Subunit Epsilon 5 (EIF2B5) gene replacement as a feasible therapeutic strategy to treat a mutation(s) in the gene that encodes EIF2B5 and, as a result, treat, ameliorate, delay the progression of, or prevent a disease or disorder resulting from the mutation including, but not limited to, a leukoencephalopathy, a megalencephalic leukoencephalopathy, a leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, or vanishing white matter (VWM) disease. The disclosed products, methods and uses provide a feasible approach for robust and long-term expression of the EIF2B5 gene in human neurons and glial cells in the treatment of a leukoencephalopathy, a megalencephalic leukoencephalopathy, a leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, or vanishing white matter (VWM) disease.

Leukodystrophies are a heterogeneous group of disorders with highly variable clinical manifestations and pathologic mechanisms. They are loosely grouped together, usually based on the initial findings of white matter abnormalities in the central nervous system (CNS), historically based on gross pathology, and now often based on neuroimaging. There has never been, however, a formal definition or classification for this group of disorders. The term leukodystrophy technically refers to disorders with wasting (dystrophy) of the brain's white matter (leuko) and is traditionally reserved for heritable disorders, however there is lack of consensus on how this term should be applied.

Further complicating the definition of leukodystrophies, the related but distinct term ileukoencephalopathy exists in the literature. This term has characteristically been applied to disorders seen in the context of toxic, acquired vascular or infectious insults, as well as inherited disorders. In addition, disparate terms, such as hypomyelination, demyelination and dysmyelination are in use, and are a source of confusion.

VWM is a type of leukodystrophy, which is caused by mutations in one of the five genes EIF2B1, EIF2B2, EIF2B3, EIF2B4 and EIF2B5 that encode the five subunits of a protein called eukaryotic initiation factor 2B (elF2B). This protein is necessary for the production of all other proteins in the body and for the regulating the rate of protein production, especially the decrease in protein synthesis during stress conditions, such as fever and infection. It is so important that no one can live when any of these genes are completely non-functional or absent. VWM is caused by small changes in these genes that reduce the function of elF2B, and specific cells in the brain are particularly vulnerable to this loss of function. The reduction in function becomes a particular problem during episodes of fever, infection, or head trauma, and deterioration accelerates following such episodes.

VWM is inherited in an autosomal recessive manner. Other clinical names for VWM include, but are not limited to, childhood ataxia with diffuse CNS hypomyelination (CACH), Vanishing White Matter leukodystrophy, Cree leukoencephalopathy, leukodystrophy with ovarian failure, ovarioleukodystrophy, and elF2B-related disorders. VWM is a leukodystrophy, a neurodegenerative white matter disorder that most commonly occurs in children. Clinically, it presents with ataxia, spasticity, neurological decline, and seizures which lead to premature death. Currently there are no treatments for VWM. VWM is caused by autosomal recessive, loss of function mutations in the subunits of eukaryotic initiation factor 2B (EIF2B), with pathologic variants in EIF2B5 being the most common. Due to VWM's monogenic nature, it is a good candidate for adeno-associated virus (AAV)-mediated gene replacement therapy. VWM pathology suggests that astrocytes are a critical target for therapy, as their differentiation, morphology, and function is impaired, thus mediating disease progression. Therefore, the disclosure provides gene replacement constructs to compare astrocyte-specific or ubiquitous expression of the transgene.

The EIF2B1 gene provides instructions for making one of five parts of a protein called elF2B, specifically the alpha subunit of this protein. The elF2B protein helps regulate overall protein production (synthesis) in the cell by interacting with another protein, elF2. The elF2 protein is called an initiation factor because it is involved in starting (initiating) protein synthesis. Under some conditions, elF2B increases protein synthesis by helping to recycle molecules called GTP, which carry energy to the initiation factor. Under other conditions, it slows protein synthesis by binding tightly to the initiation factor, which converts the elF2B protein into an inactive form and prevents recycling of GTP. Proper regulation of protein synthesis is vital for ensuring that the correct levels of protein are available for the cell to cope with changing conditions. For example, cells must synthesize protein much faster if they are multiplying than if they are in a resting state.

The EIF2B1 gene (HGNC: 3257 NCBI Entrez Gene: 1967 Ensembl: ENSG00000111361 OMIM®: 606686 UniProtKB/Swiss-Prot: Q14232) encodes one of five subunits of eukaryotic translation initiation factor 2B (EIF2B), a GTP exchange factor for eukaryotic initiation factor 2 and an essential regulator for protein synthesis. Mutations in this gene and the genes encoding other EIF2B subunits have been associated with leukoencephalopathy with vanishing white matter.

The disclosure focuses on providing a EIF2B replacement gene or □transgene□in order to express normal or functionally active EIF2B protein. To accomplish this, specifically designed EIF2B replacement genes or □transgenes□are provided.

In some aspects, the nucleic acid of the EIF2B replacement gene comprises the nucleotide sequence set forth in SEQ ID NO: 1 or a codon-optimized variant of the nucleotide sequence set forth in SEQ ID NO: 1. In various aspects, the nucleic acid is an isoform or variant of the nucleotide sequence nucleotide sequence set forth in set forth in SEQ ID NO: 1. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in SEQ ID NO: 1.

In some aspects, the polypeptide is a EIF2B polypeptide comprising the amino acid sequence set forth in SEQ ID NO: 2. In various aspects, the polypeptide is an isoform or variant of the EIF2B polypeptide comprising the amino acid sequence set forth in SEQ ID NO: 2. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the amino acid sequence set forth in SEQ ID NO: 2.

In some aspects, the transgene polynucleotide sequence is operatively linked to transcriptional control elements (including, but not limited to, promoters, enhancers and/or polyadenylation signal sequences) that are functional in target cells. In some aspects, the promoter is selected to target astrocytes. In some aspects, the promoter is a CAG promoter, the GFAP promoter, a gfaABC1D promoter, or a gfa1405 (also called gfaABCD1405) promoter.

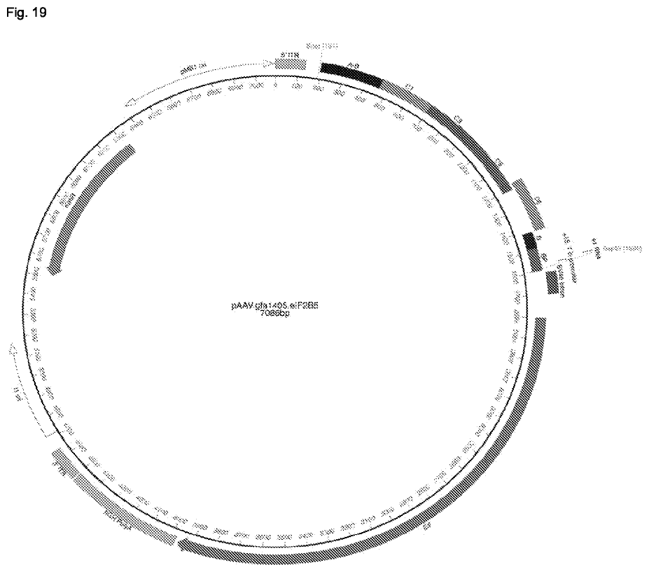

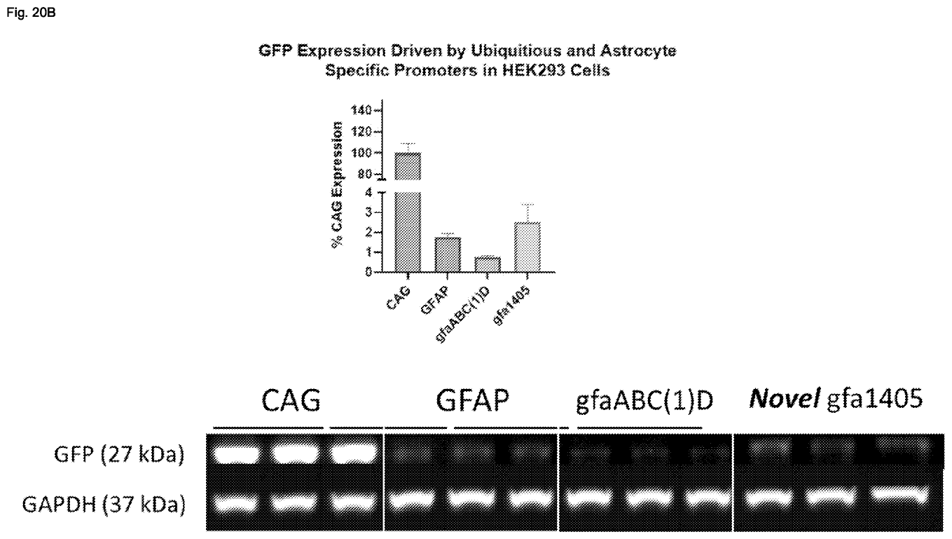

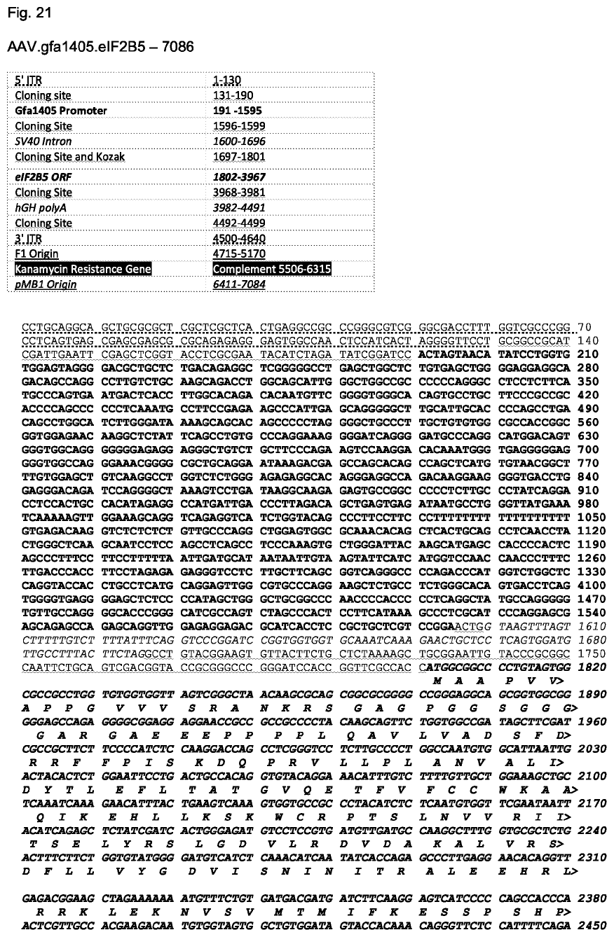

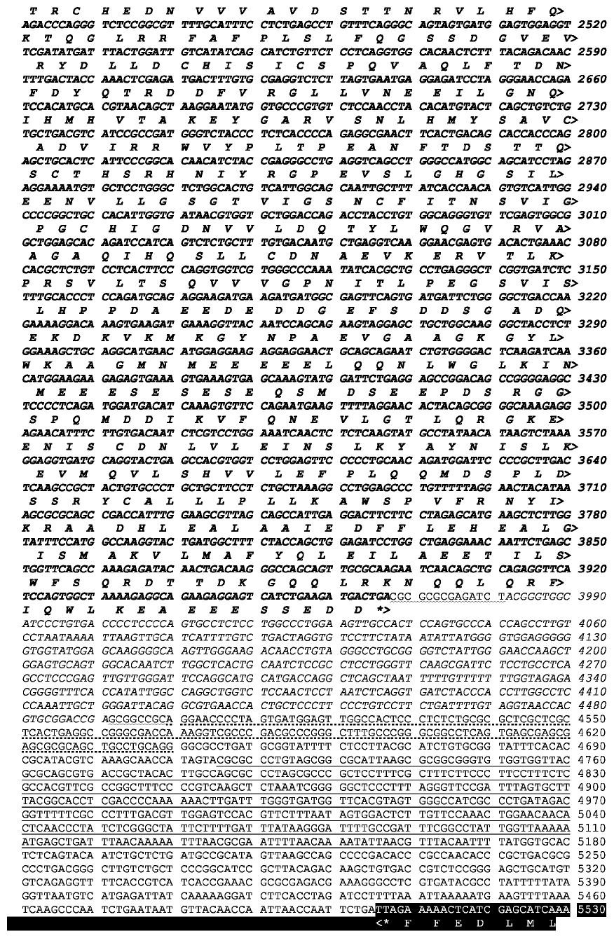

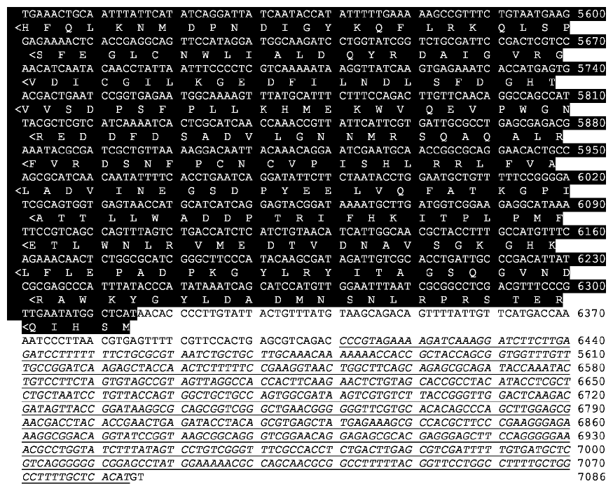

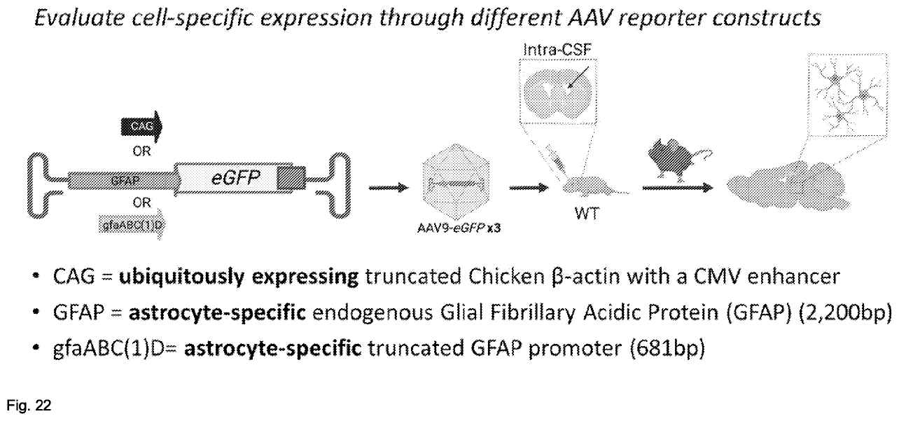

The gfa1405 or gfaABCD1405 promoter is a novel promoter, first described herein (including in more detail in Example 5), and comprises the nucleotide sequence of SEQ ID NO: 15. The gfa1405 promoter was designed to specifically target astrocytes and neurons and can be used to express any gene desired to be expressed in astrocytes or neurons. In some aspects, therefore, the gfa1405 promoter is designed and used to express elF2B5. FIG. 19 provides a schematic drawing of the plasmid, pAAV.gfa1405.elF2B5, which comprises 7086 bp (SEQ ID NO: 18) comprising the gfa1405 promoter, i.e., SEQ ID NO: 15, with the elF2B5 transgene sequence in the AAV vector. FIG. 21 provides a detailed description of the 7086 bp sequences of SEQ ID NO: 18.

The CAG promoter is a ubiquitous promoter which targets neurons and astrocytes. The gfaABC1D promoter and the GFAP promoter drive transgene expression primarily toward astrocytes.

In some aspects, therefore, the promoter is a CAG promoter, a gfaABC1D promoter, a GFAP promoter, or the novel gfa1405 promoter. Although the CAG promoter is commonly referred to only as the “CAG promoter”, it is not a promoter in a strict sense, as it comprises both a promoter and an enhancer. The CAG promoter of SEQ ID NO: 3 comprises a CMV enhancer (nucleotides 1-306 of SEQ ID NO: 3) and a CBA promoter (nucleotides 307-581 of SEQ ID NO: 3).

In some aspects, the CAG promoter comprises the nucleotide sequence set forth in SEQ ID NO: 3. In some aspects, the gfaABC1D promoter comprises the nucleotide sequence set forth in SEQ ID NO: 4. In some aspects, the GFAP promoter comprises the nucleotide sequence set forth in SEQ ID NO: 5. In some aspects, the gfa1405 promoter comprises the nucleotide sequence set forth in SEQ ID NO: 15. Thus, in some exemplary aspects, the nucleic acid comprises a promoter comprising the nucleotide sequence set forth in any one of SEQ ID NOs: 3-5 and 15. In various aspects, the nucleic acid is an isoform or variant of a nucleic acid comprising the nucleotide sequence set forth in in any one of SEQ ID NOs: 3-5. In some aspects, the isoform or variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 3-5 and 15.

TABLE 1
Sequences of the human elF2B5 gene,
elF2B5 protein, and brain-cell
specific promoters.

| | | SEQ |
|---|---|---|
| Sequence | | ID |
| Name | Sequence (5′-3′) | NO: |
| elF2B5 | ATGGCGGCCC CTGTAGTGGC GCCGCCTGGT | 1 |
| gene | GTGGTGGTTA GTCGGGCTAA CAAGCGCAGC | |
| | GGCGCGGGGC CGGGAGGCAG CGGTGGCGGG | |
| | GGAGCCAGAG GGGCGGAGGA GGAACCGCCG | |
| | CCGCCCCTAC AAGCAGTTCT GGTGGCCGAT | |
| | AGCTTCGATC GCCGCTTCTT CCCCATCTCC | |
| | AAGGACCAGC CTCGGGTCCT CTTGCCCCTG | |
| | GCCAATGTGG CATTAATTGA CTACACTCTG | |
| | GAATTCCTGA CTGCCACAGG TGTACAGGAA | |
| | ACATTTGTCT TTTGTTGCTG GAAAGCTGCT | |
| | CAAATCAAAG AACATTTACT GAAGTCAAAG | |
| | TGGTGCCGCC CTACATCTCT CAATGTGGTT | |
| | CGAATAATTA CATCAGAGCT CTATCGATCA | |
| | CTGGGAGATG TCCTCCGTGA TGTTGATGCC | |
| | AAGGCTTTGG TGCGCTCTGA CTTTCTTCTG | |
| | GTGTATGGGG ATGTCATCTC AAACATCAAT | |
| | ATCACCAGAG CCCTTGAGGA ACACAGGTTG | |
| | AGACGGAAGC TAGAAAAAAA TGTTTCTGTG | |
| | ATGACGATGA TCTTCAAGGA GTCATCCCCC | |
| | AGCCACCCAA CTCGTTGCCA CGAAGACAAT | |
| | GTGGTAGTGG CTGTGGATAG TACCACAAAC | |
| | AGGGTTCTCC ATTTTCAGAA GACCCAGGGT | |
| | CTCCGGCGTT TTGCATTTCC TCTGAGCCTG | |
| | TTTCAGGGCA GTAGTGATGG AGTGGAGGTT | |
| | CGATATGATT TACTGGATTG TCATATCAGC | |
| | ATCTGTTCTC CTCAGGTGGC ACAACTCTTT | |
| | ACAGACAACT TTGACTACCA AACTCGAGAT | |
| | GACTTTGTGC GAGGTCTCTT AGTGAATGAG | |
| | GAGATCCTAG GGAACCAGAT CCACATGCAC | |
| | GTAACAGCTA AGGAATATGG TGCCCGTGTC | |
| | TCCAACCTAC ACATGTACTC AGCTGTCTGT | |
| | GCTGACGTCA TCCGCCGATG GGTCTACCCT | |
| | CTCACCCCAG AGGCGAACTT CACTGACAGC | |
| | ACCACCCAGA GCTGCACTCA TTCCCGGCAC | |
| | AACATCTACC GAGGGCCTGA GGTCAGCCTG | |
| | GGCCATGGCA GCATCCTAGA GGAAAATGTG | |
| | CTCCTGGGCT CTGGCACTGT CATTGGCAGC | |
| | AATTGCTTTA TCACCAACAG TGTCATTGGC | |
| | CCCGGCTGCC ACATTGGTGA TAACGTGGTG | |
| | CTGGACCAGA CCTACCTGTG GCAGGGTGTT | |
| | CGAGTGGCGG CTGGAGCACA GATCCATCAG | |
| | TCTCTGCTTT GTGACAATGC TGAGGTCAAG | |
| | GAACGAGTGA CACTGAAACC ACGCTCTGTC | |
| | CTCACTTCCC AGGTGGTCGT GGGCCCAAAT | |
| | ATCACGCTGC CTGAGGGCTC GGTGATCTCT | |
| | TTGCACCCTC CAGATGCAGA GGAAGATGAA | |
| | GATGATGGCG AGTTCAGTGA TGATTCTGGG | |
| | GCTGACCAAG AAAAGGACAA AGTGAAGATG | |
| | AAAGGTTACA ATCCAGCAGA AGTAGGAGCT | |
| | GCTGGCAAGG GCTACCTCTG GAAAGCTGCA | |
| | GGCATGAACA TGGAGGAAGA GGAGGAACTG | |
| | CAGCAGAATC TGTGGGGACT CAAGATCAAC | |
| | ATGGAAGAAG AGAGTGAAAG TGAAAGTGAG | |
| | CAAAGTATGG ATTCTGAGGA GCCGGACAGC | |
| | CGGGGAGGCT CCCCTCAGAT GGATGACATC | |
| | AAAGTGTTCC AGAATGAAGT TTTAGGAACA | |
| | CTACAGCGGG GCAAAGAGGA GAACATTTCT | |
| | TGTGACAATC TCGTCCTGGA AATCAACTCT | |
| | CTCAAGTATG CCTATAACAT AAGTCTAAAG | |
| | GAGGTGATGC AGGTACTGAG CCACGTGGTC | |
| | CTGGAGTTCC CCCTGCAACA GATGGATTCC | |
| | CCGCTTGACT CAAGCCGCTA CTGTGCCCTG | |
| | CTGCTTCCTC TGCTAAAGGC CTGGAGCCCT | |
| | GTTTTTAGGA ACTACATAAA GCGCGCAGCC | |
| | GACCATTTGG AAGCGTTAGC AGCCATTGAG | |
| | GACTTCTTCC TAGAGCATGA AGCTCTTGGT | |
| | ATTTCCATGG CCAAGGTACT GATGGCTTTC | |
| | TACCAGCTGG AGATCCTGGC TGAGGAAACA | |
| | ATTCTGAGCT GGTTCAGCCA AAGAGATACA | |
| | ACTGACAAGG GCCAGCAGTT GCGCAAGAAT | |
| | CAACAGCTGC AGAGGTTCAT CCAGTGGCTA | |
| | AAAGAGGCAG AAGAGGAGTC ATCTGAAGAT | |
| | GACTGA | |
| elF2B5 | MAAPVVAPPGVVVSRANKRSGAGPGGSGGGGAR | 2 |
| protein | GAEEEPPPPLQAVLVADSFDRRFFPISKDQPRV | |
| | LLPLANVALIDYTLEFLTATGVQETFVFCCWKA | |
| | AQIKEHLLKSKWCRPTSLNVVRIITSELYRSLG | |
| | DVLRDVDAKALVRSDFLLVYGDVISNINITRAL | |
| | EEHRLRRKLEKNVSVMTMIFKESSPSHPTRCHE | |
| | DNVVVAVDSTTNRVLHFQKTQGLRRFAFPLSLF | |
| | QGSSDGVEVRYDLLDCHISICSPQVAQLFTDNF | |
| | DYQTRDDFVRGLLVNEEILGNQIHMHVTAKEYG | |
| | ARVSNLHMYSAVCADVIRRWVYPLTPEANFTDS | |
| | TTQSCTHSRHNIYRGPEVSLGHGSILEENVLLG | |
| | SGTVIGSNCFITNSVIGPGCHIGDNVVLDQTYL | |
| | WQGVRVAAGAQIHQSLLCDNAEVKERVTLKPRS | |
| | VLTSQVVVGPNITLPEGSVISLHPPDAEEDEDD | |
| | GEFSDDSGADQEKDKVKMKGYNPAEVGAAGKGY | |
| | LWKAAGMNMEEEEELQQNLWGLKINMEEESESE | |
| | SEQSMDSEEPDSRGGSPQMDDIKVFQNEVLGTL | |
| | QRGKEENISCDNLVLEINSLKYAYNISLKEVMQ | |
| | VLSHVVLEFPLQQMDSPLDSSRYCALLLPLLKA | |
| | WSPVFRNYIKRAADHLEALAAIEDFFLEHEALG | |
| | ISMAKVLMAFYQLEILAEETILSWFSQRDTTDK | |
| | GQQLRKNQQLQRFIQWLKEAEEESSEDD | |
| CAG | CGTT | 3 |
| promoter | ACATAACTTA CGGTAAATGG CCCGCCTGGC | |
| (comprises | TGACCGCCCA ACGACCCCCG CCCATTGACG | |
| a CMV | TCAATAATGA CGTATGTTCC CATAGTAACG | |
| enhancer | CCAATAGGGA CTTTCCATTG ACGTCAATGG | |
| (nts | GTGGACTATT TACGGTAAAC TGCCCACTTG | |
| 1-306 | GCAGTACATC AAGTGTATCA TATGCCAAGT | |
| of | ACGCCCCCTA TTGACGTCAA TGACGGTAAA | |
| SEQ ID | TGGCCCGCCT GGCATTATGC CCAGTACATG | |
| NO: 3) | ACCTTATGGG ACTTTCCTAC TTGGCAGTAC | |
| and a | ATCTACGTAT TAGTCATCGC TATTACCATG | |
| CBA | GGTCGAGGTG AGCCCCACGT TCTGCTTCAC | |
| promoter | TCTCCCCATC TCCCCCCCCT CCCCACCCCC | |
| (nt | AATTTTGTAT TTATTTATTT TTTAATTATT | |
| 307-581 | TTGTGCAGCG ATGGGGGGGG GGGGGGGGGG | |
| of SEQ | GGCGCGCGCC AGGCGGGGCG GGGCGGGGCG | |
| ID NO: | AGGGGCGGGG CGGGGCGAGG CGGAGAGGTG | |
| 3) | CGGCGGCAGC CAATCAGAGC GGCGCGCTCC | |
| | GAAAGTTTCC TTTTATGGCG AGGCGGCGGC | |
| | GGCGGCGGCC CTATAAAAAG CGAAGCGCGC | |
| | GGCGGGC | |
| gfaABC1D | GGTA | 4 |
| promoter | CCAACATATC CTGGTGTGGA GTAGGGGACG | |
| (647 nt) | CTGCTCTGAC AGAGGCTCGG GGGCCTGAGC | |
| | TGGCTCTGTG AGCTGGGGAG GAGGCAGACA | |
| | GCCAGGCCTT GTCTGCAAGC AGACCTGGCA | |
| | GCATTGGGCT GGCCGCCCCC CAGGGCCTCC | |
| | TCTTCATGCC CAGTGAATGA CTCACCTTGG | |
| | CACAGACACA ATGTTCGGGG TGGGCACAGT | |
| | GCCTGCTTCC CGCCGCACCC CAGCCCCCCT | |
| | CAAATGCCTT CCGAGAAGCC CATTGAGCAG | |
| | GGGGCTTGCA TTGCACCCCA GCCTGACAGC | |
| | CTGGCATCTT GGGATAAAAG CAGCACAGCC | |
| | CCCTAGGGGC TGCCCTTGCT GTGTGGCGCC | |
| | ACCGGCGGTG GAGAACAAGG CTCTATTCAG | |
| | CCTGTGCCCA GGAAAGGGGA TCAGGGGATG | |
| | CCCAGGCATG GACAGTGGGT GGCAGGGGGG | |
| | GAGAGGAGGG CTGTCTGCTT CCCAGAAGTC | |
| | CAAGGACACA AATGGGTGAG GGGAGAGCTC | |
| | TCCCCATAGC TGGGCTGCGG CCCAACCCCA | |
| | CCCCCTCAGG CTATGCCAGG GGGTGTTGCC | |
| | AGGGGCACCC GGGCATCGCC AGTCTAGCCC | |
| | ACTCCTTCAT AAAGCCCTCG CATCCCAGGA | |
| | GCGAGCAGAG CCA | |
| GFAP | CCCA | 5 |
| promoter | CCTCCCTCTC TGTGCTGGGA CTCACAGAGG | |
| (2208 nt) | GAGACCTCAG GAGGCAGTCT GTCCATCACA | |
| | TGTCCAAATG CAGAGCATAC CCTGGGCTGG | |
| | GCGCAGTGGC GCACAACTGT AATTCCAGCA | |
| | CTTTGGGAGG CTGATGTGGA AGGATCACTT | |
| | GAGCCCAGAA GTTCTAGACC AGCCTGGGCA | |
| | ACATGGCAAG ACCCTATCTC TACAAAAAAA | |
| | GTTAAAAAAT CAGCCACGTG TGGTGACACA | |
| | CACCTGTAGT CCCAGCTATT CAGGAGGCTG | |
| | AGGTGAGGGG ATCACTTAAG GCTGGGAGGT | |
| | TGAGGCTGCA GTGAGTCGTG GTTGCGCCAC | |
| | TGCACTCCAG CCTGGGCAAC AGTGAGACCC | |
| | TGTCTCAAAA GACAAAAAAA AAAAAAAAAA | |
| | AAAAAAGAAC ATATCCTGGT GTGGAGTAGG | |
| | GGACGCTGCT CTGACAGAGG CTCGGGGGCC | |
| | TGAGCTGGCT CTGTGAGCTG GGGAGGAGGC | |
| | AGACAGCCAG GCCTTGTCTG CAAGCAGACC | |
| | TGGCAGCATT GGGCTGGCCG CCCCCCAGGG | |
| | CCTCCTCTTC ATGCCCAGTG AATGACTCAC | |
| | CTTGGCACAG ACACAATGTT CGGGGTGGGC | |
| | ACAGTGCCTG CTTCCCGCCG CACCCCAGCC | |
| | CCCCTCAAAT GCCTTCCGAG AAGCCCATTG | |
| | AGCAGGGGGC TTGCATTGCA CCCCAGCCTG | |
| | ACAGCCTGGC ATCTTGGGAT AAAAGCAGCA | |
| | CAGCCCCCTA GGGGCTGCCC TTGCTGTGTG | |
| | GCGCCACCGG CGGTGGAGAA CAAGGCTCTA | |
| | TTCAGCCTGT GCCCAGGAAA GGGGATCAGG | |
| | GGATGCCCAG GCATGGACAG TGGGTGGCAG | |
| | GGGGGGAGAG GAGGGCTGTC TGCTTCCCAG | |
| | AAGTCCAAGG ACACAAATGG GTGAGGGGAC | |
| | TGGGCAGGGT TCTGACCCTG TGGGACCAGA | |
| | GTGGAGGGCG TAGATGGACC TGAAGTCTCC | |
| | AGGGACAACA GGGCCCAGGT CTCAGGCTCC | |
| | TAGTTGGGCC CAGTGGCTCC AGCGTTTCCA | |
| | AACCCATCCA TCCCCAGAGG TTCTTCCCAT | |
| | CTCTCCAGGC TGATGTGTGG GAACTCGAGG | |
| | AAATAAATCT CCAGTGGGAG ACGGAGGGGT | |
| | GGCCAGGGAA ACGGGGCGCT GCAGGAATAA | |
| | AGACGAGCCA GCACAGCCAG CTCATGTGTA | |
| | ACGGCTTTGT GGAGCTGTCA AGGCCTGGTC | |
| | TCTGGGAGAG AGGCACAGGG AGGCCAGACA | |
| | AGGAAGGGGT GACCTGGAGG GACAGATCCA | |
| | GGGGCTAAAG TCCTGATAAG GCAAGAGAGT | |
| | GCCGGCCCCC TCTTGCCCTA TCAGGACCTC | |
| | CACTGCCACA TAGAGGCCAT GATTGACCCT | |
| | TAGACAAAGG GCTGGTGTCC AATCCCAGCC | |
| | CCCAGCCCCA GAACTCCAGG GAATGAATGG | |
| | GCAGAGAGCA GGAATGTGGG ACATCTGTGT | |
| | TCAAGGGAAG GACTCCAGGA GTCTGCTGGG | |
| | AATGAGGCCT AGTAGGAAAT GAGGTGGCCC | |
| | TTGAGGGTAC AGAACAGGTT CATTCTTCGC | |
| | CAAATTCCCA GCACCTTGCA GGCACTTACA | |
| | GCTGAGTGAG ATAATGCCTG GGTTATGAAA | |
| | TCAAAAAGTT GGAAAGCAGG TCAGAGGTCA | |
| | TCTGGTACAG CCCTTCCTTC CCTTTTTTTT | |
| | TTTTTTTTTT TGTGAGACAA GGTCTCTCTC | |
| | TGTTGCCCAG GCTGGAGTGG CGCAAACACA | |
| | GCTCACTGCA GCCTCAACCT ACTGGGCTCA | |
| | AGCAATCCTC CAGCCTCAGC CTCCCAAAGT | |
| | GCTGGGATTA CAAGCATGAG CCACCCCACT | |
| | CAGCCCTTTC CTTCCTTTTT AATTGATGCA | |
| | TAATAATTGT AAGTATTCAT CATGGTCCAA | |
| | CCAACCCTTT CTTGACCCAC CTTCCTAGAG | |
| | AGAGGGTCCT CTTGCTTCAG CGGTCAGGGC | |
| | CCCAGACCCA TGGTCTGGCT CCAGGTACCA | |
| | CCTGCCTCAT GCAGGAGTTG GCGTGCCCAG | |
| | GAAGCTCTGC CTCTGGGCAC AGTGACCTCA | |
| | GTGGGGTGAG GGGAGCTCTC CCCATAGCTG | |
| | GGCTGCGGCC CAACCCCACC CCCTCAGGCT | |
| | ATGCCAGGGG GTGTTGCCAG GGGCACCCGG | |
| | GCATCGCCAG TCTAGCCCAC TCCTTCATAA | |
| | AGCCCTCGCA TOCCAGGAGC GAGCAGAGCC | |
| | AGAGCAGGTT GGAGAGGAGA CGCATCACCT | |
| | CCGCTGCTCG CCGG | |
| gfa1405 | ACTAGTAACATATCCTGGTGTGGAGTAGGGGAC | 15 |
| promoter | GCTGCTCTGACAGAGGCTCGGGGGCCTGAGCTG | |
| (1405 nt) | GCTCTGTGAGCTGGGGAGGAGGCAGACAGCCAG | |
| | GCCTTGTCTGCAAGCAGACCTGGCAGCATTGGG | |
| | CTGGCCGCCCCCCAGGGCCTCCTCTTCATGCCC | |
| | AGTGAATGACTCACCTTGGCACAGACACAATGT | |
| | TCGGGGTGGGCACAGTGCCTGCTTCCCGCCGCA | |
| | CCCCAGCCCCCCTCAAATGCCTTCCGAGAAGCC | |
| | CATTGAGCAGGGGGCTTGCATTGCACCCCAGCC | |
| | TGACAGCCTGGCATCTTGGGATAAAAGCAGCAC | |
| | AGCCCCCTAGGGGCTGCCCTTGCTGTGTGGCGC | |
| | CACCGGCGGTGGAGAACAAGGCTCTATTCAGCC | |
| | TGTGCCCAGGAAAGGGGATCAGGGGATGCCCAG | |
| | GCATGGACAGTGGGTGGCAGGGGGGGAGAGGAG | |
| | GGCTGTCTGCTTCCCAGAAGTCCAAGGACACAA | |
| | ATGGGTGAGGGGGAGGGGTGGCCAGGGAAACGG | |
| | GGCGCTGCAGGAATAAAGACGAGCCAGCACAGC | |
| | CAGCTCATGTGTAACGGCTTTGTGGAGCTGTCA | |
| | AGGCCTGGTCTCTGGGAGAGAGGCACAGGGAGG | |
| | CCAGACAAGGAAGGGGTGACCTGGAGGGACAGA | |
| | TCCAGGGGCTAAAGTCCTGATAAGGCAAGAGAG | |
| | TGCCGGCCCCCTCTTGCCCTATCAGGACCTCCA | |
| | CTGCCACATAGAGGCCATGATTGACCCTTAGAC | |
| | AGCTGAGTGAGATAATGCCTGGGTTATGAAATC | |
| | AAAAAGTTGGAAAGCAGGTCAGAGGTCATCTGG | |
| | TACAGCCCTTCCTTCCCTTTTTTTTTTTTTTTT | |
| | TTGTGAGACAAGGTCTCTCTCTGTTGCCCAGGC | |
| | TGGAGTGGCGCAAACACAGCTCACTGCAGCCTC | |
| | AACCTACTGGGCTCAAGCAATCCTCCAGCCTCA | |
| | GCCTCCCAAAGTGCTGGGATTACAAGCATGAGC | |
| | CACCCCACTCAGCCCTTTCCTTCCTTTTTAATT | |
| | GATGCATAATAATTGTAAGTATTCATCATGGTC | |
| | CAACCAACCCTTTCTTGACCCACCTTCCTAGAG | |
| | AGAGGGTCCTCTTGCTTCAGCGGTCAGGGCCCC | |
| | AGACCCATGGTCTGGCTCCAGGTACCACCTGCC | |
| | TCATGCAGGAGTTGGCGTGCCCAGGAAGCTCTG | |
| | CCTCTGGGCACAGTGACCTCAGTGGGGTGAGGG | |
| | GAGCTCTCCCCATAGCTGGGCTGCGGCCCAACC | |
| | CCACCCCCTCAGGCTATGCCAGGGGGTGTTGCC | |
| | AGGGGCACCCGGGCATCGCCAGTCTAGCCCACT | |
| | CCTTCATAAAGCCCTCGCATCCCAGGAGCGAGC | |
| | AGAGCCAGAGCAGGTTGGAGAGGAGACGCATCA | |
| | CCTCCGCTGCTCGTCCGGA | |

In some aspects, a nucleic acid of the disclosure comprises a promoter, an SV40 intron, the elF2B5 open reading frame, and a polyA tail (Table 2). The sequences in Table 2 are the nucleotide sequences of the elF2B5 transgene sequences without the 5′ and 3′ ITR sequences because the transgene sequences, in various aspects, are used in self-complementary and/or single-stranded AAV viral vectors.

In some aspects, a nucleic acid of the disclosure comprises the nucleotide sequence set forth in any one of SEQ ID NOs: 6-8 and 16 (Table 2). In various aspects, the nucleic acid is a variant of the nucleotide sequence set forth in any one of SEQ ID NOs: 6-8 and 16. In some aspects, the variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 6-8 and 16.

TABLE 2
Sequences of the elF2B5 transgene sequences without 5′ and 3′ ITR sequences.

| Sequence | | SEQ ID |
|---|---|---|
| Name | Sequence (5′-3′) | NO: |
| CAG.elF2B5 | cgttacataacttacggtaaatggcccgcctggctgaccgcccaa | 6 |
| (without 5′ | cgacccccgcccattgacgtcaataatgacgtatgttcccatagt | |
| and 3′ ITRs) | aacgccaatagggactttccattgacgtcaatgggtggactattt | |
| | acggtaaactgcccacttggcagtacatcaagtgtatcatatgcc | |
| | aagtacgccccctattgacgtcaatgacggtaaatggcccgcctg | |
| | gcattatgcccagtacatgaccttatgggactttcctacttggca | |
| | gtacatctacgtattagtcatcgctattaccatgggtcgaggtga | |
| | gccccacgttctgcttcactctccccatctcccccccctccccac | |
| | ccccaattttgtatttatttattttttaattattttgtgcagcga | |
| | tgggggcggggggggggggggcgcgcgccaggcggggcggggggg | |
| | gcgaggggcggggcggggcgaggcggagaggtgcggcggcagcca | |
| | atcagagcggcgcgctccgaaagtttccttttatggcgaggcggc | |
| | ggcggcggcggccctataaaaagcgaagcgcgcggcgggcgtccg | |
| | gaactggtaagtttagtctttttgtcttttatttcaggtcccgga | |
| | tccggtggtggtgcaaatcaaagaactgctcctcagtggatgttg | |
| | cctttacttctaggcctgtacggaagtgttacttctgctctaaaa | |
| | gctgcggaattgtacccgcggccaattctgcagtcgacggtaccg | |
| | cgggcccgggatccaccggttcgccaccatggcggcccctgtagt | |
| | ggcgccgcctggtgtggtggttagtcgggctaacaagcgcagcgg | |
| | cgcggggccgggaggcagcggtggcgggggagccagaggggcgga | |
| | ggaggaaccgccgccgcccctacaagcagttctggtggccgatag | |
| | cttcgatcgccgcttcttccccatctccaaggaccagcctcgggt | |
| | cctcttgcccctggccaatgtggcattaattgactacactctgga | |
| | attcctgactgccacaggtgtacaggaaacatttgtcttttgttg | |
| | ctggaaagctgctcaaatcaaagaacatttactgaagtcaaagtg | |
| | gtgccgccctacatctctcaatgtggttcgaataattacatcaga | |
| | gctctatcgatcactgggagatgtcctccgtgatgttgatgccaa | |
| | ggctttggtgcgctctgactttcttctggtgtatggggatgtcat | |
| | ctcaaacatcaatatcaccagagcccttgaggaacacaggttgag | |
| | acggaagctagaaaaaaatgtttctgtgatgacgatgatcttcaa | |
| | ggagtcatcccccagccacccaactcgttgccacgaagacaatgt | |
| | ggtagtggctgtggatagtaccacaaacagggttctccattttca | |
| | gaagacccagggtctccggcgttttgcatttcctctgagcctgtt | |
| | tcagggcagtagtgatggagtggaggttcgatatgatttactgga | |
| | ttgtcatatcagcatctgttctcctcaggtggcacaactctttac | |
| | agacaactttgactaccaaactcgagatgactttgtgcgaggtct | |
| | cttagtgaatgaggagatcctagggaaccagatccacatgcacgt | |
| | aacagctaaggaatatggtgcccgtgtctccaacctacacatgta | |
| | ctcagctgtctgtgctgacgtcatccgccgatgggtctaccctct | |
| | caccccagaggcgaacttcactgacagcaccacccagagctgcac | |
| | tcattcccggcacaacatctaccgagggcctgaggtcagcctggg | |
| | ccatggcagcatcctagaggaaaatgtgctcctgggctctggcac | |
| | tgtcattggcagcaattgctttatcaccaacagtgtcattggccc | |
| | cggctgccacattggtgataacgtggtgctggaccagacctacct | |
| | gtggcagggtgttcgagtggcggctggagcacagatccatcagtc | |
| | tctgctttgtgacaatgctgaggtcaaggaacgagtgacactgaa | |
| | accacgctctgtcctcacttcccaggtggtcgtgggcccaaatat | |
| | cacgctgcctgagggctcggtgatctctttgcaccctccagatgc | |
| | agaggaagatgaagatgatggcgagttcagtgatgattctggggc | |
| | tgaccaagaaaaggacaaagtgaagatgaaaggttacaatccagc | |
| | agaagtaggagctgctggcaagggctacctctggaaagctgcagg | |
| | catgaacatggaggaagaggaggaactgcagcagaatctgtgggg | |
| | actcaagatc | |
| gfaABC(1)D. | aacatggaagaagagagtgaaagtgaaagtgagcaaagtatggat | 7 |
| elF2B5 | tctgaggagccggacagccggggaggctcccctcagatggatgac | |
| (without 5′ | atcaaagtgttccagaatgaagttttaggaacactacagcggggc | |
| and 3′ ITRs) | aaagaggagaacatttcttgtgacaatctcgtcctggaaatcaac | |
| | tctctcaagtatgcctataacataagtctaaaggaggtgatgcag | |
| | gtactgagccacgtggtcctggagttccccctgcaacagatggat | |
| | tccccgcttgactcaagccgctactgtgccctgctgcttcctctg | |
| | ctaaaggcctggagccctgtttttaggaactacataaagcgcgca | |
| | gccgaccatttggaagcgttagcagccattgaggacttcttccta | |
| | gagcatgaagctcttggtatttccatggccaaggtactgatggct | |
| | ttctaccagctggagatcctggctgaggaaacaattctgagctgg | |
| | ttcagccaaagagatacaactgacaagggccagcagttgcgcaag | |
| | aatcaacagctgcagaggttcatccagtggctaaaagaggcagaa | |
| | gaggagtcatctgaagatgactgacgcgcgcgagatctacgggtg | |
| | gcatccctgtgacccctccccagtgcctctcctggccctggaagt | |
| | tgccactccagtgcccaccagccttgtcctaataaaattaagttg | |
| | catcattttgtctgactaggtgtccttctataatattatggggtg | |
| | gaggggggtggtatggagcaaggggcaagttgggaagacaacctg | |
| | tagggcctgcggggtctattgggaaccaagctggagtgcagtggc | |
| | acaatcttggctcactgcaatctccgcctcctgggttcaagcgat | |
| | tctcctgcctcagcctcccgagttgttgggattccaggcatgcat | |
| | gaccaggctcagctaatttttgtttttttggtagagacggggttt | |
| | caccatattggccaggctggtctccaactcctaatctcaggtgat | |
| | ctacccaccttggcctcccaaattgctgggattacaggcgtgaac | |
| | cactgctcccttccctgtccttctgattttgtaggtaaccacgtg | |
| | cggaccgaggtaccaacatatcctggtgtggagtaggggacgctg | |
| | ctctgacagaggctcgggggcctgagctggctctgtgagctgggg | |
| | aggaggcagacagccaggccttgtctgcaagcagacctggcagca | |
| | ttgggctggccgccccccagggcctcctcttcatgcccagtgaat | |
| | gactcaccttggcacagacacaatgttcggggtgggcacagtgcc | |
| | tgcttcccgccgcaccccagcccccctcaaatgccttccgagaag | |
| | cccattgagcagggggcttgcattgcaccccagcctgacagcctg | |
| | gcatcttgggataaaagcagcacagccccctaggggctgcccttg | |
| | ctgtgtggcgccaccggcggtggagaacaaggctctattcagcct | |
| | gtgcccaggaaaggggatcaggggatgcccaggcatggacagtgg | |
| | gtggcagggggggagaggagggctgtctgcttcccagaagtccaa | |
| | ggacacaaatgggtgaggggagagctctccccatagctgggctgc | |
| | ggcccaaccccaccccctcaggctatgccagggggtgttgccagg | |
| | ggcacccgggcatcgccagtctagcccactccttcataaagccct | |
| | cgcatcccaggagcgagcagagccatccggaactggtaagtttag | |
| | tctttttgtcttttatttcaggtcccggatccggtggtggtgcaa | |
| | atcaaagaactgctcctcagtggatgttgcctttacttctaggcc | |
| | tgtacggaagtgttacttctgctctaaaagctgcggaattgtacc | |
| | cgcggccaattctgcagtcgacggtaccgcgggcccgggatccac | |
| | cggttcgccaccatggcggcccctgtagtggcgccgcctggtgtg | |
| | gtggttagtcgggctaacaagcgcagcggcgcggggccgggaggc | |
| | agcggtggcgggggagccagaggggcggaggaggaaccgccgccg | |
| | cccctacaagcagttctggtggccgatagcttcgatcgccgcttc | |
| | ttccccatctccaaggaccagcctcgggtcctcttgcccctggcc | |
| | aatgtggcattaattgactacactctggaattcctgactgccaca | |
| | ggtgtacaggaaacatttgtcttttgttgctggaaagctgctcaa | |
| | atcaaagaacatttactgaagtcaaagtggtgccgccctacatct | |
| | ctcaatgtggttcgaataattacatcagagctctatcgatcactg | |
| | ggagatgtcctccgtgatgttgatgccaaggctttggtgcgctct | |
| | gactttcttctggtgtatggggatgtcatctcaaacatcaatatc | |
| | accagagcccttgaggaacacaggttgagacggaagctagaaaaa | |
| | aatgtttctgtgatgacgatgatcttcaaggagtcatcccccagc | |
| | cacccaactcgttgccacgaagacaatgtggtagtggctgtggat | |
| | agtaccacaaacagggttctccattttcagaagacccagggtctc | |
| | cggcgttttgcatttcctctgagcctgtttcagggcagtagtgat | |
| | ggagtggaggttcgatatgatttactggattgtcatatcagcatc | |
| | tgttctcctcaggtggcacaactctttacagacaactttgactac | |
| | caaactcgagatgactttgtgcgaggtctcttagtgaatgaggag | |
| | atcctagggaaccagatccacatgcacgtaacagctaaggaatat | |
| | ggtgcccgtgtctccaacctacacatgtactcagctgtctgtgct | |
| | gacgtcatccgccgatgggtctaccctctcaccccagaggcgaac | |
| | ttcactgacagcaccacccagagctgcactcattcccggcacaac | |
| | atctaccgagggcctgaggtcagcctgggccatggcagcatccta | |
| | gaggaaaatgtgctcctgggctctggcactgtcattggcagcaat | |
| | tgctttatcaccaacagtgtcattggccccggctgccacattggt | |
| | gataacgtggtgctggaccagacctacctgtggcagggtgttcga | |
| | gtggcggctggagcacagatccatcagtctctgctttgtgacaat | |
| | gctgaggtcaaggaacgagtgacactgaaaccacgctctgtcctc | |
| | acttcccaggtggtcgtgggcccaaatatcacgctgcctgagggc | |
| | tcggtgatctctttgcaccctccagatgcagaggaagatgaagat | |
| | gatggcgagttcagtgatgattctggggctgaccaagaaaaggac | |
| | aaagtgaagatgaaaggttacaatccagcagaagtaggagctgct | |
| | ggcaagggctacctctggaaagctgcaggcatgaacatggaggaa | |
| | gaggaggaactgcagcagaatctgtggggactcaagatcaacatg | |
| | gaagaagagagtgaaagtgaaagtgagcaaagtatggattctgag | |
| | gagccggacagccggggaggctcccctcagatggatgacatcaaa | |
| | gtgttccagaatgaagttttaggaacactacagcggggcaaagag | |
| | gagaacatttcttgtgacaatctcgtcctggaaatcaactctctc | |
| | aagtatgcctataacataagtctaaaggaggtgatgcaggtactg | |
| | agccacgtggtcctggagttccccctgcaacagatggattccccg | |
| | cttgactcaagccgctactgtgccctgctgcttcctctgctaaag | |
| | gcctggagccctgtttttaggaactacataaagcgcgcagccgac | |
| | catttggaagcgttagcagccattgaggacttcttcctagagcat | |
| | gaagctcttggtatttccatggccaaggtactgatggctttctac | |
| | cagctggagatcctggctgaggaaacaattctgagctggttcagc | |
| | caaagagatacaactgacaagggccagcagttgcgcaagaatcaa | |
| | cagctgcagaggttcatccagtggctaaaagaggcagaagaggag | |
| | tcatctgaagatgactgacgcgcgcgagatctacgggtggcatcc | |
| | ctgtgacccctccccagtgcctctcctggccctggaagttgccac | |
| | tccagtgcccaccagccttgtcctaataaaattaagttgcatcat | |
| | tttgtctgactaggtgtccttctataatattatggggtggagggg | |
| | ggtggtatggagcaaggggcaagttgggaagacaacctgtagggc | |
| | ctgcggggtctattgggaaccaagctggagtgcagtggcacaatc | |
| | ttggctcactgcaatctccgcctcctgggttcaagcgattctcct | |
| | gcctcagcctcccgagttgttgggattccaggcatgcatgaccag | |
| | gctcagctaatttttgtttttttggtagagacggggtttcaccat | |
| | attggccaggctggtctccaactcctaatctcaggtgatctaccc | |
| | accttggcctcccaaattgctgggattacaggcgtgaaccactgc | |
| | tcccttccctgtccttctgattttgtaggtaaccacgtgcggacc | |
| | ga | |
| GFAP. | cccacctccctctctgtgctgggactcacagagggagacctcagg | 8 |
| elF2B5 | aggcagtctgtccatcacatgtccaaatgcagagcataccctggg | |
| (without 5′ | ctgggcgcagtggcgcacaactgtaattccagcactttgggaggc | |
| and 3′ ITRs) | tgatgtggaaggatcacttgagcccagaagttctagaccagcctg | |
| | ggcaacatggcaagaccctatctctacaaaaaaagttaaaaaatc | |
| | agccacgtgtggtgacacacacctgtagtcccagctattcaggag | |
| | gctgaggtgaggggatcacttaaggctgggaggttgaggctgcag | |
| | tgagtcgtggttgcgccactgcactccagcctgggcaacagtgag | |
| | accctgtctcaaaagacaaaaaaaaaaaaaaaaaaaaaaagaaca | |
| | tatcctggtgtggagtaggggacgctgctctgacagaggctcggg | |
| | ggcctgagctggctctgtgagctggggaggaggcagacagccagg | |
| | ccttgtctgcaagcagacctggcagcattgggctggccgcccccc | |
| | agggcctcctcttcatgcccagtgaatgactcaccttggcacaga | |
| | cacaatgttcgggggggcacagtgcctgcttcccgccgcacccca | |
| | gcccccctcaaatgccttccgagaagcccattgagcagggggctt | |
| | gcattgcaccccagcctgacagcctggcatcttgggataaaagca | |
| | gcacagccccctaggggctgcccttgctgtgtggcgccaccggcg | |
| | gtggagaacaaggctctattcagcctgtgcccaggaaaggggatc | |
| | aggggatgcccaggcatggacagtgggtggcagggggggagagga | |
| | gggctgtctgcttcccagaagtccaaggacacaaatgggtgaggg | |
| | gactgggcagggttctgaccctgtgggaccagagtggagggcgta | |
| | gatggacctgaagtctccagggacaacagggcccaggtctcaggc | |
| | tcctagttgggcccagtggctccagcgtttccaaacccatccatc | |
| | cccagaggttcttcccatctctccaggctgatgtgtgggaactcg | |
| | aggaaataaatctccagtgggagacggaggggtggccagggaaac | |
| | ggggcgctgcaggaataaagacgagccagcacagccagctcatgt | |
| | gtaacggctttgtggagctgtcaaggcctggtctctgggagagag | |
| | gcacagggaggccagacaaggaaggggtgacctggagggacagat | |
| | ccaggggctaaagtcctgataaggcaagagagtgccggccccctc | |
| | ttgccctatcaggacctccactgccacatagaggccatgattgac | |
| | ccttagacaaagggctggtgtccaatcccagcccccagccccaga | |
| | actccagggaatgaatgggcagagagcaggaatgtgggacatctg | |
| | tgttcaagggaaggactccaggagtctgctgggaatgaggcctag | |
| | taggaaatgaggtggcccttgagggtacagaacaggttcattctt | |
| | cgccaaattcccagcaccttgcaggcacttacagctgagtgagat | |
| | aatgcctgggttatgaaatcaaaaagttggaaagcaggtcagagg | |
| | tcatctggtacagcccttccttccctttttttttttttttttttg | |
| | tgagacaaggtctctctctgttgcccaggctggagtggcgcaaac | |
| | acagctcactgcagcctcaacctactgggctcaagcaatcctcca | |
| | gcctcagcctcccaaagtgctgggattacaagcatgagccacccc | |
| | actcagccctttccttcctttttaattgatgcataataattgtaa | |
| | gtattcatcatggtccaaccaaccctttcttgacccaccttccta | |
| | gagagagggtcctcttgcttcagcggtcagggccccagacccatg | |
| | gtctggctccaggtaccacctgcctcatgcaggagttggcgtgcc | |
| | caggaagctctgcctctgggcacagtgacctcagtggggtgaggg | |
| | gagctctccccatagctgggctgcggcccaaccccaccccctcag | |
| | gctatgccagggggtgttgccaggggcacccgggcatcgccagtc | |
| | tagcccactccttcataaagccctcgcatcccaggagcgagcaga | |
| | gccagagcaggttggagaggagacgcatcacctccgctgctcgcc | |
| | ggtccggaactggtaagtttagtctttttgtcttttatttcaggt | |
| | cccggatccggtggtggtgcaaatcaaagaactgctcctcagtgg | |
| | atgttgcctttacttctaggcctgtacggaagtgttacttctgct | |
| | ctaaaagctgcggaattgtacccgcggccaattctgcagtcgacg | |
| | gtaccgcgggcccgggatccaccggttcgccaccatggcggcccc | |
| | tgtagtggcgccgcctggtgtggtggttagtcgggctaacaagcg | |
| | cagcggcgcggggccgggaggcagcggtggcgggggagccagagg | |
| | ggcggaggaggaaccgccgccgcccctacaagcagttctggtggc | |
| | cgatagcttcgatcgccgcttcttccccatctccaaggaccagcc | |
| | tcgggtcctcttgcccctggccaatgtggcattaattgactacac | |
| | tctggaattcctgactgccacaggtgtacaggaaacatttgtctt | |
| | ttgttgctggaaagctgctcaaatcaaagaacatttactgaagtc | |
| | aaagtggtgccgccctacatctctcaatgtggttcgaataattac | |
| | atcagagctctatcgatcactgggagatgtcctccgtgatgttga | |
| | tgccaaggctttggtgcgctctgactttcttctggtgtatgggga | |
| | tgtcatctcaaacatcaatatcaccagagcccttgaggaacacag | |
| | gttgagacggaagctagaaaaaaatgtttctgtgatgacgatgat | |
| | cttcaaggagtcatcccccagccacccaactcgttgccacgaaga | |
| | caatgtggtagtggctgtggatagtaccacaaacagggttctcca | |
| | ttttcagaagacccagggtctccggcgttttgcatttcctctgag | |
| | cctgtttcagggcagtagtgatggagtggaggttcgatatgattt | |
| | actggattgtcatatcagcatctgttctcctcaggtggcacaact | |
| | ctttacagacaactttgactaccaaactcgagatgactttgtgcg | |
| | aggtctcttagtgaatgaggagatcctagggaaccagatccacat | |
| | gcacgtaacagctaaggaatatggtgcccgtgtctccaacctaca | |
| | catgtactcagctgtctgtgctgacgtcatccgccgatgggtcta | |
| | ccctctcaccccagaggcgaacttcactgacagcaccacccagag | |
| | ctgcactcattcccggcacaacatctaccgagggcctgaggtcag | |
| | cctgggccatggcagcatcctagaggaaaatgtgctcctgggctc | |
| | tggcactgtcattggcagcaattgctttatcaccaacagtgtcat | |
| | tggccccggctgccacattggtgataacgtggtgctggaccagac | |
| | ctacctgtggcagggtgttcgagtggcggctggagcacagatcca | |
| | tcagtctctgctttgtgacaatgctgaggtcaaggaacgagtgac | |
| | actgaaaccacgctctgtcctcacttcccaggtggtcgtgggccc | |
| | aaatatcacgctgcctgagggctcggtgatctctttgcaccctcc | |
| | agatgcagaggaagatgaagatgatggcgagttcagtgatgattc | |
| | tggggctgaccaagaaaaggacaaagtgaagatgaaaggttacaa | |
| | tccagcagaagtagg | |
| gfa1405.elF | agctgctggcaagggctacctctggaaagctgcaggcatgaacat | 16 |
| 2B5 | ggaggaagaggaggaactgcagcagaatctgtggggactcaagat | |
| (without 5′ | caacatggaagaagagagtgaaagtgaaagtgagcaaagtatgga | |
| and 3′ ITRs) | ttctgaggagccggacagccggggaggctcccctcagatggatga | |
| | catcaaagtgttccagaatgaagttttaggaacactacagcgggg | |
| | caaagaggagaacatttcttgtgacaatctcgtcctggaaatcaa | |
| | ctctctcaagtatgcctataacataagtctaaaggaggtgatgca | |
| | ggtactgagccacgtggtcctggagttccccctgcaacagatgga | |
| | ttccccgcttgactcaagccgctactgtgccctgctgcttcctct | |
| | gctaaaggcctggagccctgtttttaggaactacataaagcgcgc | |
| | agccgaccatttggaagcgttagcagccattgaggacttcttcct | |
| | agagcatgaagctcttggtatttccatggccaaggtactgatggc | |
| | tttctaccagctggagatcctggctgaggaaacaattctgagctg | |
| | gttcagccaaagagatacaactgacaagggccagcagttgcgcaa | |
| | gaatcaacagctgcagaggttcatccagtggctaaaagaggcaga | |
| | agaggagtcatctgaagatgactgacgcgcgcgagatctacgggt | |
| | ggcatccctgtgacccctccccagtgcctctcctggccctggaag | |
| | ttgccactccagtgcccaccagccttgtcctaataaaattaagtt | |
| | gcatcattttgtctgactaggtgtccttctataatattatggggt | |
| | ggaggggggtggtatggagcaaggggcaagttgggaagacaacct | |
| | gtagggcctgcggggtctattgggaaccaagctggagtgcagtgg | |
| | cacaatcttggctcactgcaatctccgcctcctgggttcaagcga | |
| | ttctcctgcctcagcctcccgagttgttgggattccaggcatgca | |
| | tgaccaggctcagctaatttttgtttttttggtagagacggggtt | |
| | tcaccatattggccaggctggtctccaactcctaatctcaggtga | |
| | tctacccaccttggcctcccaaattgctgggattacaggcgtgaa | |
| | ccactgctcccttccctgtccttctgattttgtaggtaaccacgt | |
| | gcggaccgaGCGGCCGCATCGATTGAATTCGAGCTCGGTACCTCG | |
| | CGAATACATCTAGATATCGGATCCACTAGTAACATATCCTGGTGT | |
| | GGAGTAGGGGACGCTGCTCTGACAGAGGCTCGGGGGCCTGAGCTG | |
| | GCTCTGTGAGCTGGGGAGGAGGCAGACAGCCAGGCCTTGTCTGCA | |
| | AGCAGACCTGGCAGCATTGGGCTGGCCGCCCCCCAGGGCCTCCTC | |
| | TTCATGCCCAGTGAATGACTCACCTTGGCACAGACACAATGTTCG | |
| | GGGTGGGCACAGTGCCTGCTTCCCGCCGCACCCCAGCCCCCCTCA | |
| | AATGCCTTCCGAGAAGCCCATTGAGCAGGGGGCTTGCATTGCACC | |
| | CCAGCCTGACAGCCTGGCATCTTGGGATAAAAGCAGCACAGCCCC | |
| | CTAGGGGCTGCCCTTGCTGTGTGGCGCCACCGGCGGTGGAGAACA | |
| | AGGCTCTATTCAGCCTGTGCCCAGGAAAGGGGATCAGGGGATGCC | |
| | CAGGCATGGACAGTGGGTGGCAGGGGGGGAGAGGAGGGCTGTCTG | |
| | CTTCCCAGAAGTCCAAGGACACAAATGGGTGAGGGGGAGGGGTGG | |
| | CCAGGGAAACGGGGCGCTGCAGGAATAAAGACGAGCCAGCACAGC | |
| | CAGCTCATGTGTAACGGCTTTGTGGAGCTGTCAAGGCCTGGTCTC | |
| | TGGGAGAGAGGCACAGGGAGGCCAGACAAGGAAGGGGTGACCTGG | |
| | AGGGACAGATCCAGGGGCTAAAGTCCTGATAAGGCAAGAGAGTGC | |
| | CGGCCCCCTCTTGCCCTATCAGGACCTCCACTGCCACATAGAGGC | |
| | CATGATTGACCCTTAGACAGCTGAGTGAGATAATGCCTGGGTTAT | |
| | GAAATCAAAAAGTTGGAAAGCAGGTCAGAGGTCATCTGGTACAGC | |
| | CCTTCCTTCCCTTTTTTTTTTTTTTTTTTGTGAGACAAGGTCTCT | |
| | CTCTGTTGCCCAGGCTGGAGTGGCGCAAACACAGCTCACTGCAGC | |
| | CTCAACCTACTGGGCTCAAGCAATCCTCCAGCCTCAGCCTCCCAA | |
| | AGTGCTGGGATTACAAGCATGAGCCACCCCACTCAGCCCTTTCCT | |
| | TCCTTTTTAATTGATGCATAATAATTGTAAGTATTCATCATGGTC | |
| | CAACCAACCCTTTCTTGACCCACCTTCCTAGAGAGAGGGTCCTCT | |
| | TGCTTCAGCGGTCAGGGCCCCAGACCCATGGTCTGGCTCCAGGTA | |
| | CCACCTGCCTCATGCAGGAGTTGGCGTGCCCAGGAAGCTCTGCCT | |
| | CTGGGCACAGTGACCTCAGTGGGGTGAGGGGAGCTCTCCCCATAG | |
| | CTGGGCTGCGGCCCAACCCCACCCCCTCAGGCTATGCCAGGGGGT | |
| | GTTGCCAGGGGCACCCGGGCATCGCCAGTCTAGCCCACTCCTTCA | |
| | TAAAGCCCTCGCATCCCAGGAGCGAGCAGAGCCAGAGCAGGTTGG | |
| | AGAGGAGACGCATCACCTCCGCTGCTCGTCCGGAACTGGTAAGTT | |
| | TAGTCTTTTTGTCTTTTATTTCAGGTCCCGGATCCGGTGGTGGTG | |
| | CAAATCAAAGAACTGCTCCTCAGTGGATGTTGCCTTTACTTCTAG | |
| | GCCTGTACGGAAGTGTTACTTCTGCTCTAAAAGCTGCGGAATTGT | |
| | ACCCGCGGCCAATTCTGCAGTCGACGGTACCGCGGGCCCGGGATC | |
| | CACCGGTTCGCCACCATGGCGGCCCCTGTAGTGGCGCCGCCTGGT | |
| | GTGGTGGTTAGTCGGGCTAACAAGCGCAGCGGCGCGGGGCCGGGA | |
| | GGCAGCGGTGGCGGGGGAGCCAGAGGGGCGGAGGAGGAACCGCCG | |
| | CCGCCCCTACAAGCAGTTCTGGTGGCCGATAGCTTCGATCGCCGC | |
| | TTCTTCCCCATCTCCAAGGACCAGCCTCGGGTCCTCTTGCCCCTG | |
| | GCCAATGTGGCATTAATTGACTACACTCTGGAATTCCTGACTGCC | |
| | ACAGGTGTACAGGAAACATTTGTCTTTTGTTGCTGGAAAGCTGCT | |
| | CAAATCAAAGAACATTTACTGAAGTCAAAGTGGTGCCGCCCTACA | |
| | TCTCTCAATGTGGTTCGAATAATTACATCAGAGCTCTATCGATCA | |
| | CTGGGAGATGTCCTCCGTGATGTTGATGCCAAGGCTTTGGTGCGC | |
| | TCTGACTTTCTTCTGGTGTATGGGGATGTCATCTCAAACATCAAT | |
| | ATCACCAGAGCCCTTGAGGAACACAGGTTGAGACGGAAGCTAGAA | |
| | AAAAATGTTTCTGTGATGACGATGATCTTCAAGGAGTCATCCCCC | |
| | AGCCACCCAACTCGTTGCCACGAAGACAATGTGGTAGTGGCTGTG | |
| | GATAGTACCACAAACAGGGTTCTCCATTTTCAGAAGACCCAGGGT | |
| | CTCCGGCGTTTTGCATTTCCTCTGAGCCTGTTTCAGGGCAGTAGT | |
| | GATGGAGTGGAGGTTCGATATGATTTACTGGATTGTCATATCAGC | |
| | ATCTGTTCTCCTCAGGTGGCACAACTCTTTACAGACAACTTTGAC | |
| | TACCAAACTCGAGATGACTTTGTGCGAGGTCTCTTAGTGAATGAG | |
| | GAGATCCTAGGGAACCAGATCCACATGCACGTAACAGCTAAGGAA | |
| | TATGGTGCCCGTGTCTCCAACCTACACATGTACTCAGCTGTCTGT | |
| | GCTGACGTCATCCGCCGATGGGTCTACCCTCTCACCCCAGAGGCG | |
| | AACTTCACTGACAGCACCACCCAGAGCTGCACTCATTCCCGGCAC | |
| | AACATCTACCGAGGGCCTGAGGTCAGCCTGGGCCATGGCAGCATC | |
| | CTAGAGGAAAATGTGCTCCTGGGCTCTGGCACTGTCATTGGCAGC | |
| | AATTGCTTTATCACCAACAGTGTCATTGGCCCCGGCTGCCACATT | |
| | GGTGATAACGTGGTGCTGGACCAGACCTACCTGTGGCAGGGTGTT | |
| | CGAGTGGCGGCTGGAGCACAGATCCATCAGTCTCTGCTTTGTGAC | |
| | AATGCTGAGGTCAAGGAACGAGTGACACTGAAACCACGCTCTGTC | |
| | CTCACTTCCCAGGTGGTCGTGGGCCCAAATATCACGCTGCCTGAG | |
| | GGCTCGGTGATCTCTTTGCACCCTCCAGATGCAGAGGAAGATGAA | |
| | GATGATGGCGAGTTCAGTGATGATTCTGGGGCTGACCAAGAAAAG | |
| | GACAAAGTGAAGATGAAAGGTTACAATCCAGCAGAAGTAGGAGCT | |
| | GCTGGCAAGGGCTACCTCTGGAAAGCTGCAGGCATGAACATGGAG | |
| | GAAGAGGAGGAACTGCAGCAGAATCTGTGGGGACTCAAGATCAAC | |
| | ATGGAAGAAGAGAGTGAAAGTGAAAGTGAGCAAAGTATGGATTCT | |
| | GAGGAGCCGGACAGCCGGGGAGGCTCCCCTCAGATGGATGACATC | |
| | AAAGTGTTCCAGAATGAAGTTTTAGGAACACTACAGCGGGGCAAA | |
| | GAGGAGAACATTTCTTGTGACAATCTCGTCCTGGAAATCAACTCT | |
| | CTCAAGTATGCCTATAACATAAGTCTAAAGGAGGTGATGCAGGTA | |
| | CTGAGCCACGTGGTCCTGGAGTTCCCCCTGCAACAGATGGATTCC | |
| | CCGCTTGACTCAAGCCGCTACTGTGCCCTGCTGCTTCCTCTGCTA | |
| | AAGGCCTGGAGCCCTGTTTTTAGGAACTACATAAAGCGCGCAGCC | |
| | GACCATTTGGAAGCGTTAGCAGCCATTGAGGACTTCTTCCTAGAG | |
| | CATGAAGCTCTTGGTATTTCCATGGCCAAGGTACTGATGGCTTTC | |
| | TACCAGCTGGAGATCCTGGCTGAGGAAACAATTCTGAGCTGGTTC | |
| | AGCCAAAGAGATACAACTGACAAGGGCCAGCAGTTGCGCAAGAAT | |
| | CAACAGCTGCAGAGGTTCATCCAGTGGCTAAAAGAGGCAGAAGAG | |
| | GAGTCATCTGAAGATGACTGACGCGCGCGAGATCTACGGGTGGCA | |
| | TCCCTGTGACCCCTCCCCAGTGCCTCTCCTGGCCCTGGAAGTTGC | |
| | CACTCCAGTGCCCACCAGCCTTGTCCTAATAAAATTAAGTTGCAT | |
| | CATTTTGTCTGACTAGGTGTCCTTCTATAATATTATGGGGTGGAG | |
| | GGGGGTGGTATGGAGCAAGGGGCAAGTTGGGAAGACAACCTGTAG | |
| | GGCCTGCGGGGTCTATTGGGAACCAAGCTGGAGTGCAGTGGCACA | |
| | ATCTTGGCTCACTGCAATCTCCGCCTCCTGGGTTCAAGCGATTCT | |
| | CCTGCCTCAGCCTCCCGAGTTGTTGGGATTCCAGGCATGCATGAC | |
| | CAGGCTCAGCTAATTTTTGTTTTTTTGGTAGAGACGGGGTTTCAC | |
| | CATATTGGCCAGGCTGGTCTCCAACTCCTAATCTCAGGTGATCTA | |
| | CCCACCTTGGCCTCCCAAATTGCTGGGATTACAGGCGTGAACCAC | |
| | TGCTCCCTTCCCTGTCCTTCTGATTTTGTAGGTAACCACGTGCGG | |
| | ACCGAGCGGCCGC | |

In some aspects, a nucleic acid of the disclosure comprises a 5′ ITR, a promoter, an SV40 intron, the elF2B5 open reading frame, a polyA tail, and a 3′ ITR (Table 3). In some aspects, a nucleic acid of the disclosure comprises a nucleotide sequence comprising 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequences comprising a 5′ ITR, a promoter, an SV40 intron, the elF2B5 open reading frame, a polyA tail, and a 3′ ITR as set forth in Table 3.

In some aspects, a nucleic acid of the disclosure comprises the nucleotide sequence set forth in any one of SEQ ID NOs: 9-11 and 17 (Table 3). The sequences in Table 3 are the nucleotide sequences of the elF2B5 transgene sequences with the 5′ and 3′ ITR sequences. In various aspects, the nucleic acid is a variant of the nucleotide sequence set forth in any one of SEQ ID NOs: 9-11 and 17. In some aspects, the variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 9-11 and 17.

TABLE 3
Sequences of the elF2B5 transgene sequences with 5′ and 3′ ITR sequences.

| Sequence | | SEQ ID |
|---|---|---|
| Name | Sequence (5′-3′) | NO: |
| ITR- | cctgcaggcagctgcgcgctcgctcgctcactgaggccgcccggg | 9 |
| CAG.elF2B5 | cgtcgggcgacctttggtcgcccggcctcagtgagcgagcgagcg | |
| -ITR | cgcagagagggagtggccaactccatcactaggggttcctgcggc | |
| (with 5′ and | cgcatcgattgaattcgagctcggtacctcgcgaatacatctaga | |
| 3′ ITRs) | tatcggatccactagtcgttacataacttacggtaaatggcccgc | |
| | ctggctgaccgcccaacgacccccgcccattgacgtcaataatga | |
| | cgtatgttcccatagtaacgccaatagggactttccattgacgtc | |
| | aatgggtggactatttacggtaaactgcccacttggcagtacatc | |
| | aagtgtatcatatgccaagtacgccccctattgacgtcaatgacg | |
| | gtaaatggcccgcctggcattatgcccagtacatgaccttatggg | |
| | actttcctacttggcagtacatctacgtattagtcatcgctatta | |
| | ccatgggtcgaggtgagccccacgttctgcttcactctccccatc | |
| | tcccccccctccccacccccaattttgtatttatttattttttaa | |
| | ttattttgtgcagcgatgggggcggggggggggggggcgcgcgcc | |
| | aggcggggcggggcggggcgaggggcggggcggggcgaggcggag | |
| | aggtgcggcggcagccaatcagagcggcgcgctccgaaagtttcc | |
| | ttttatggcgaggcggcggcggcggcggccctataaaaagcgaag | |
| | cgcgcggcgggcgtccggaactggtaagtttagtctttttgtctt | |
| | ttatttcaggtcccggatccggtggtggtgcaaatcaaagaactg | |
| | ctcctcagtggatgttgcctttacttctaggcctgtacggaagtg | |
| | ttacttctgctctaaaagctgcggaattgtacccgcggccaattc | |
| | tgcagtcgacggtaccgcgggcccgggatccaccggttcgccacc | |
| | atggcggcccctgtagtggcgccgcctggtgtggtggttagtcgg | |
| | gctaacaagcgcagcggcgcggggccgggaggcagcggtggcggg | |
| | ggagccagaggggcggaggaggaaccgccgccgcccctacaagca | |
| | gttctggtggccgatagcttcgatcgccgcttcttccccatctcc | |
| | aaggaccagcctcgggtcctcttgcccctggccaatgtggcatta | |
| | attgactacactctggaattcctgactgccacaggtgtacaggaa | |
| | acatttgtcttttgttgctggaaagctgctcaaatcaaagaacat | |
| | ttactgaagtcaaagtggtgccgccctacatctctcaatgtggtt | |
| | cgaataattacatcagagctctatcgatcactgggagatgtcctc | |
| | cgtgatgttgatgccaaggctttggtgcgctctgactttcttctg | |
| | gtgtatggggatgtcatctcaaacatcaatatcaccagagccctt | |
| | gaggaacacaggttgagacggaagctagaaaaaaatgtttctgtg | |
| | atgacgatgatcttcaaggagtcatcccccagccacccaactcgt | |
| | tgccacgaagacaatgtggtagtggctgtggatagtaccacaaac | |
| | agggttctccattttcagaagacccagggtctccggcgttttgca | |
| | tttcctctgagcctgtttcagggcagtagtgatggagtggaggtt | |
| | cgatatgatttactggattgtcatatcagcatctgttctcctcag | |
| | gtggcacaactctttacagacaactttgactaccaaactcgagat | |
| | gactttgtgcgaggtctcttagtgaatgaggagatcctagggaac | |
| | cagatccacatgcacgtaacagctaaggaatatggtgcccgtgtc | |
| | tccaacctacacatgtactcagctgtctgtgctgacgtcatccg | |
| | ccgatgggtctaccctctcaccccagaggcgaacttcactgacag | |
| | caccacccagagctgcactcattcccggcacaacatctaccgagg | |
| | gcctgaggtcagcctgggccatggcagcatcctagaggaaaatgt | |
| | gctcctgggctctggcactgtcattggcagcaattgctttatcac | |
| | caacagtgtcattggccccggctgccacattggtgataacgtggt | |
| | gctggaccagacctacctgtggcagggtgttcgagtggcggctgg | |
| | agcacagatccatcagtctctgctttgtgacaatgctgaggtcaa | |
| | ggaacgagtgacactgaaaccacgctctgtcctcacttcccaggt | |
| | ggtcgtgggcccaaatatcacgctgcctgagggctcggtgatctc | |
| | tttgcaccctccagatgcagaggaagatgaagatgatggcgagtt | |
| | cagtgatgattctggggctgaccaagaaaaggacaaagtgaagat | |
| | gaaaggttacaatccagcagaagtaggagctgctggcaagggcta | |
| | cctctggaaagctgcaggcatgaacatggaggaagaggaggaact | |
| | gcagcagaatctgtggggactcaagatcaacatggaagaagagag | |
| | tgaaagtgaaagtgagcaaagtatggattctgaggagccggacag | |
| | ccggggaggctcccctcagatggatgacatcaaagtgttccagaa | |
| | tgaagttttaggaacactacagcggggcaaagaggagaacatttc | |
| | ttgtgacaatctcgtcctggaaatcaactctctcaagtatgccta | |
| | taacataagtctaaaggaggtgatgcaggtactgagccacgtggt | |
| | cctggagttccccctgcaacagatggattccccgcttgactcaag | |
| | ccgctactgtgccctgctgcttcctctgctaaaggcctggagccc | |
| | tgtttttaggaactacataaagcgcgcagccgaccatttggaagc | |
| | gttagcagccattgaggacttcttcctagagcatgaagctcttgg | |
| | tatttccatggccaaggtactgatggctttctaccagctggagat | |
| | cctggctgaggaaacaattctgagctggttcagccaaagagatac | |
| | aactgacaagggccagcagttgcgcaagaatcaacagctgcagag | |
| | gttcatccagtggctaaaagaggcagaagaggagtcatctgaaga | |
| | tgactgacgcgcgcgagatctacgggtggcatccctgtgacccct | |
| | ccccagtgcctctcctggccctggaagttgccactccagtgccca | |
| | ccagccttgtcctaataaaattaagttgcatcattttgtctgact | |
| | aggtgtccttctataatattatggggtggaggggggtggtatgga | |
| | gcaaggggcaagttgggaagacaacctgtagggcctgcggggtct | |
| | attgggaaccaagctggagtgcagtggcacaatcttggctcactg | |
| | caatctccgcctcctgggttcaagcgattctcctgcctcagcctc | |
| | ccgagttgttgggattccaggcatgcatgaccaggctcagctaat | |
| | ttttgtttttttggtagagacggggtttcaccatattggccaggc | |
| | tggtctccaactcctaatctcaggtgatctacccaccttggcctc | |
| | ccaaattgctgggattacaggcgtgaaccactgctcccttccctg | |
| | tccttctgattttgtaggtaaccacgtgcggaccgagcggccgca | |
| | ggaacccctagtgatggagttggccactccctctctgcgcgctcg | |
| | ctcgctcactgaggccgggcgaccaaaggtcgcccgacgcccggg | |
| | ctttgcccgggcggcctcagtgagcgagcgagcgcgcagctgcct | |
| | gcagg | |
| ITR- | cctgcaggcagctgcgcgctcgctcgctcactgaggccgcccggg | 10 |
| gfaABC(1)D. | cgtcgggcgacctttggtcgcccggcctcagtgagcgagcgagcg | |
| elF2B5-ITR | cgcagagagggagtggccaactccatcactaggggttcctgcggc | |
| (with 5′ and | cgcatcgattgaattcgagctcggtacctcgcgaatacatctaga | |
| 3′ ITRs) | tatcggatccactagtggtaccaacatatcctggtgtggagtagg | |
| | ggacgctgctctgacagaggctcgggggcctgagctggctctgtg | |
| | agctggggaggaggcagacagccaggccttgtctgcaagcagacc | |
| | tggcagcattgggctggccgccccccagggcctcctcttcatgcc | |
| | cagtgaatgactcaccttggcacagacacaatgttcggggtgggc | |
| | acagtgcctgcttcccgccgcaccccagcccccctcaaatgcctt | |
| | ccgagaagcccattgagcagggggcttgcattgcaccccagcctg | |
| | acagcctggcatcttgggataaaagcagcacagccccctaggggc | |
| | tgcccttgctgtgtggcgccaccggcggtggagaacaaggctcta | |
| | ttcagcctgtgcccaggaaaggggatcaggggatgcccaggcatg | |
| | gacagtgggtggcagggggggagaggagggctgtctgcttcccag | |
| | aagtccaaggacacaaatgggtgaggggagagctctccccatagc | |
| | tgggctgcggcccaaccccaccccctcaggctatgccagggggtg | |
| | ttgccaggggcacccgggcatcgccagtctagcccactccttcat | |
| | aaagccctcgcatcccaggagcgagcagagccatccggaactggt | |
| | aagtttagtctttttgtcttttatttcaggtcccggatccggtgg | |
| | tggtgcaaatcaaagaactgctcctcagtggatgttgcctttact | |
| | tctaggcctgtacggaagtgttacttctgctctaaaagctgcgga | |
| | attgtacccgcggccaattctgcagtcgacggtaccgcgggcccg | |
| | ggatccaccggttcgccaccatggcggcccctgtagtggcgccgc | |
| | ctggtgtggtggttagtcgggctaacaagcgcagcggcgcggggc | |
| | cgggaggcagcggtggcgggggagccagaggggcggaggaggaac | |
| | cgccgccgcccctacaagcagttctggtggccgatagcttcgatc | |
| | gccgcttcttccccatctccaaggaccagcctcgggtcctcttgc | |
| | ccctggccaatgtggcattaattgactacactctggaattcctga | |
| | ctgccacaggtgtacaggaaacatttgtcttttgttgctggaaag | |
| | ctgctcaaatcaaagaacatttactgaagtcaaagtggtgccgcc | |
| | ctacatctctcaatgtggttcgaataattacatcagagctctatc | |
| | gatcactgggagatgtcctccgtgatgttgatgccaaggctttgg | |
| | tgcgctctgactttcttctggtgtatggggatgtcatctcaaaca | |
| | tcaatatcaccagagcccttgaggaacacaggttgagacggaagc | |
| | tagaaaaaaatgtttctgtgatgacgatgatcttcaaggagtcat | |
| | cccccagccacccaactcgttgccacgaagacaatgtggtagtgg | |
| | ctgtggatagtaccacaaacagggttctccattttcagaagaccc | |
| | agggtctccggcgttttgcatttcctctgagcctgtttcagggca | |
| | gtagtgatggagtggaggttcgatatgatttactggattgtcata | |
| | tcagcatctgttctcctcaggtggcacaactctttacagacaact | |
| | ttgactaccaaactcgagatgactttgtgcgaggtctcttagtga | |
| | atgaggagatcctagggaaccagatccacatgcacgtaacagcta | |
| | aggaatatggtgcccgtgtctccaacctacacatgtactcagctg | |
| | tctgtgctgacgtcatccgccgatgggtctaccctctcaccccag | |
| | aggcgaacttcactgacagcaccacccagagctgcactcattccc | |
| | ggcacaacatctaccgagggcctgaggtcagcctgggccatggca | |
| | gcatcctagaggaaaatgtgctcctgggctctggcactgtcattg | |
| | gcagcaattgctttatcaccaacagtgtcattggccccggctgcc | |
| | acattggtgataacgtggtgctggaccagacctacctgtggcagg | |
| | gtgttcgagtggcggctggagcacagatccatcagtctctgcttt | |
| | gtgacaatgctgaggtcaaggaacgagtgacactgaaaccacgct | |
| | ctgtcctcacttcccaggtggtcgtgggcccaaatatcacgctgc | |
| | ctgagggctcggtgatctctttgcaccctccagatgcagaggaag | |
| | atgaagatgatggcgagttcagtgatgattctggggctgaccaag | |
| | aaaaggacaaagtgaagatgaaaggttacaatccagcagaagtag | |
| | gagctgctggcaagggctacctctggaaagctgcaggcatgaaca | |
| | tggaggaagaggaggaactgcagcagaatctgtggggactcaaga | |
| | tcaacatggaagaagagagtgaaagtgaaagtgagcaaagtatgg | |
| | attctgaggagccggacagccggggaggctcccctcagatggatg | |
| | acatcaaagtgttccagaatgaagttttaggaacactacagcggg | |
| | gcaaagaggagaacatttcttgtgacaatctcgtcctggaaatca | |
| | actctctcaagtatgcctataacataagtctaaaggaggtgatgc | |
| | aggtactgagccacgtggtcctggagttccccctgcaacagatgg | |
| | attccccgcttgactcaagccgctactgtgccctgctgcttcctc | |
| | tgctaaaggcctggagccctgtttttaggaactacataaagcgcg | |
| | cagccgaccatttggaagcgttagcagccattgaggacttcttcc | |
| | tagagcatgaagctcttggtatttccatggccaaggtactgatgg | |
| | ctttctaccagctggagatcctggctgaggaaacaattctgagct | |
| | ggttcagccaaagagatacaactgacaagggccagcagttgcgca | |
| | agaatcaacagctgcagaggttcatccagtggctaaaagaggcag | |
| | aagaggagtcatctgaagatgactgacgcgcgcgagatctacggg | |
| | tggcatccctgtgacccctccccagtgcctctcctggccctggaa | |
| | gttgccactccagtgcccaccagccttgtcctaataaaattaagt | |
| | tgcatcattttgtctgactaggtgtccttctataatattatgggg | |
| | tggaggggggtggtatggagcaaggggcaagttgggaagacaacc | |
| | tgtagggcctgcggggtctattgggaaccaagctggagtgcagtg | |
| | gcacaatcttggctcactgcaatctccgcctcctgggttcaagcg | |
| | attctcctgcctcagcctcccgagttgttgggattccaggcatgc | |
| | atgaccaggctcagctaatttttgtttttttggtagagacggggt | |
| | ttcaccatattggccaggctggtctccaactcctaatctcaggtg | |
| | atctacccaccttggcctcccaaattgctgggattacaggcgtga | |
| | accactgctcccttccctgtccttctgattttgtaggtaaccacg | |
| | tgcggaccgagcggccgcaggaacccctagtgatggagttggcca | |
| | ctccctctctgcgcgctcgctcgctcactgaggccgggcgaccaa | |
| | aggtcgcccgacgcccgggctttgcccgggcggcctcagtgagcg | |
| | agcgagcgcgcagctgcctgcagg | |
| ITR- | cctgcaggcagctgcgcgctcgctcgctcactgaggccgcccggg | 11 |
| GFAP.elF2B | cgtcgggcgacctttggtcgcccggcctcagtgagcgagcgagcg | |
| 5-ITR | cgcagagagggagtggccaactccatcactaggggttcctgcggc | |
| (with 5′ and | cgcatcgattgaattcgagctcggtacctcgcgaatacatctaga | |
| 3′ ITRs) | tatcggatccactagtcccacctccctctctgtgctgggactcac | |
| | agagggagacctcaggaggcagtctgtccatcacatgtccaaatg | |
| | cagagcataccctgggctgggcgcagtggcgcacaactgtaattc | |
| | cagcactttgggaggctgatgtggaaggatcacttgagcccagaa | |
| | gttctagaccagcctgggcaacatggcaagaccctatctctacaa | |
| | aaaaagttaaaaaatcagccacgtgtggtgacacacacctgtagt | |
| | cccagctattcaggaggctgaggtgaggggatcacttaaggctgg | |
| | gaggttgaggctgcagtgagtcgtggttgcgccactgcactccag | |
| | cctgggcaacagtgagaccctgtctcaaaagacaaaaaaaaaaaa | |
| | aaaaaaaaaaagaacatatcctggtgtggagtaggggacgctgct | |
| | ctgacagaggctcgggggcctgagctggctctgtgagctggggag | |
| | gaggcagacagccaggccttgtctgcaagcagacctggcagcatt | |
| | gggctggccgccccccagggcctcctcttcatgcccagtgaatga | |
| | ctcaccttggcacagacacaatgttcgggggggcacagtgcctgc | |
| | ttcccgccgcaccccagcccccctcaaatgccttccgagaagccc | |
| | attgagcagggggcttgcattgcaccccagcctgacagcctggca | |
| | tcttgggataaaagcagcacagccccctaggggctgcccttgctg | |
| | tgtggcgccaccggcggtggagaacaaggctctattcagcctgtg | |
| | cccaggaaaggggatcaggggatgcccaggcatggacagtgggtg | |
| | gcagggggggagaggagggctgtctgcttcccagaagtccaagga | |
| | cacaaatgggtgaggggactgggcagggttctgaccctgtgggac | |
| | cagagtggagggcgtagatggacctgaagtctccagggacaacag | |
| | ggcccaggtctcaggctcctagttgggcccagtggctccagcgtt | |
| | tccaaacccatccatccccagaggttcttcccatctctccaggct | |
| | gatgtgtgggaactcgaggaaataaatctccagtgggagacggag | |
| | gggtggccagggaaacggggcgctgcaggaataaagacgagccag | |
| | cacagccagctcatgtgtaacggctttgtggagctgtcaaggcct | |
| | ggtctctgggagagaggcacagggaggccagacaaggaaggggtg | |
| | acctggagggacagatccaggggctaaagtcctgataaggcaaga | |
| | gagtgccggccccctcttgccctatcaggacctccactgccacat | |
| | agaggccatgattgacccttagacaaagggctggtgtccaatccc | |
| | agcccccagccccagaactccagggaatgaatgggcagagagcag | |
| | gaatgtgggacatctgtgttcaagggaaggactccaggagtctgc | |
| | tgggaatgaggcctagtaggaaatgaggtggcccttgagggtaca | |
| | gaacaggttcattcttcgccaaattcccagcaccttgcaggcact | |
| | tacagctgagtgagataatgcctgggttatgaaatcaaaaagttg | |
| | gaaagcaggtcagaggtcatctggtacagcccttccttccctttt | |
| | tttttttttttttttgtgagacaaggtctctctctgttgcccagg | |
| | ctggagtggcgcaaacacagctcactgcagcctcaacctactggg | |
| | ctcaagcaatcctccagcctcagcctcccaaagtgctgggattac | |
| | aagcatgagccaccccactcagccctttccttcctttttaattga | |
| | tgcataataattgtaagtattcatcatggtccaaccaaccctttc | |
| | ttgacccaccttcctagagagagggtcctcttgcttcagcggtca | |
| | gggccccagacccatggtctggctccaggtaccacctgcctcatg | |
| | caggagttggcgtgcccaggaagctctgcctctgggcacagtgac | |
| | ctcagtggggtgaggggagctctccccatagctgggctgcggccc | |
| | aaccccaccccctcaggctatgccagggggtgttgccaggggcac | |
| | ccgggcatcgccagtctagcccactccttcataaagccctcgcat | |
| | cccaggagcgagcagagccagagcaggttggagaggagacgcatc | |
| | acctccgctgctcgccggtccggaactggtaagtttagtcttttt | |
| | gtcttttatttcaggtcccggatccggtggtggtgcaaatcaaag | |
| | aactgctcctcagtggatgttgcctttacttctaggcctgtacgg | |
| | aagtgttacttctgctctaaaagctgcggaattgtacccgcggcc | |
| | aattctgcagtcgacggtaccgcgggcccgggatccaccggttcg | |
| | ccaccatggcggcccctgtagtggcgccgcctggtgtggtggtta | |
| | gtcgggctaacaagcgcagcggcgcggggccgggaggcagcggtg | |
| | gcgggggagccagaggggcggaggaggaaccgccgccgcccctac | |
| | aagcagttctggtggccgatagcttcgatcgccgcttcttcccca | |
| | tctccaaggaccagcctcgggtcctcttgcccctggccaatgtgg | |
| | cattaattgactacactctggaattcctgactgccacaggtgtac | |
| | aggaaacatttgtcttttgttgctggaaagctgctcaaatcaaag | |
| | aacatttactgaagtcaaagtggtgccgccctacatctctcaatg | |
| | tggttcgaataattacatcagagctctatcgatcactgggagatg | |
| | tcctccgtgatgttgatgccaaggctttggtgcgctctgactttc | |
| | ttctggtgtatggggatgtcatctcaaacatcaatatcaccagag | |
| | cccttgaggaacacaggttgagacggaagctagaaaaaaatgttt | |
| | ctgtgatgacgatgatcttcaaggagtcatcccccagccacccaa | |
| | ctcgttgccacgaagacaatgtggtagtggctgtggatagtacca | |
| | caaacagggttctccattttcagaagacccagggtctccggcgtt | |
| | ttgcatttcctctgagcctgtttcagggcagtagtgatggagtgg | |
| | aggttcgatatgatttactggattgtcatatcagcatctgttctc | |
| | ctcaggtggcacaactctttacagacaactttgactaccaaactc | |
| | gagatgactttgtgcgaggtctcttagtgaatgaggagatcctag | |
| | ggaaccagatccacatgcacgtaacagctaaggaatatggtgccc | |
| | gtgtctccaacctacacatgtactcagctgtctgtgctgacgtca | |
| | tccgccgatgggtctaccctctcaccccagaggcgaacttcactg | |
| | acagcaccacccagagctgcactcattcccggcacaacatctacc | |
| | gagggcctgaggtcagcctgggccatggcagcatcctagaggaaa | |
| | atgtgctcctgggctctggcactgtcattggcagcaattgcttta | |
| | tcaccaacagtgtcattggccccggctgccacattggtgataacg | |
| | tggtgctggaccagacctacctgtggcagggtgttcgagtggcgg | |
| | ctggagcacagatccatcagtctctgctttgtgacaatgctgagg | |
| | tcaaggaacgagtgacactgaaaccacgctctgtcctcacttccc | |
| | aggtggtcgtgggcccaaatatcacgctgcctgagggctcggtga | |
| | tctctttgcaccctccagatgcagaggaagatgaagatgatggcg | |
| | agttcagtgatgattctggggctgaccaagaaaaggacaaagtga | |
| | agatgaaaggttacaatccagcagaagtaggagctgctggcaagg | |
| | gctacctctggaaagctgcaggcatgaacatggaggaagaggagg | |
| | aactgcagcagaatctgtggggactcaagatcaacatggaagaag | |
| | agagtgaaagtgaaagtgagcaaagtatggattctgaggagccgg | |
| | acagccggggaggctcccctcagatggatgacatcaaagtgttcc | |
| | agaatgaagttttaggaacactacagcggggcaaagaggagaaca | |
| | tttcttgtgacaatctcgtcctggaaatcaactctctcaagtatg | |
| | cctataacataagtctaaaggaggtgatgcaggtactgagccacg | |
| | tggtcctggagttccccctgcaacagatggattccccgcttgact | |
| | caagccgctactgtgccctgctgcttcctctgctaaaggcctgga | |
| | gccctgtttttaggaactacataaagcgcgcagccgaccatttgg | |
| | aagcgttagcagccattgaggacttcttcctagagcatgaagctc | |
| | ttggtatttccatggccaaggtactgatggctttctaccagctgg | |
| | agatcctggctgaggaaacaattctgagctggttcagccaaagag | |
| | atacaactgacaagggccagcagttgcgcaagaatcaacagctgc | |
| | agaggttcatccagtggctaaaagaggcagaagaggagtcatctg | |
| | aagatgactgacgcgcgcgagatctacgggtggcatccctgtgac | |
| | ccctccccagtgcctctcctggccctggaagttgccactccagtg | |
| | cccaccagccttgtcctaataaaattaagttgcatcattttgtct | |
| | gactaggtgtccttctataatattatggggtggaggggggtggta | |
| | tggagcaaggggcaagttgggaagacaacctgtagggcctgcggg | |
| | gtctattgggaaccaagctggagtgcagtggcacaatcttggctc | |
| | actgcaatctccgcctcctgggttcaagcgattctcctgcctcag | |
| | cctcccgagttgttgggattccaggcatgcatgaccaggctcagc | |
| | taatttttgtttttttggtagagacggggtttcaccatattggcc | |
| | aggctggtctccaactcctaatctcaggtgatctacccaccttgg | |
| | cctcccaaattgctgggattacaggcgtgaaccactgctcccttc | |
| | cctgtccttctgattttgtaggtaaccacgtgcggaccgagcggc | |
| | cgcaggaacccctagtgatggagttggccactccctctctgcgcg | |
| | ctcgctcgctcactgaggccgggcgaccaaaggtcgcccgacgcc | |
| | cgggctttgcccgggcggcctcagtgagcgagcgagcgcgcagct | |
| | gcctgcagg | |
| ITR- | CCTGCAGGCAGCTGCGCGCTCGCTCGCTCACTGAGGCCGCCCGGG | 17 |
| gfa1405.elF | CGTCGGGCGACCTTTGGTCGCCCGGCCTCAGTGAGCGAGCGAGCG | |
| 2B5-ITR | CGCAGAGAGGGAGTGGCCAACTCCATCACTAGGGGTTCCTGCGGC | |
| (with 5′ and | CGCATCGATTGAATTCGAGCTCGGTACCTCGCGAATACATCTAGA | |
| 3′ ITRs) | TATCGGATCCACTAGTAACATATCCTGGTGTGGAGTAGGGGACGC | |
| | TGCTCTGACAGAGGCTCGGGGGCCTGAGCTGGCTCTGTGAGCTGG | |
| | GGAGGAGGCAGACAGCCAGGCCTTGTCTGCAAGCAGACCTGGCAG | |
| | CATTGGGCTGGCCGCCCCCCAGGGCCTCCTCTTCATGCCCAGTGA | |
| | ATGACTCACCTTGGCACAGACACAATGTTCGGGGTGGGCACAGTG | |
| | CCTGCTTCCCGCCGCACCCCAGCCCCCCTCAAATGCCTTCCGAGA | |
| | AGCCCATTGAGCAGGGGGCTTGCATTGCACCCCAGCCTGACAGCC | |
| | TGGCATCTTGGGATAAAAGCAGCACAGCCCCCTAGGGGCTGCCCT | |
| | TGCTGTGTGGCGCCACCGGCGGTGGAGAACAAGGCTCTATTCAGC | |
| | CTGTGCCCAGGAAAGGGGATCAGGGGATGCCCAGGCATGGACAGT | |
| | GGGTGGCAGGGGGGGAGAGGAGGGCTGTCTGCTTCCCAGAAGTCC | |
| | AAGGACACAAATGGGTGAGGGGGAGGGGTGGCCAGGGAAACGGGG | |
| | CGCTGCAGGAATAAAGACGAGCCAGCACAGCCAGCTCATGTGTAA | |
| | CGGCTTTGTGGAGCTGTCAAGGCCTGGTCTCTGGGAGAGAGGCAC | |
| | AGGGAGGCCAGACAAGGAAGGGGTGACCTGGAGGGACAGATCCAG | |
| | GGGCTAAAGTCCTGATAAGGCAAGAGAGTGCCGGCCCCCTCTTGC | |
| | CCTATCAGGACCTCCACTGCCACATAGAGGCCATGATTGACCCTT | |
| | AGACAGCTGAGTGAGATAATGCCTGGGTTATGAAATCAAAAAGTT | |
| | GGAAAGCAGGTCAGAGGTCATCTGGTACAGCCCTTCCTTCCCTTT | |
| | TTTTTTTTTTTTTTTGTGAGACAAGGTCTCTCTCTGTTGCCCAGG | |
| | CTGGAGTGGCGCAAACACAGCTCACTGCAGCCTCAACCTACTGGG | |
| | CTCAAGCAATCCTCCAGCCTCAGCCTCCCAAAGTGCTGGGATTAC | |
| | AAGCATGAGCCACCCCACTCAGCCCTTTCCTTCCTTTTTAATTGA | |
| | TGCATAATAATTGTAAGTATTCATCATGGTCCAACCAACCCTTTC | |
| | TTGACCCACCTTCCTAGAGAGAGGGTCCTCTTGCTTCAGCGGTCA | |
| | GGGCCCCAGACCCATGGTCTGGCTCCAGGTACCACCTGCCTCATG | |
| | CAGGAGTTGGCGTGCCCAGGAAGCTCTGCCTCTGGGCACAGTGAC | |
| | CTCAGTGGGGTGAGGGGAGCTCTCCCCATAGCTGGGCTGCGGCCC | |
| | AACCCCACCCCCTCAGGCTATGCCAGGGGGTGTTGCCAGGGGCAC | |
| | CCGGGCATCGCCAGTCTAGCCCACTCCTTCATAAAGCCCTCGCAT | |
| | CCCAGGAGCGAGCAGAGCCAGAGCAGGTTGGAGAGGAGACGCATC | |
| | ACCTCCGCTGCTCGTCCGGAACTGGTAAGTTTAGTCTTTTTGTCT | |
| | TTTATTTCAGGTCCCGGATCCGGTGGTGGTGCAAATCAAAGAACT | |
| | GCTCCTCAGTGGATGTTGCCTTTACTTCTAGGCCTGTACGGAAGT | |
| | GTTACTTCTGCTCTAAAAGCTGCGGAATTGTACCCGCGGCCAATT | |
| | CTGCAGTCGACGGTACCGCGGGCCCGGGATCCACCGGTTCGCCAC | |
| | CATGGCGGCCCCTGTAGTGGCGCCGCCTGGTGTGGTGGTTAGTCG | |
| | GGCTAACAAGCGCAGCGGCGCGGGGCCGGGAGGCAGCGGTGGCGG | |
| | GGGAGCCAGAGGGGCGGAGGAGGAACCGCCGCCGCCCCTACAAGC | |
| | AGTTCTGGTGGCCGATAGCTTCGATCGCCGCTTCTTCCCCATCTC | |
| | CAAGGACCAGCCTCGGGTCCTCTTGCCCCTGGCCAATGTGGCATT | |
| | AATTGACTACACTCTGGAATTCCTGACTGCCACAGGTGTACAGGA | |
| | AACATTTGTCTTTTGTTGCTGGAAAGCTGCTCAAATCAAAGAACA | |
| | TTTACTGAAGTCAAAGTGGTGCCGCCCTACATCTCTCAATGTGGT | |
| | TCGAATAATTACATCAGAGCTCTATCGATCACTGGGAGATGTCCT | |
| | CCGTGATGTTGATGCCAAGGCTTTGGTGCGCTCTGACTTTCTTCT | |
| | GGTGTATGGGGATGTCATCTCAAACATCAATATCACCAGAGCCCT | |
| | TGAGGAACACAGGTTGAGACGGAAGCTAGAAAAAAATGTTTCTGT | |
| | GATGACGATGATCTTCAAGGAGTCATCCCCCAGCCACCCAACTCG | |
| | TTGCCACGAAGACAATGTGGTAGTGGCTGTGGATAGTACCACAAA | |
| | CAGGGTTCTCCATTTTCAGAAGACCCAGGGTCTCCGGCGTTTTGC | |
| | ATTTCCTCTGAGCCTGTTTCAGGGCAGTAGTGATGGAGTGGAGGT | |
| | TCGATATGATTTACTGGATTGTCATATCAGCATCTGTTCTCCTCA | |
| | GGTGGCACAACTCTTTACAGACAACTTTGACTACCAAACTCGAGA | |
| | TGACTTTGTGCGAGGTCTCTTAGTGAATGAGGAGATCCTAGGGAA | |
| | CCAGATCCACATGCACGTAACAGCTAAGGAATATGGTGCCCGTGT | |
| | CTCCAACCTACACATGTACTCAGCTGTCTGTGCTGACGTCATCCG | |
| | CCGATGGGTCTACCCTCTCACCCCAGAGGCGAACTTCACTGACAG | |
| | CACCACCCAGAGCTGCACTCATTCCCGGCACAACATCTACCGAGG | |
| | GCCTGAGGTCAGCCTGGGCCATGGCAGCATCCTAGAGGAAAATGT | |
| | GCTCCTGGGCTCTGGCACTGTCATTGGCAGCAATTGCTTTATCAC | |
| | CAACAGTGTCATTGGCCCCGGCTGCCACATTGGTGATAACGTGGT | |
| | GCTGGACCAGACCTACCTGTGGCAGGGTGTTCGAGTGGCGGCTGG | |
| | AGCACAGATCCATCAGTCTCTGCTTTGTGACAATGCTGAGGTCAA | |
| | GGAACGAGTGACACTGAAACCACGCTCTGTCCTCACTTCCCAGGT | |
| | GGTCGTGGGCCCAAATATCACGCTGCCTGAGGGCTCGGTGATCTC | |
| | TTTGCACCCTCCAGATGCAGAGGAAGATGAAGATGATGGCGAGTT | |
| | CAGTGATGATTCTGGGGCTGACCAAGAAAAGGACAAAGTGAAGAT | |
| | GAAAGGTTACAATCCAGCAGAAGTAGGAGCTGCTGGCAAGGGCTA | |
| | CCTCTGGAAAGCTGCAGGCATGAACATGGAGGAAGAGGAGGAACT | |
| | GCAGCAGAATCTGTGGGGACTCAAGATCAACATGGAAGAAGAGAG | |
| | TGAAAGTGAAAGTGAGCAAAGTATGGATTCTGAGGAGCCGGACAG | |
| | CCGGGGAGGCTCCCCTCAGATGGATGACATCAAAGTGTTCCAGAA | |
| | TGAAGTTTTAGGAACACTACAGCGGGGCAAAGAGGAGAACATTTC | |
| | TTGTGACAATCTCGTCCTGGAAATCAACTCTCTCAAGTATGCCTA | |
| | TAACATAAGTCTAAAGGAGGTGATGCAGGTACTGAGCCACGTGGT | |
| | CCTGGAGTTCCCCCTGCAACAGATGGATTCCCCGCTTGACTCAAG | |
| | CCGCTACTGTGCCCTGCTGCTTCCTCTGCTAAAGGCCTGGAGCCC | |
| | TGTTTTTAGGAACTACATAAAGCGCGCAGCCGACCATTTGGAAGC | |
| | GTTAGCAGCCATTGAGGACTTCTTCCTAGAGCATGAAGCTCTTGG | |
| | TATTTCCATGGCCAAGGTACTGATGGCTTTCTACCAGCTGGAGAT | |
| | CCTGGCTGAGGAAACAATTCTGAGCTGGTTCAGCCAAAGAGATAC | |
| | AACTGACAAGGGCCAGCAGTTGCGCAAGAATCAACAGCTGCAGAG | |
| | GTTCATCCAGTGGCTAAAAGAGGCAGAAGAGGAGTCATCTGAAGA | |
| | TGACTGACGCGCGCGAGATCTACGGGTGGCATCCCTGTGACCCCT | |
| | CCCCAGTGCCTCTCCTGGCCCTGGAAGTTGCCACTCCAGTGCCCA | |
| | CCAGCCTTGTCCTAATAAAATTAAGTTGCATCATTTTGTCTGACT | |
| | AGGTGTCCTTCTATAATATTATGGGGTGGAGGGGGGTGGTATGGA | |
| | GCAAGGGGCAAGTTGGGAAGACAACCTGTAGGGCCTGCGGGGTCT | |
| | ATTGGGAACCAAGCTGGAGTGCAGTGGCACAATCTTGGCTCACTG | |
| | CAATCTCCGCCTCCTGGGTTCAAGCGATTCTCCTGCCTCAGCCTC | |
| | CCGAGTTGTTGGGATTCCAGGCATGCATGACCAGGCTCAGCTAAT | |
| | TTTTGTTTTTTTGGTAGAGACGGGGTTTCACCATATTGGCCAGGC | |
| | TGGTCTCCAACTCCTAATCTCAGGTGATCTACCCACCTTGGCCTC | |
| | CCAAATTGCTGGGATTACAGGCGTGAACCACTGCTCCCTTCCCTG | |
| | TCCTTCTGATTTTGTAGGTAACCACGTGCGGACCGAGCGGCCGCA | |
| | GGAACCCCTAGTGATGGAGTTGGCCACTCCCTCTCTGCGCGCTCG | |
| | CTCGCTCACTGAGGCCGGGCGACCAAAGGTCGCCCGACGCCCGGG | |
| | CTTTGCCCGGGCGGCCTCAGTGAGCGAGCGAGCGCGCAGCTGCCT | |
| | GCAGG | |

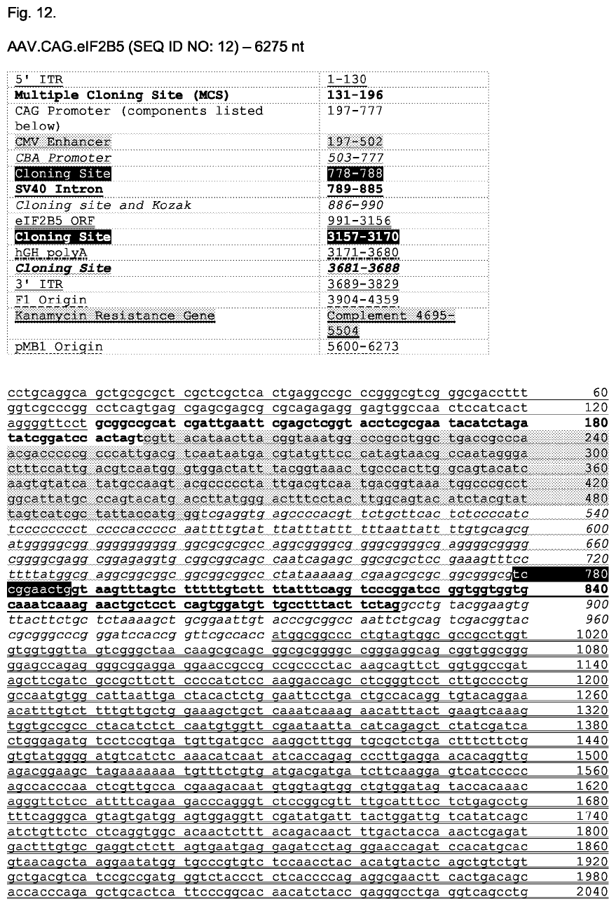

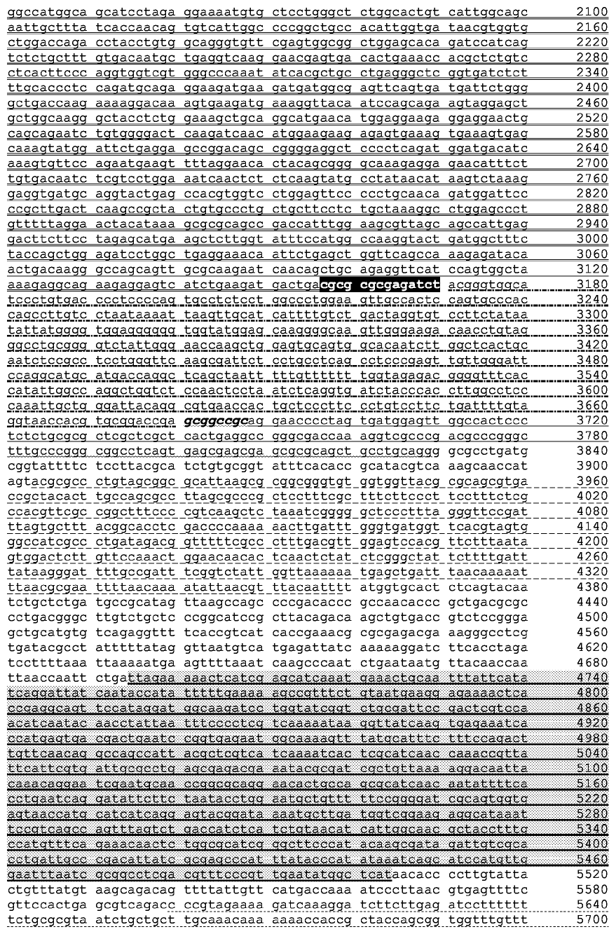

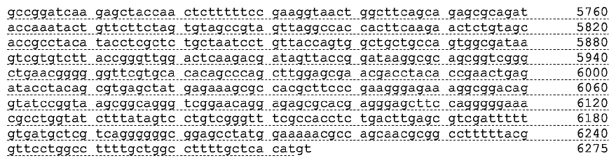

In some aspects, a nucleic acid of the disclosure comprises an AAV9 vector comprising a 5′ ITR, a promoter, an SV40 intron, the elF2B5 open reading frame, a polyA tail, and a 3′ ITR as illustrated in FIGS. 12-14, 16, and 19. In some particular aspects, the AAV is an ssAAV or an ssrAAV. In some aspects, a nucleic acid of the disclosure comprises a nucleotide sequence comprising from 5′ to 3′: a promoter, an SV40 intron, the elF2B5 open reading frame, and a polyA tail as set forth in any one of FIGS. 12-14 and 19. In some aspects, a nucleic acid of the disclosure comprises a nucleotide sequence comprising from 5′ to 3′: a 5′ ITR, a promoter, an SV40 intron, the elF2B5 open reading frame, a polyA tail, and a 3′ ITR as set forth in any one of FIGS. 12-14, 16, and 19. In some aspects, the nucleic acid may comprise additional elements, or some elements may be modified or eliminated. In some aspects, the nucleotide sequence of any of the 5′ ITR, promoter, SV40 intron, elF2B5 open reading frame, polyA tail, or 3′ ITR as illustrated in FIGS. 12-14, 16, and 19 may be a variant comprising 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence of any of the 5′ ITR, promoter, SV40 intron, elF2B5 open reading frame, polyA tail, or 3′ ITR sequences as illustrated in FIGS. 12-14, 16, and 19.

In some aspects, a nucleic acid of the disclosure comprises the nucleotide sequence set forth in any one of SEQ ID NOs: 12-14 and 18 (i.e., see FIGS. 12-14, 16, and 19). In various aspects, the nucleic acid is a variant of the nucleotide sequence set forth in any one of SEQ ID NOs: 12-14 and 18. In some aspects, the variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 12-14 and 18.

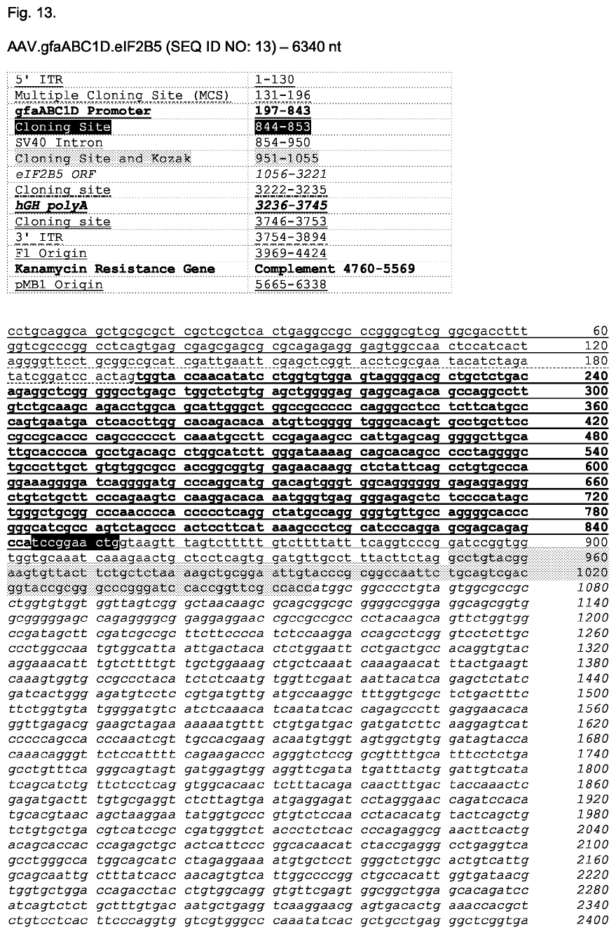

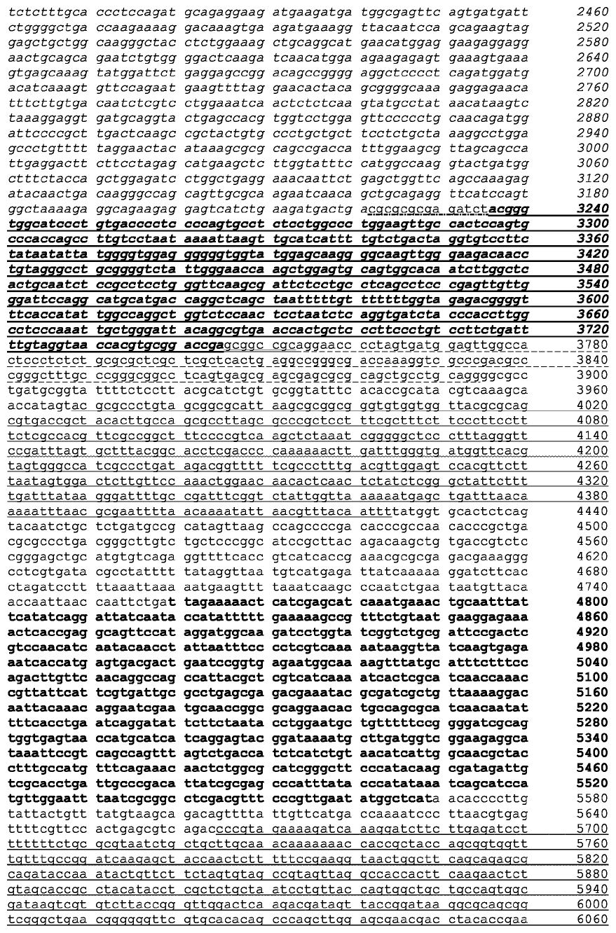

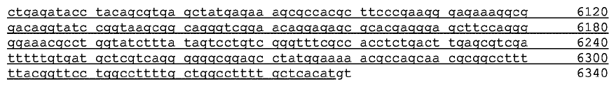

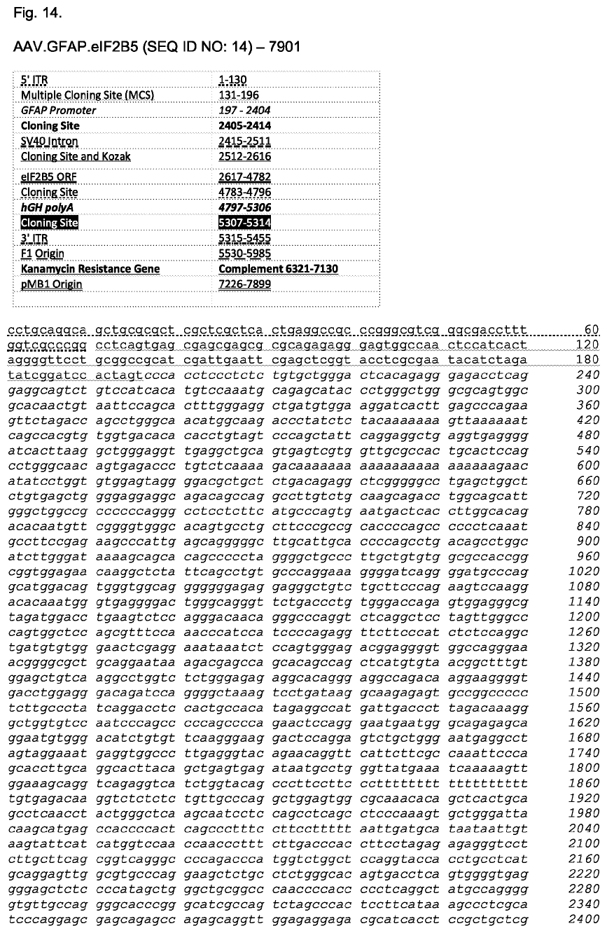

TABLE 4
Sequences of each of the AAV9 vectors comprising
the eIF2B5 transgene sequences.

| Sequence Name | Sequence (5′-3′) | SEQ ID NO: |
|---|---|---|
| AAV9.CAG.eIF2B5 | See FIG. 12. | 12 |
| (6275 bp) | | |
| AAV9.gfaABC(1)D.eIF2B5 | See FIG. 13. | 13 |
| (6340 bp) | | |
| AAV9.GFAP.eIF2B5 | See FIG. 14. | 14 |
| (7901 bp) | | |
| AAV9.gfa1405.eiF2B5 (7086 | See FIG. 21. | 18 |
| bp) | | |

A sequence index table (Table 5) is provided below for reference to sequences provided in the sequence listing.

TABLE 5
□Sequence index.

| SEQ | |
|---|---|
| ID NO: | Sequence Descriptor |
| 1 | Human EIF2B5 nucleotide |
| 2 | Human EIF2B5 polypeptide |
| 3 | CAG promoter |
| 4 | gfaABC1D promoter |
| 5 | GFAP promoter |
| 6 | CAG.EIF2B5 with various other elements but without 5′ and 3′ |
| | ITRs |
| 7 | gfaABC1D.EIF2B5 with various other elements but without 5′ |
| | and 3′ ITRs |
| 8 | GFAP.EIF2B5 with various other elements but without 5′ and 3′ |
| | ITRs |
| 9 | ITR-CAG.EIF2B5-ITR with various other elements and with 5′ |
| | and 3′ ITRs |
| 10 | ITR-gfaABC1D.EIF2B5-ITR with various other elements but |
| | with 5′ and 3′ ITRs |
| 11 | ITR-GFAP.EIF2B5-ITR with various other elements but with 5′ |
| | and 3′ ITRs |
| 12 | AAV9. CAG.eIF2B5 (6275 bp) |
| 13 | AAV9.gfaABC(1)D.eIF2B5 (6340 bp) |
| 14 | AAV9.GFAP.eIF2B5 (7901 bp) |
| 15 | gfa1405 promoter |
| 16 | gfa1405.EIF2B5 with various other elements but without 5′ and |
| | 3′ ITRs |
| 17 | gfa1405.EIF2B5 with various other elements and with 5′ and 3′ |
| | ITRs |
| 18 | AAV9.gfa1405.eiF2B5 (7086 bp) |

In some aspects, the disclosure includes a nanoparticle, extracellular vesicle, exosome, or vector comprising any of the nucleic acids of the disclosure or a combination of any one or more thereof for providing EIF2B5 gene replacement. In some aspects, one or more copies of these sequences are combined into a single nanoparticle, extracellular vesicle, exosome, or vector.

The disclosure therefore includes vectors comprising a nucleic acid of the disclosure or a combination of nucleic acids of the disclosure. Embodiments of the disclosure utilize vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein.

The disclosure provides a recombinant (r) AAV vector comprising the nucleic acid comprising a polynucleotide encoding the EIF2B5 protein for use in treating a subject comprising a mutation in the EIF2B5 gene. In some particular aspects, the AAV is an ssAAV or an ssrAAV. In some aspects, a nucleic acid of the disclosure comprises an AAV vector comprising the nucleotide sequence set forth in SEQ ID NO: 1 (Table 1). In some aspects, a nucleic acid of the disclosure comprises an AAV vector comprising the nucleotide sequence set forth in any one of SEQ ID NOs: 6-8 and 16 (Table 2). In some aspects, a nucleic acid of the disclosure comprises an AAV vector comprising the nucleotide sequence set forth in any one of SEQ ID NOs: 9-11 and 17 (Table 3). In various aspects, the nucleic acid is a variant of the nucleotide sequence set forth in any one of SEQ ID NOs: 1, 6-11, 16, and 17. In some aspects, the variant comprises 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in any one of SEQ ID NOs: 1, 6-11, 16, and 17.

AAV is unique in its safety profile, as the viral genome, once transduced into its carrier cell, remains stably expressed as an episomal DNA and only very rarely ever integrates into the host genome.

In some aspects, therefore, the disclosure utilizes AAV to deliver the EIF2B5 transgene, such as DNA encoding the EIF2B5 protein. As used herein, the term “AAV” is a standard abbreviation for adeno-associated virus. An “AAV vector” as used herein refers to a vector comprising one or more polynucleotides of interest (or transgenes) that are flanked by AAV terminal repeat sequences (ITRs).

Such AAV vectors can be replicated and packaged into infectious viral particles when present in a host cell that has been transfected with a vector encoding and expressing rep and cap gene products. AAV is a single-stranded replication-deficient DNA parvovirus that grows only in cells in which certain functions are provided by a co-infecting helper virus. The genome of AAV is about 4.7 kb in length including 145 nucleotide inverted terminal repeat (ITRs). There are multiple serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

There are multiple serotypes of AAV. In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh. 10, MyoAAV 1A, AAVMYO, or AAV-B1, AAV2/1, AAV2/8, AAV2/9, or any of their derivatives. Other types of rAAV variants, for example rAAV with capsid mutations, are also included in the disclosure. See, for example, Marsic et al., Molecular Therapy 22 (11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

The nucleotide sequences of the genomes of the AAV serotypes are known. For example, the complete genome of AAV1 is provided in GenBank Accession No. NC_002077; the complete genome of AAV2 is provided in GenBank Accession No. NC_001401 and Srivastava et al., J. Virol., 45:555-564 {1983); the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV6 is provided in GenBank Accession No. NC_00 1862; at least portions of AAV7 and AAV8 genomes are provided in GenBank Accession Nos. AX753246 and AX753249, respectively (see also U.S. Pat. Nos. 7,282,199 and 7,790,449 relating to AAV8); the AAV9 genome is provided in Gao et al., J. Virol., 78:6381-6388 (2004); the AAV10 genome is provided in Mol. Ther., 13 (1): 67-76 (2006); and the AAV11 genome is provided in Virology, 330 (2): 375-383 (2004). Information regarding MyoAAV 1A is provided by Tabebordbar et al. (Cell 184 (19): 4919-38 (2021)). Information regarding AAVMYO is provided by Weinmann et al. (Nature Communications 11:5432 (2020); doi.org/10.1038/s41467-020-19230). The genomes of AAV12, AAV13, AAVanc80, AAVrh.74, AAVrh.8, AAVrh. 10, and AAV-B1 also are known in the art. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

Cis-acting sequences directing viral DNA replication (rep), encapsidation/packaging and host cell chromo-some integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus, making cold preservation of AAV less critical. AAV may be lyophilized and AAV-infected cells are not resistant to superinfection.

In some aspects, the AAV lacks rep and cap genes. In some aspects, the AAV is a recombinant linear AAV (rAAV), a single-stranded AAV, or a recombinant self-complementary AAV (scAAV). The self-complementary (sc) technology allows for binding of the single-stranded viral DNA genome onto itself, thereby priming second strand DNA synthesis. This sc element both quickens and strengthens gene expression relative to constructs lacking the sc element. In some particular aspects, the AAV is an ssAAV or an ssrAAV.

Advances in AAV vectors have led to safer and more efficient viral vehicles to deliver therapeutic transgenes in a single injection, and gene therapy is now a favorable therapeutic intervention for monogenic diseases. AAV vectors can provide long-term expression of gene products in post-mitotic target tissues. Thus, current AAV-based strategies may only require one-time vector administration.

Recombinant AAV genomes of the disclosure comprise one or more AAV ITRs flanking a polynucleotide encoding, for example, one or more EIF2B5 polynucleotides, including any one or more of the sequences set out in SEQ ID NOs: 1 and 6-11. Provided herein are rAAV, each comprising one or more EIF2B5 genes. An rAAV comprising one or more EIF2B5 genes can encode one, two, three, four, five, six, seven or eight EIF2B5 proteins.

In some aspects, therefore, the viral vector is an AAV, such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAV10 (i.e., an AAV containing AAV10 ITRs and AAV10 capsid proteins), AAV11 (i.e., an AAV containing AAV11 ITRs and AAV11 capsid proteins), AAV12 (i.e., an AAV containing AAV12 ITRs and AAV12 capsid proteins), AAV13 (i.e., an AAV containing AAV13 ITRs and AAV13 capsid proteins), AAVanc80 (i.e., an AAV containing AAVanc80 ITRs and AAVanc80 capsid proteins), AAVrh.74 (i.e., an AAV containing AAVrh.74 ITRs and AAVrh.74 capsid proteins), AAVrh.8 (i.e., an AAV containing AAVrh.8 ITRs and AAVrh.8 capsid proteins), AAVrh. 10 (i.e., an AAV containing AAVrh. 10 ITRs and AAVrh. 10 capsid proteins), MyoAAV 1A, AAVMYO, or AAV-B1, or pseudotyped AAV, such as AAV2/1, AAV2/8, or AAV2/9, or AAVMYO, or any of their derivatives.

In various aspects, the AAV is AAV9. AAV9 has become the most widely used vector for muscular and/or neurological indications with an established safety profile in the clinic. Intrathecal administration of AAV9 permits dissemination of transgenes throughout the nervous system and is currently approved by FDA for spinal muscular atrophy (SMA, NCT03381729), and in trials for the treatment of neuronal ceroid lipofuscinosis 3 (CLN3, NCT03770572), CLN6 (NCT02725580), giant axonal neuropathy (GAN, NCT02362438), mucopolysaccharidoses types 3A (NCT02716246) and 3B (NCT03315182), and exon 2 duplications in the DMD gene (NCT04240314). Such features make AAV9 an ideal gene delivery method for treatment of genetic disorders, such as mutations in EIF2B5, which result in white matter abnormalities in the central nervous system. It has been shown that AAV9 can also target Schwann cells, and other peripheral neuropathies. More importantly, AAV9 was reported to transduce Schwann cells in large animals and non-human primates, indicating that it is a desirable viral vector for clinical applications requiring delivery of therapeutic genes into the human Schwann cells. Finally, data from studies in other models of CNS disease show that an AAV9 vector efficiently transfects CNS (Lukashchuk et al., Molecular Therapy 3:15055, 2016; doi.org/10.1038/mtm.2015.55).

DNA plasmids of the disclosure comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh. 10, MyoAAV 1A, AAVMYO, or AAV-B1. In some aspects, AAV DNA in the rAAV genomes is from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh. 10, MyoAAV 1A, AAVMYO, or AAV-B1. Other types of rAAV variants, for example rAAV with capsid mutations, are also included in the disclosure. See, for example, Marsic et al., Molecular Therapy 22 (11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety. An “AAV virion” or “AAV viral particle” or □AAV particle □or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13, AAVrh.74, AAVrh.8, or AAVrh. 10, MyoAAV 1A, AAVMYO, or AAV-B1, AAVAnc80, AAV7m8, AAV2/1, AAV2/8, or AAV2/9 and their derivatives. In some aspects, AAV DNA in the rAAV genomes is from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVrh.74, AAVrh.8, or AAVrh. 10, MyoAAV 1A, AAVMYO, or AAV-B1, AAVAnc80, AAV7m8, AAV2/1, AAV2/8, or AAV2/9 and their derivatives. Other types of rAAV variants, including those for example with capsid mutations, are also included in the disclosure. Such variants include, but are not limited to, MyoAAV or AAVMYO, and other variants as described, for example, in Marsic et al., Molecular Therapy 22 (11): 1900-1909 (2014); Weismann, J., et al., Nat Commun 11 (1): 5432 (2020) and Tabebordbar, M. et al., Cell 184 (19): 4919-4938 e22 (2021), which are incorporated for use herein by reference in their entirety. As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some embodiments, the viral vector is a pseudotyped AAV, containing ITRs from one AAV serotype and capsid proteins from a different AAV serotype. In some embodiments, the pseudo-typed AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is AAV2/1 (i.e., an AAV containing AAV2 ITRs and AAV1 capsid proteins).

In some embodiments, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAV11, AAV12, AAV13, AAVrh.74, AAVrh.8, or AAVrh. 10, MyoAAV 1A, AAVMYO, or AAV-B1, AAVAnc80, AAV7m8, AAV2/1, AAV2/8, or AAV2/9 and their derivatives. Other types of rAAV variants, for example rAAV with capsid mutations, are also contemplated. See, for example, Marsic et al., Molecular Therapy, 22 (11): 1900-1909 (2014). The nucleotide sequences of the genomes of various AAV serotypes are known in the art.

Multiple studies have demonstrated long-term (>1.5 years) recombinant AAV-mediated protein expression. See, Clark et al., Hum Gene Ther, 8:659-669 (1997) 32; Kessler et al., Proc Nat. Acad Sc. USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001).

Recombinant AAV genomes, in various aspects, comprise nucleic acids of the disclosure and one or more AAV ITRs flanking the nucleic acid. AAV DNA in the rAAV genomes may be from any AAV serotype for which a recombinant virus can be derived including, but not limited to, AAV serotypes (e.g., AAV1, AAV2, AAV3, AAV4, AAV5, AAV6, AAV7, AAV8, AAV9, AAV10, AAV11, AAV12, AAV13, AAVrh.74, AAVrh.8, or AAVrh. 10, MyoAAV 1A, AAVMYO, or AAV-B1, AAVAnc80, AAV7m8, AAV2/1, AAV2/8, or AAV2/9 and their derivatives). Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014)²⁹. As noted in the Background section above, the nucleotide sequences of the genomes of various AAV serotypes are known in the art.

The provided recombinant AAV (i.e., infectious encapsidated rAAV particles) comprise a rAAV genome. The term □rAAV genome□refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a □gene cassette. □In exemplary embodiments, the genomes of both rAAV lack AAV rep and cap DNA, that is, there is no AAV rep or cap DNA between the ITRs of the genomes.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13, AAVrh.74, AAVrh.8, or AAVrh. 10, MyoAAV 1A, AAVMYO, or AAV-B1, AAVAnc80, AAV7m8, AAV2/1, AAV2/8, or AAV2/9 and their derivatives. Production of pseudotyped rAAV is disclosed in, for example, WO 01/83692 which is incorporated by reference herein in its entirety.

Recombinant AAV genomes of the disclosure comprise one or more AAV ITRs flanking a polynucleotide encoding, for example, one or more EIF2B5. Embodiments of the disclosure, therefore include a rAAV genome comprising a nucleic acid comprising a nucleotide sequence set out in any one of SEQ ID NOs: 1, 6-11, 16, and 17, or a nucleotide sequence comprising at least or about or at least about 70%, about 75%, about 80%, about 85%, about 90%, about 91%, about 92%, about 93%, about 94%, about 95%, about 96%, about 97%, about 98%, or about 99% identity to a sequence set out in any one of SEQ ID NOs: 1, 6-11, 16, and 17.

Embodiments of the disclosure, include a nucleic acid comprising a rAAV genome comprising a nucleotide sequence set out in any one of SEQ ID NOs: 12-14 and 18, or a nucleotide sequence comprising at least or about or at least about 70%, about 75%, about 80%, about 85%, about 90%, about 91%, about 92%, about 93%, about 94%, about 95%, about 96%, about 97%, about 98%, or about 99% identity to a sequence set out in any one of SEQ ID NOs: 12-14 and 18.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing, addition of synthetic linkers containing restriction endonuclease cleavage sites⁴¹ or by direct, blunt-end ligation. The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129. Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., Mol. Cell. Biol., Oct; 8 (10): 3988-96 (1988); Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production. The production and use of self-complementary (sc) rAAV are specifically contemplated and exemplified.

The disclosure thus provides packaging cells that produce infectious rAAV. In one embodiment, packaging cells are stably transformed cancer cells, such as Hela cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In some aspects, rAAV is purified by methods standard in the art, such as by column chromatography or cesium chloride gradients. Methods for purifying rAAV vectors from helper virus are known in the art and include methods disclosed in, for example, Clark et al., Hum. Gene Ther., 10 (6): 1031-1039 (1999); Schenpp and Clark, Methods Mol. Med., 69 427-443 (2002); U.S. Pat. No. 6,566,118 and WO 98/09657.

Compositions comprising the nucleic acids and viral vectors of the disclosure are provided. Compositions comprising delivery vehicles (such as rAAV) described herein are provided. In various aspects, such compositions also comprise a pharmaceutically acceptable carrier. In some aspects, a pharmaceutically acceptable carrier is a diluent, excipient, or buffer. The compositions may also comprise other ingredients, such as adjuvants.

Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include, but are not limited to, a dosage of about 1×10⁷, about 1×10⁸, about 1×10⁹, about 5×10⁹, about 6×10⁹, about 7×10⁹, about 8×10⁹, about 9×10⁹, about 1×10¹⁰, about 2×10₁₀, about 3×10₁₀, about 4×10¹⁰, about 5×10¹⁰, about 6×10¹⁰, about 7×10¹⁰, about 8×10¹⁰, about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹, about 1×10¹², about 2×10¹², about 3×10¹², about 4×10¹², about 5×10¹², about 6×10¹², about 7×10¹², about 8×10¹², about 9×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5×10¹³, about 6×10¹³, about 7×10¹³, about 8×10¹³, about 9×10¹³, about 1×10¹⁴, about 2×10¹⁴, about 3×10¹⁴, about 4×10¹⁴, about 5×10¹⁴, about 1×10¹⁵, to about 1×10¹⁶, or more total viral genomes.

Dosages of about 1×10⁹ to about 1×10¹⁰, about 5×10⁹ to about 5×10¹⁰, about 1×10¹⁰ to about 1×10¹¹, about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, about 1×10¹³ to about 1×10¹⁵ vg and about 6×10¹³ to about 1.0×10¹⁴ vg are also contemplated. One dose exemplified herein is 1×10¹³ vg administered via intrathecal, intracerebroventricular, intracerebral, intravenous, intracisternal, or aerosol delivery.

Dosages of rAAV to be administered also, in various aspects, are expressed in units of vg/kg. Such dosages include, but are not limited to, a dosage of about 1×10⁷ vg/kg, about 1×10⁸ vg/kg, about 1×10⁹ vg/kg, about 5×10⁹ vg/kg, about 6×10⁹ vg/kg, about 7×10⁹ vg/kg, about 8×10⁹ vg/kg, about 9×10⁹ vg/kg, about 1×10¹⁰ vg/kg, about 2×10¹⁰ vg/kg, about 3×10¹⁰ vg/kg, about 4×10¹⁰ vg/kg, about 5×10¹⁰ vg/kg, about 1×10¹¹ vg/kg, about 5×10¹¹ vg/kg, about 1×10¹² vg/kg, about 2×10¹² vg/kg, about 3×10¹² vg/kg, about 4×10¹² vg/kg, about 5×10¹² vg/kg, about 6×10¹² vg/kg, about 7×10¹² vg/kg, about 8×10¹² vg/kg, about 9×10¹² vg/kg, about 1×10¹³ vg/kg, about 1.1×10¹³ vg/kg, about 1.2×10¹³ vg/kg, about 1.3×10¹³ vg/kg, about 1.5×10¹³ vg/kg, about 2×10¹³ vg/kg, about 2.5×10¹³ vg/kg, about 3×10¹³ vg/kg, about 3.5×10¹³ vg/kg, about 4×10¹³ vg/kg, about 4.5×10¹³ vg/kg, about 5×10¹³ vg/kg, about 6×10¹³ vg/kg, about 7×10¹³ vg/kg, about 8×10¹³ vg/kg, about 9×10¹³ vg/kg, about 1×10¹⁴ vg/kg, about 2×10¹⁴ vg/kg, about 3×10¹⁴ vg/kg, about 4×10¹⁴ vg/kg, about 5×10¹⁴ vg/kg, about 1×10¹⁵ vg/kg, or about 1×10¹⁶ vg/kg.

Dosages of about 1×10⁹ vg/kg to about 1×10¹⁰ vg/kg, about 5×10⁹ vg/kg to about 5×10¹⁰ vg/kg, about 1×10¹⁰ vg/kg to about 1×10¹¹ vg/kg, about 1×10¹¹ vg/kg to about 1×10¹⁵ vg/kg, about 1×10¹² vg/kg to about 1×10¹⁵ vg/kg, about 1×10¹² vg/kg to about 1×10¹⁴ vg/kg, about 1×10¹³ vg/kg to about 2×10¹⁴ vg/kg, about 1×10¹³ vg/kg to about 1×10¹⁵ vg/kg and about 6×10¹³ vg/kg to about 1.0×10¹⁴ vg/kg are also included in various aspects. Some doses exemplified herein are about 1.5×10¹¹ vg/kg or about 3×10¹³ vg/kg administered via intrathecal, intracerebroventricular, intracerebral, intravenous, intracisternal, or aerosol delivery.

Transduction or transfection of cells with rAAV of the disclosure results in sustained expression of the EIF2B5 gene/protein. As used herein, the terms □transduction□ and □transfection□are used interchangeably. The term □transduction □or □transfection □is used to refer to, as an example, the administration/delivery of the EIF2B5 gene to a target cell either in vivo or in vitro, via a replication-deficient rAAV described herein resulting in the expression of the EIF2B5 gene/protein by the target cell. The disclosure thus provides methods of administering/delivering rAAV which express the EIF2B5 gene to a cell or to a subject. In some aspects, the subject is a mammal. In some aspects, the mammal is a human. These methods include transducing cells and tissues (including, but not limited to, astrocytes, neurons, glia, peripheral motor neurons, sensory motor neurons, neurons, Schwann cells, and other tissues or organs, such as muscle, liver and brain) with one or more rAAV described herein. Transduction may be carried out with gene cassettes comprising cell-specific control elements.

Methods of transducing a target cell, such as an astrocyte, with a delivery vehicle (such as a nanoparticle, extracellular vesicle, exosome, or vector (e.g., rAAV)), in vivo or in vitro, are provided. The in vivo methods comprise the step of administering an effective dose, or effective multiple doses, of a composition comprising a delivery vehicle (such as rAAV) to an animal (including a human subject or patient) in need thereof. If the dose is administered prior to development of a disorder/disease, the administration is prophylactic. If the dose is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Thus, methods are provided of administering an effective dose (or doses, administered essentially simultaneously or doses given at intervals) of rAAV described herein to a subject in need thereof.

Provided herein are medicaments and methods for treating, ameliorating, or preventing diseases associated with a mutant EIF2B5 gene or aberrant EIF2B5 gene expression. Molecular, biochemical, histological, and functional outcome measures demonstrate the therapeutic efficacy of the methods. The level of human EIF2B5 transcript in animals and/or in humans can be confirmed by RT-PCR and/or RNAseq. The EIF2B5 protein expression level in tissues and organs of interest can be assessed using western blotting. EIF2B5 localization can be confirmed by immunohistochemistry. To assess efficacy of potential treatment in mice, measurements of function can be performed using various functional outcome measures, including, but not limited to, rotarod testing and other functional testing methods for leukodystrophies known in the art. In patients, a variety of functional outcome measures may be used to assess successful treatment including, but not limited to, gait analysis, MRI/DTI, seizure monitoring (EEG), and other testing methods for leukodystrophies known in the art. Additionally, see, for example, Parikh et al., Mol Genet Metab 2015; 114 (4): 501-515.

In the methods of the disclosure, expression of the EIF2B5 protein is increased by at least 10, at least 20, at least 30, at least 40, at least 50, at least 60, at least 70, at least 80, at least 90, at least 95, at least 98 percent, at least 99 percent, or 100 percent.

Combination therapies are also contemplated by the disclosure. Combination as used herein includes both simultaneous treatment and sequential treatments. Combinations of methods of the disclosure with standard medical treatments are specifically contemplated, as are combinations with novel therapies. In some embodiments, the combination therapy comprises administering an immunosuppressing agent in combination with the gene therapy disclosed herein.

The immunosuppressing agent may be administered before or after the onset of an immune response to the rAAV in the subject after administration of the gene therapy. In addition, the immunosuppressing agent may be administered simultaneously with the gene therapy or the protein replacement therapy. The immune response in a subject includes an adverse immune response or an inflammatory response following or caused by the administration of rAAV to the subject. The immune response may be the production of antibodies in the subject in response to the administered rAAV.

Exemplary immunosuppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors, biologics such as monoclonal antibodies or fusion proteins and polypeptides, and di peptide boronic acid molecules, such as Bortezomib.

The immunosuppressing agent may be an anti-inflammatory steroid, which is a steroid that decreases inflammation and suppresses or modulates the immune system of the subject. Exemplary anti-inflammatory steroid are glucocorticoids such as prednisolone, betamethasone, dexamethasone, methotrexate, hydrocortisone, methylprednisolone, deflazacort, budesonide or prednisone. Janus kinase inhibitors are inhibitors of the JAK/STAT signaling pathway by targeting one or more of the Janus kinase family of enzymes. Exemplary janus kinase inhibitors include tofacitinib, baricitinib, upadacitinib, peficitinib, and oclacitinib.

Calcineurin inhibitors bind to cyclophilin and inhibits the activity of calcineurin Exemplary calcineurine inhibitors includes cyclosporine, tacrolimus and picecrolimus.

mTOR inhibitors reduce or inhibit the serine/threonine-specific protein kinase mTOR. Exemplary mTOR inhibitors include rapamycin (also known as sirolimus), everolimus, and temsirolimus.

The immunosuppressing agents include immune suppressing macrolides. The term □immune suppressing macrolides □refer to macrolide agents that suppresses or modulates the immune system of the subject. A macrolide is a class of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and rapamycin (also known as sirolimus).

Purine analogs block nucleotide synthesis and include IMDH inhibitors. Exemplary purine analogs include azathioprine, mycophenolate such as mycophenolate acid or mycophenolate mofetil and lefunomide.

Exemplary immunosuppressing biologics include abatacept, adalimumab, anakinra, certolizumab, etanercept, golimumab, infliximab, ixekizumab, natalizumab, rituximab, secukinumab, tocilizumab, ustekinenumab, vedolizumab, basiliximab, belatacep, and daclizumab.

In particular, the immunosuppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab or ofatumumab.

Additional examples of immuosuppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab and visilizumab, anti-CD52 antibodies such as alemtuzumab.

One exemplary combination therapy is the delivery of rapamycin and rituximab prior to, or contemporaneous with, delivery of the AAV vector. Another exemplary combination therapy is the delivery of rapamycin, rituximab, and a corticosteroid, such as prednisone.

Administration of an effective dose of a nucleic acid, nanoparticle, extracellular vesicle, exosome, viral vector, or composition of the disclosure may be by routes standard in the art including, but not limited to, intrathecal, intracerebral, intracerebroventricular, intracisternal, intramuscular, parenteral, intravascular, intravenous, oral, buccal, nasal, pulmonary, intracranial, intraosseous, intraocular, rectal, or vaginal. In various aspects, an effective dose is delivered by a combination of routes. For example, in various aspects, an effective dose is delivered intrathecally, intracerebrally, intracerebroventricularly, intravenously, intracisternally, and/or intramuscularly, or intrathecally and/or intravenously and/or intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the EIF2B5 gene.

In particular, actual administration of delivery vehicle (such as rAAV) may be accomplished by using any physical method that will transport the delivery vehicle (such as rAAV) into a target cell (i.e., an astrocyte) of a subject. Administration includes, but is not limited to, injection into the cerebrospinal fluid (CSF) (intrathecally), injection intracerebroventricularly, injection intracerebrally, injection into the bloodstream and/or directly into the nervous system, injection intracisternally (or via intracisterna magna (ICM)), or nasally. Simply resuspending a rAAV in phosphate buffered saline has been demonstrated to be sufficient to provide a vehicle useful for tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations for intrathecal injection or as aerosol formulations for inhalation. Numerous formulations for intrathecal, intracerebroventricular, intracerebral, or intracisternal injection have been previously developed and can be used in the practice of the methods of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, suMPZ or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure also provides kits for use in the treatment of a disease or disorder described herein. Such kits include at least a first sterile composition comprising any of the nucleic acids described herein above or any of the viral vectors described herein above in a pharmaceutically acceptable carrier. Another component is optionally a second therapeutic agent for the treatment of the disorder along with suitable container and vehicles for administrations of the therapeutic compositions. The kits optionally comprise solutions or buffers for suspending, diluting or effecting the delivery of the first and second compositions.

In one embodiment, such a kit includes the nucleic acids or vectors in a diluent packaged in a container such as a sealed bottle or vessel, with a label affixed to the container or included in the package that describes use of the nucleic acids or vectors. In one embodiment, the diluent is in a container such that the amount of headspace in the container (e.g., the amount of air between the liquid formulation and the top of the container) is very small. Preferably, the amount of headspace is negligible (i.e., almost none).

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, stabilizers, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

In some aspects, the kit comprises a label and/or instructions that describes use of the reagents provided in the kit. The kits also optionally comprise catheters, syringes or other delivering devices for the delivery of one or more of the compositions used in the methods described herein.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

All publications, patents and patent applications cited in this specification are herein incorporated by reference as if each individual publication or patent application were specifically and individually indicated to be incorporated by reference in its entirety to the extent that it is not inconsistent with the disclosure.

It is understood that the examples and embodiments described herein are for illustrative purposes only and that various modifications or changes in light thereof will be suggested to persons skilled in the art and are to be included within the spirit and purview of this application and scope of the appended claims. Thus, the following examples are provided by way of illustration and not limitation.

### EXAMPLES

### Example 1

### Gene Therapy Constructs Encoding EIF2B5 Protein and Materials and Methods

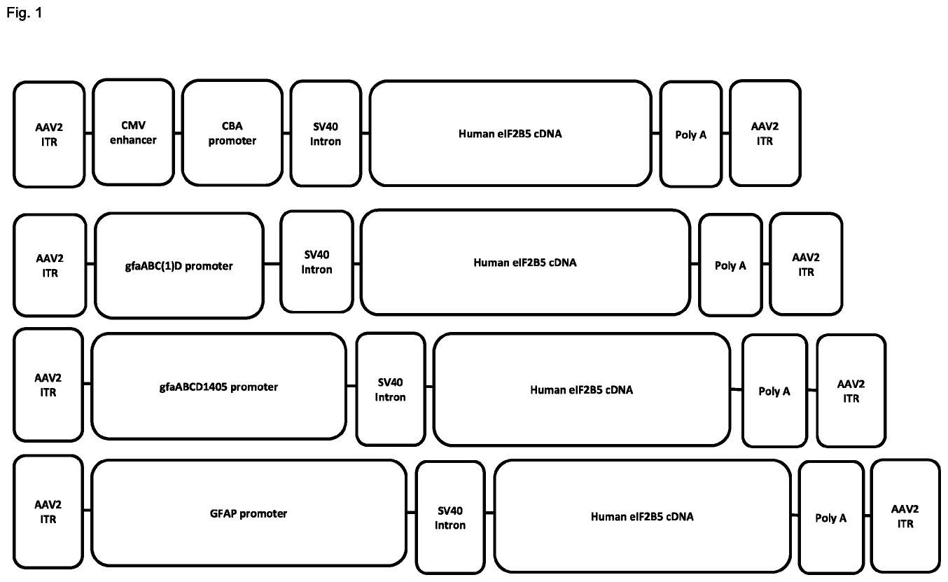

Initially, three AAV genome vector constructs encoding EIF2B5 were generated. These constructs include promoters targeting astrocytes: a ubiquitous promoter comprising a CBA promoter and a CMV enhancer, referred to as the CAG promoter (SEQ ID NO: 3), the full-length glial fibrillary acidic protein (GFAP) promoter (SEQ ID NO: 5), and the truncated version of the GFAP promoter, i.e., the gfaABC(1)D promoter (SEQ ID NO: 4). The gfaABC(1)D promoter is a compact GFAP promoter derived from the conventional ˜2.2 kb human GFAP promoter. The other key components of the vectors include AAV9 capsid for efficient targeting of the CNS, AAV2 inverted terminal repeats (ITRs) creating a single-stranded construct with a larger packaging capacity (for the gene of interest), the human elF2B5 coding sequence, and a post-transcriptional polyadenylation (polyA) sequence (FIG. 1).

Each of these sequences comprising the full-length transcript of EIF2B5 cDNA under the control of a CAG promoter (SEQ ID NO: 3), a gfaABC(1)D promoter (SEQ ID NO: 4), or a GFAP promoter (SEQ ID NO: 5) and various other components as shown in FIGS. 12-14. Each of these constructs was designed to test the restoration of EIF2B5 expression. The specific sequences for each of the three constructs are provided in Tables 1-3 and are shown in FIGS. 12-14.

GFAP, gfaABC(1)D, and CBA promoters were each cloned into a plasmid with elF2B5. GFAP (2.2 kb) and elF2B5 (2.2 kb), and a truncated, gfABC(1)D promoter of only 681 bp were evaluated. Each construct was sequenced and mRNA and protein levels of elF2B5 were evaluated in vitro. Cloning, sequencing, and in vitro expression analysis were carried out.

### Materials and Methods

### Vector Production.

Recombinant AAV (rAAV) vectors were manufactured at Andelyn Biosciences using a calcium phosphate-mediated triple transfection in adherent HEK293 cells followed by purification. Briefly, harvested media was filtered and concentrated then purified by gradient ultracentrifugation followed by ion exchange chromatography. Vectors were formulated in 20 mM Tris (pH8.0), 1 mM MgCl₂ and 200 mM NaCl and 0.001% Pluronic F68 and sterile filtered. The AAV production process was developed using methods described by Rabinowotiz et al. (J. Virol. 2002 76 (2): 791-801; doi: 10.1128/jvi.76.2.791-801.2002. Physical titer determination was based on degradation of non-encapsidated DNA following digestion of viral capsids and determined by ddPCR.

### Cell Lines.

Human embryonic kidney 293 (HEK293) cells (ATCC cat #CRL-1573) were used to confirm transfection of plasmid DNA.

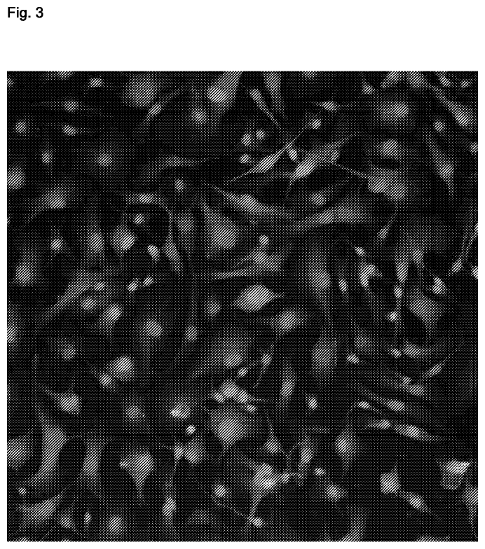

To establish an astrocyte cell line as a relevant in vitro system to study disease mechanisms and for testing the AAV constructs of the disclosure, a skin biopsy was performed on a patient with confirmed elF2B5 VWM. A fibroblast culture was established from a skin biopsy from the patient, and the fibroblasts were converted to astrocytes, the primary cell type of interest (FIG. 3).

### In Vitro GFP Expression.

90% confluent HEK293 cells in a 6-well plate were transfected with 2.5 ug of plasmid DNA using Lipofectamine 3000 following the manufacturer's protocol. 72 hours post transfection, the cells were imaged and harvested, and cell pellets were frozen at −80C. One well of untransfected cells was also harvested at the same time as a negative control.

RNA was isolated from the cells using Trizol, DNAsel treated, and cDNA was generated using the Qiagen RT2 First Strand synthesis kit. The relative GFP mRNA levels were quantified via qPCR using the comparative CT method with SYBR green and primers specific to GFP, and primers specific to beta actin as the endogenous control.

### Mice and Source of Mice.

### Wild-Type (WT) Mice.

Neonatal (days 1-3 post-natal) C57BL/6 mice were used in various experiments.

### R191H Vwm Mice.

R191H VWM mice, as described in Wong et al. (eLife. 2019; 8: e42940; doi: 10.7554/eLife.42940) are used as an animal model for VWM. The mutation described corresponds to the human G584A mutation, Arg195H, a Cree leukoencephalopathy, as described by Fogli et al. (Annals of Neurology 52 (4): 506-10, 2002); doi.org/10.1002/ana. 10339), which is one of the most rapidly progressing leukoencephalopathies, with a disease onset of 3-9 months, and 100% death by 21 months. This mouse model has about an 8-month survival with various reported symptoms as follows: motor deficits at 5 months, MBP expression decreased by 28 days, axon changes at 7 months, an increase in astrocyte number from P14 and increased with disease progression, and GFAPδ protein levels were increased in forebrain lysates.

### Icv Injections.

All animal procedure were approved by Nationwide Children's Hospital Institutional Animal Care and Use Committee (IACUC). Neonatal (days 1-3 post-natal) mice (C57BL/6) were cryo-anesthetized (˜2 min) prior to intracerebroventricular (ICV) injections. ICV injections were performed with a Hamilton syringe (Cal7635-01) and 33GA 30·beveled needles (Hamilton, 7803-05) into the left hemisphere at 2/5 of the distance from the lambda suture to the eye. Neonates were injected with 7.50E+10 vg of ssAAV9 vectors encoding GFP under the GFAP, CAG and the truncated variant gfaABC(1)D (GFAP) promoters.

Evaluation of GFP Biodistribution in Animal Tissues after AAV9 ICV Injections.

Four or eight weeks after ICV injections, mice were terminally anesthetized with Ketamine/Xylazine (100/10 mg/kg i.p.) and transcardially perfused with ice-cold 0.9% heparinized saline. Tissues were dissected and post-fixed in 4% PFA in PBS for 12 hr. After fixation, the right brain hemisphere was cryoprotected in 30% sucrose in PBS at 4° C. for 3 days. All samples were embedded and frozen in OST compound (Tissue Plus, Fisher). Sagittal sections were cut at 25 mm thickness on a cryostat (1950 LEICA). Free-floating sections were washed in PBS and incubated with DAPI solution in PBS for 1 min at RT. To retrieve antigen from PFA fixed tissues, slices were treated with 0.1% Sodium borohydride in 1× PBS 15 min at RT. For immunohistochemical analysis of the GFP colocalization with specific cellular markers, all the slices were blocked and permeabilized in 10% normal goat serum in 1×PBS with 0.3% Triton (PBST) for 1 hr at RT followed by overnight incubation in fresh PBST with chicken anti-GFAP (AbCam, 1:300) and rabbit anti-NeuN (Cell Signaling, 1:500) at 4C. Sections were washed in 1×PBS and incubated in PBST with Donkey anti-chicken Cy5 (Jackson ImmunoResearch, 1:500) and Donkey anti-Rabbit Alexa Fluor 568 (Thermo Fisher Scientific, 1:500) secondary antibodies in PBST with 10% normal donkey serum for 1 hr at RT. Sections were washed in 1×PBS and mounted on slides in ProLong Gold antifade reagent (Thermo Fisher Scientific). Images were acquired using a Nikon Ti2E fluorescent microscope and analyzed using NIS-Elements software (Nikon) and Prism (GraphPad). The percentage of GFP distribution was evaluated within the area covered by GFP and DAPI on each section. The intensity of GFP signal was evaluated within all GFP positive area.

### Protein Extraction and Western Blotting Analysis.

Protein extraction and Western blotting analysis were carried out after mice were treated. Total protein was extracted from selected animal tissues using tissue protein extraction reagent (T-PER Tissue Protein Extraction Reagent; 78510; Thermo Scientific) and 1 tablet of protease inhibitor (Pierce Protease Inhibitor Tablets; A32953; Thermo Scientific) per 10 mL of extraction reagent. Steel bead was added to sample and extraction reagent, and samples were homogenized using TissueLyser II. Steel bead was then removed, and samples were sonicated twice for 15 seconds.

Total protein extracted was quantified using the DC Protein Assay (Bio-Rad). Twenty-five microgram samples were separated on 4-12% SDS-PAGE and transferred to nitrocellulose membrane using the wet transfer system. Ponceau staining was performed and membranes were subsequently washed in PBS supplemented with 0.1% Tween three times, 10 min each. Membranes were blocked for 1 hour at RT in Pierce Protein Free Blocking Buffer. Nitrocellulose membranes were then incubated with the following antibodies overnight at 4C: mouse monoclonal antibody to GAPDH (1:5000 in Pierce Protein Free Blocking Buffer, 274102; Synaptic Systems); chicken polyclonal antibody to GFP (1:5000 in Pierce Protein Free Blocking Buffer, ab13970; AbCam). Membranes were then washed three times, 10 min each with PBST buffer.

Membranes were then incubated for 1 hour at RT with the following antibodies: goat antibody to chicken AF 488 (1:1000 in Pierce Protein Free Blocking Buffer, ab150169; AbCam); donkey antibody to mouse AF 568 (1:1000 in Pierce Protein Free Blocking Buffer, A 10037; Invitrogen). Membranes were then washed three times, 10 min each with PBST buffer.

Blots were subsequently imaged using blot imager. Quantification of bands was performed using Bio-Rad ImageLab.

Protein Extraction and Western Blotting Analysis after Transfection.

Protein extraction and Western blotting analysis were carried out after the 72-hours post-transfection cells were collected, washed in PBS, centrifuged, and flash frozen. Total protein was extracted from 1-2 million HEK293T cells by first thawing the cell pellets on ice for 15 minutes, and then using 30 μL of RIPA buffer per pellet (Pierce, RIPA lysis and Extraction Buffer 89901; Thermo Scientific) and 1 tablet of protease inhibitor (Pierce Protease Inhibitor Tablets; A32953; Thermo Scientific) per 10 mL of extraction reagent. Samples were gently homogenized using pipette, lysed on ice for 15 minutes at 4C, briefly sonicated, and then centrifuged at 10,000 g for 10 minutes. The supernatant was transferred to a new tube, and total protein was quantified.

Total protein extracted was quantified using the DC Protein Assay (Bio-Rad). Fifty microgram samples were separated on 4-12% SDS-PAGE and transferred to nitrocellulose membrane using the wet transfer system. Ponceau staining was performed and membranes were subsequently washed in PBS supplemented with 0.1% Tween three times, 10 min each. Membranes were blocked for 1 hour at RT in Pierce Protein Free Blocking Buffer. Nitrocellulose membranes were then incubated with the following antibodies overnight at 4C: mouse monoclonal antibody to GAPDH (1:5000 in Pierce Protein Free Blocking Buffer, 274102; Synaptic Systems); chicken polyclonal antibody to GFP (1:5000 in Pierce Protein Free Blocking Buffer, ab13970; AbCam). Membranes were then washed three times, 10 min each with PBST buffer.

Membranes were then incubated for 1 hour at RT with the following antibodies: goat antibody to chicken AF 488 (1:1000 in Pierce Protein Free Blocking Buffer, ab150169; AbCam); donkey antibody to mouse AF 568 (1:1000 in Pierce Protein Free Blocking Buffer, A 10037; Invitrogen). Membranes were then washed three times, 10 min each with PBST buffer.

Blots were subsequently imaged using blot imager. Quantification of bands was performed using Bio-Rad ImageLab.

### Example 2

### EIF2B5 Protein Gene Replacement in Cells In Vitro

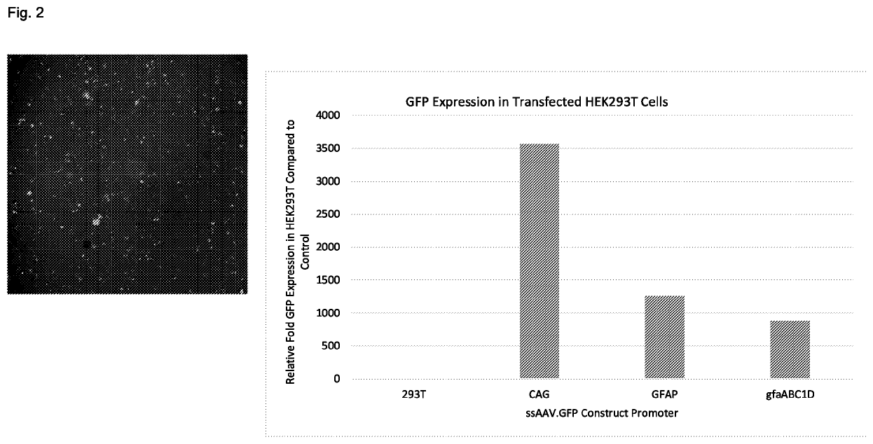

The following experiments were carried out to confirm that AAV9 with the various promoters of interest could be used for the successful ultimate expression of EIF2B5. In this example, however, AAV comprising the promoters of interest with GFP were transfected in HEK cells and in astrocytes derived from fibroblasts in vitro. Expression analyses were completed for the three eGFP constructs: AAV9-CAG-GFP, AAV9-gfaABC(1)D-GFP, and AAV9-GFAP-GFP in HEK cells (FIG. 2) and in astrocytes derived from fibroblasts (FIG. 3).

Specifically, HEK293 cells were transfected by lipofectamine, and 72 hours post-transfection cells were harvested for mRNA expression analysis of eGFP. Although HEK cells were not the ideal cell model (i.e., because astrocytes are the ultimate target cells for these constructs), HEK293 cells were first used to observe expression since they allow for rapid in vitro analysis to determine if the plasmids could successfully express the gene of interest.

At 72 hours post-transfection GFP-positive cells were detectable by microscopy (FIG. 2, left panel). Additionally, quantitative PCR of mRNA expression on cell pellets showed increased expression, CAG˜3500-fold, GFAP˜1200-fold, and GfaABC(1)D˜900-fold over untransfected control cells (FIG. 2, right panel). These results showed successful expression of the transgene driven by the various promoters in HEK cells in vitro. FIG. 3 shows astrocytes derived from fibroblasts stained with GFAP.

### Example 3

### EIF2B5 Gene Replacement in Wild-Type Mice

The following study was carried out to establish that the AAV9-elF2B5 constructs described herein could deliver the transgene, in this example GFP, to cells of interest in the brains of mice, and to compare cell-specific transduction and biodistribution of the various promoters. Healthy C57/BL6 wild-type mice (male and female) were injected at postnatal day 1 (PND1) with the three reporter constructs: AAV9-CAG-GFP, AAV9-gfaABC(1)D-GFP, or AAV9-GFAP-GFP, and sacrificed 28 days post-injection. Tissues of the mice were isolated and analyzed for the presence of GFP.

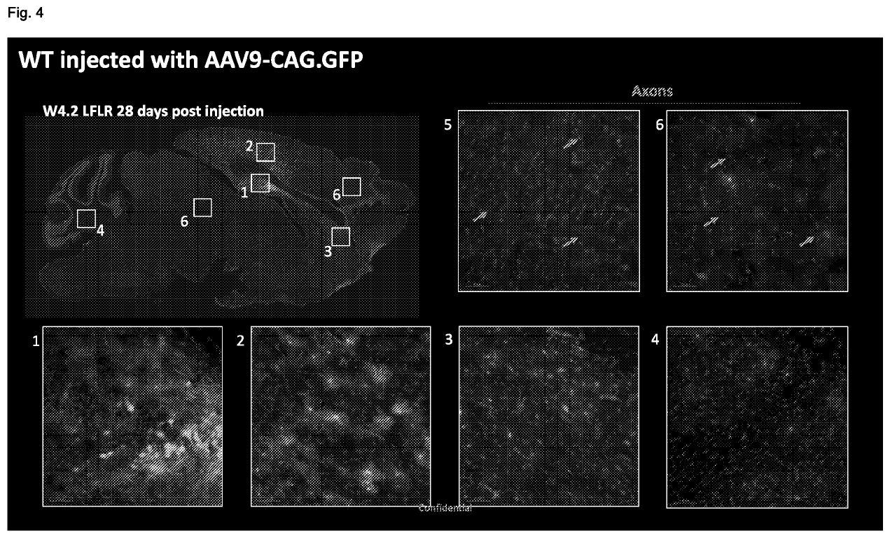

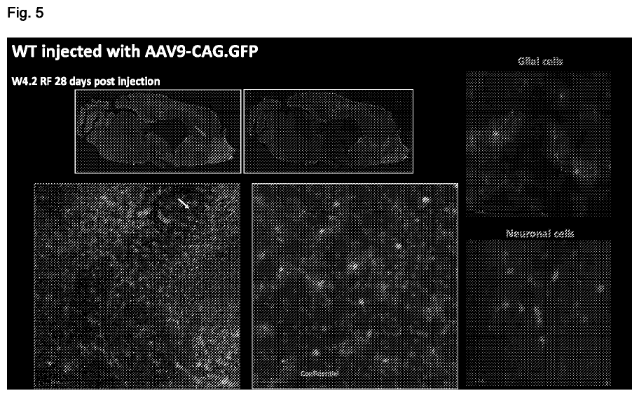

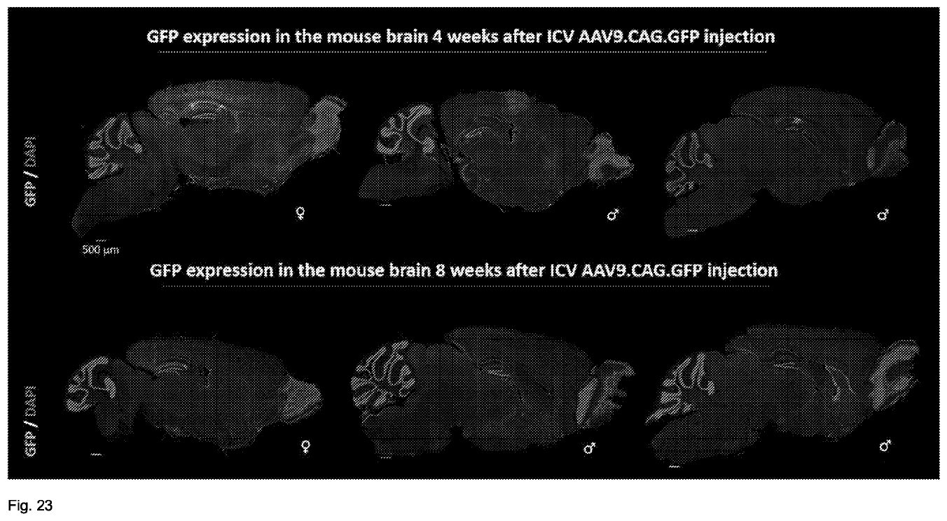

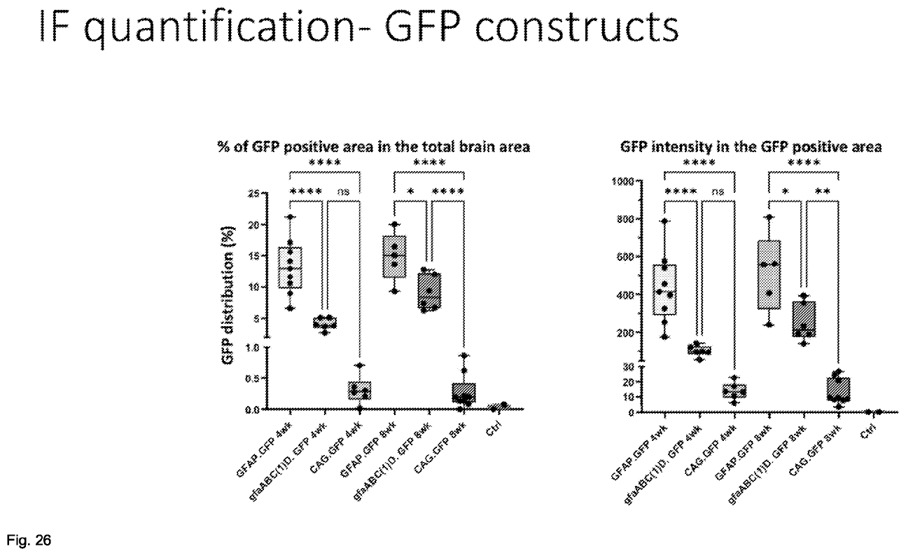

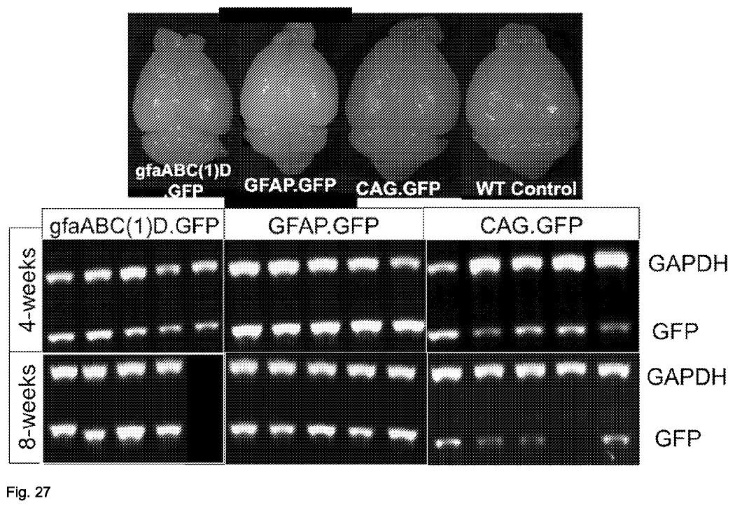

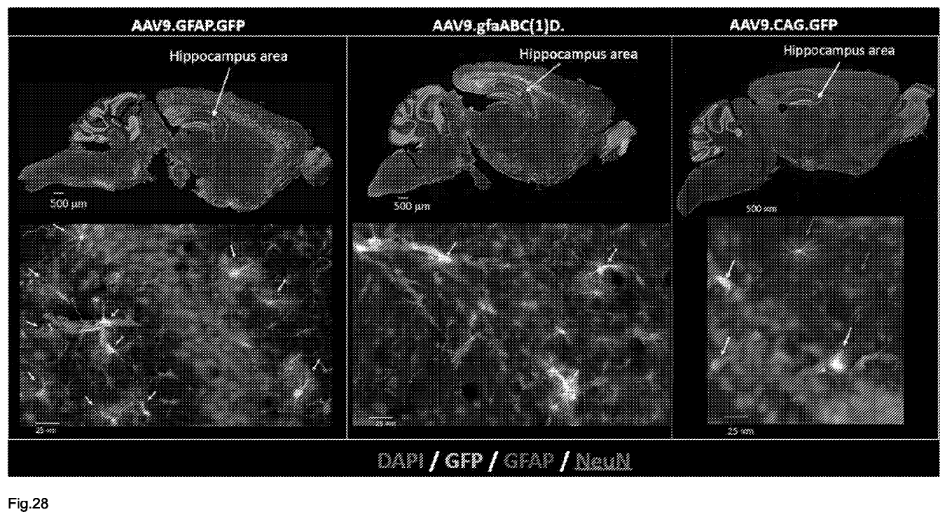

As expected, injection with AAV9-CBA-GFP resulted in a transduction of both glia and neurons (FIGS. 4 and 5). Sagittal sections of the brain were prepared and show distribution of GFP (green), and DAPI (blue) indicates nuclei. Insets 1-6 show higher magnification of the associated voxels on the sagittal section (FIG. 4). Insets show higher magnification of GFP-positive glial and neuronal cells (FIGS. 5 and 28). A survey of multiple mice at both 4 and 8 weeks of age shows distribution throughout the neuroaxis (FIG. 23). The % of positive GFP in the total brain area was quantified and was reduced compared to the GFAP and gfaABC(1)D promoters (FIG. 26). Protein quantification by Western blot is shown in FIG. 27.

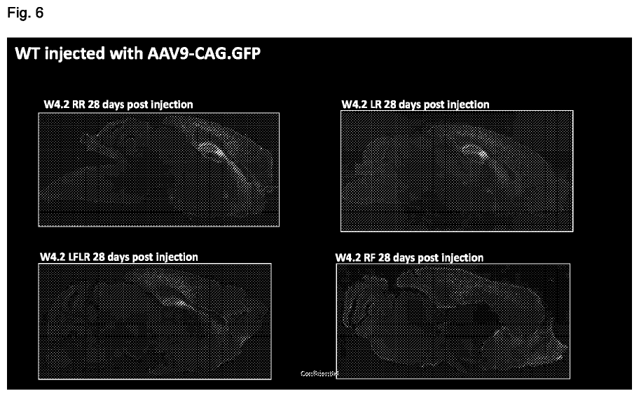

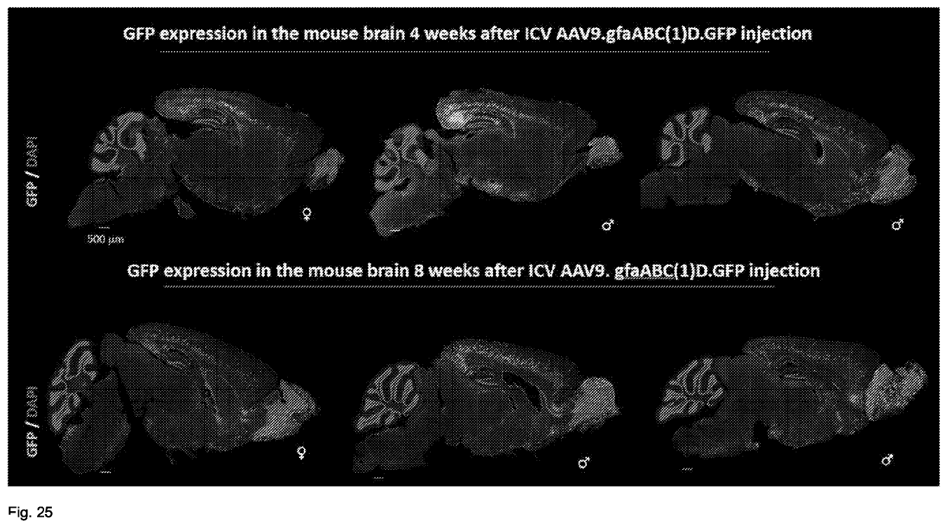

Injection of neonatal wild-type mice with AAV9-gfaABC(1)D-GFP resulted in greater distribution in the white matter and more global distribution throughout the neuroaxis as compared to AAV9-CBA-GFP (FIG. 6). Particularly notable was the increased GFP expression in the caudal brain regions, including the white matter regions of the cerebellum in box 2. Further, expression was primarily in astrocytes, as indicated by the morphology in box 4 and FIG. 28. A survey of multiple mice at both 4 and 8 weeks of age shows intermediate expression throughout the neuroaxis (FIG. 25), which was quantified histologically (FIG. 26), and by Western blot (FIG. 27).

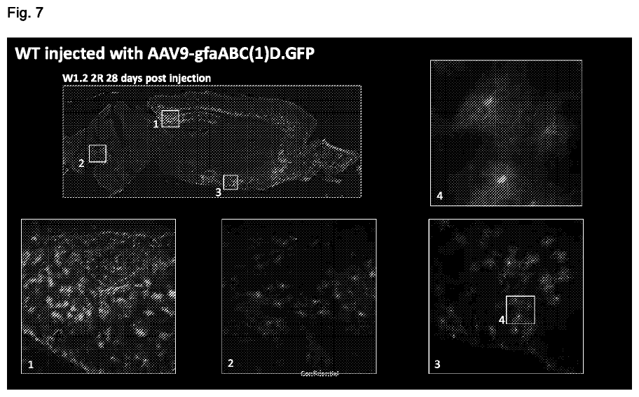

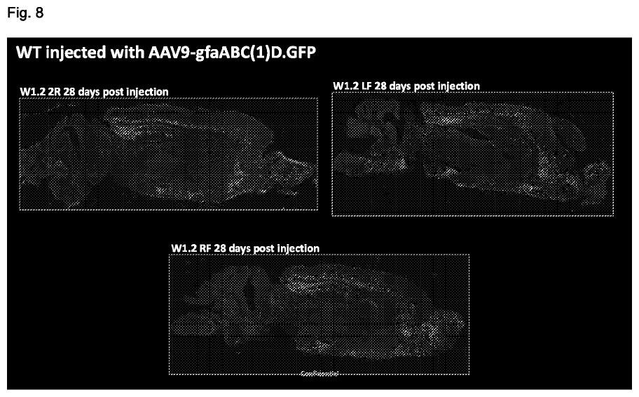

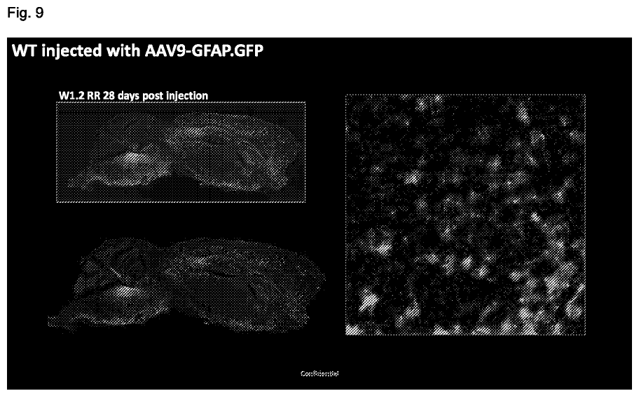

Results of the injection of neonatal wild-type mice with AAV9-gfaABC(1)D-GFP at PND1 and sacrificed 28 days post-injection are shown in FIG. 7. Insets 1-3 show higher magnification of the associated voxels on the sagittal section. Inset 4 shows higher magnification of inset 3 to demonstrate astrocytic morphology. These results show that the use of astrocyte promoters increases distribution in white matter and expression in astrocytes. FIG. 28 provides a comparison of co-labeling between the cohorts.

FIG. 9 shows results from a wild-type mouse injected with AAV9-GFAP-GFP at PND1 and sacrificed 28 days post-injection. The injection of neonatal wild-type mice with AAV9-GFAP-GFP, to date, has resulted in more widespread distribution and greater expression as compared to both AAV9-gfaABC(1)D-GFP and AAV9-CBA-GFP vectors. Expression of the transgene has remained specific to astrocytes.

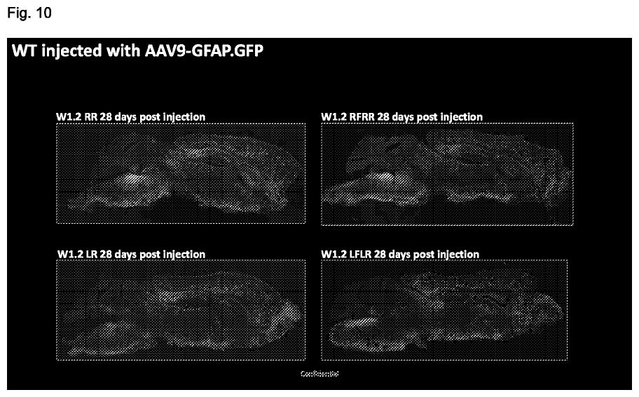

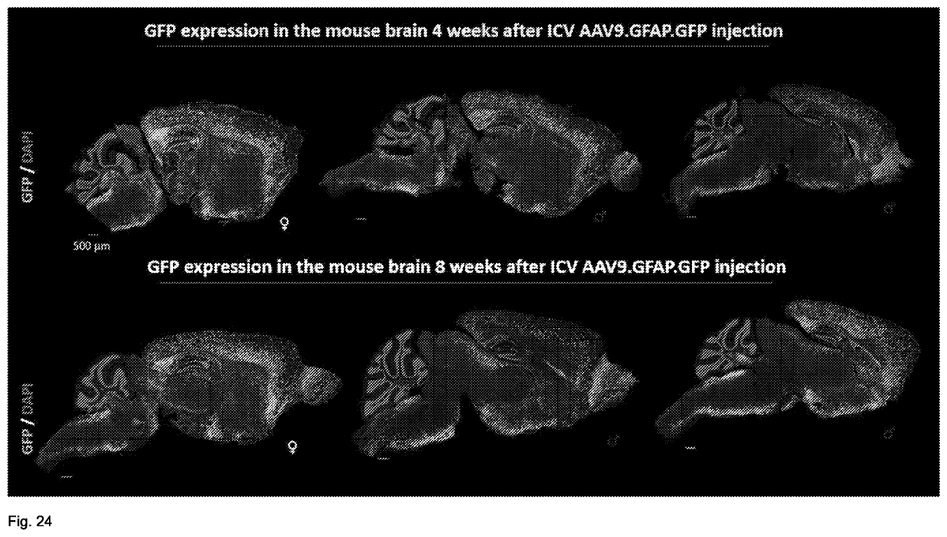

FIG. 10 shows results of experiments with four wild-type mice injected with AAV9-GFAP-GFP at PND1 and sacrificed 28 days post-injection. A sagittal section of the brain shows distribution of GFP (green). Similar biodistribution patterns were seen between the injected mice. A survey of multiple mice at both 4 and 8 weeks of age shows robust expression throughout the neuroaxis (FIG. 24), which was quantified histologically (FIG. 26), and by Western blot (FIG. 27).

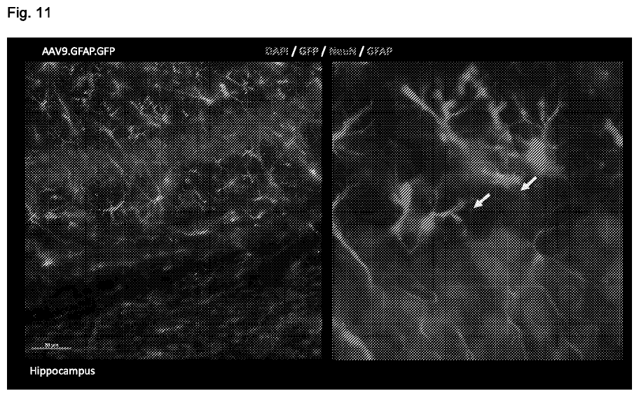

Co-labeling after delivery of AAV9-GFAP-GFP (FIG. 11) shows GFP (green) expression colocalizing with astrocyte marker (GFAP, purple) and not neuronal marker (NeuN, red). GFP expression in astrocytes was confirmed by co-labeling with an antibody specific for astrocytes (GFAP, purple) and neurons (NeuN, red). GFP expression after treatment with AAV9-GFAP-GFP (shown) and AAV9-gfaABC(1)D-GFP (not shown) was clearly present in astrocytes. Thus, the expression of the transgene was confirmed to occur primarily in astrocytes, which are the target cells. These data with AAV driving a reporter protein confirm that use of astrocyte-specific promoters alters the tropism of AAV to greater expression in the white matter and more broad distribution throughout the neuroaxis. FIG. 28 provides a comparison of co-labeling between the cohorts.

### Example 4

### EIF2B5 Gene Replacement in R191H VWM Mice

The following study was carried out to evaluate CSF delivery of AAV9-elF2B5 in a mouse model of elF2B5, i.e., R191H VWM mice, and to determine the effects of the EIF2B5 transgene in this mouse model of VWM. The AAV9-elF2B5 constructs, as described herein, were designed to target astrocytes to also improve oligodendrocyte pathology as it results from a downstream signaling effect secondary to astrocyte dysfunction. In addition, to also evaluate broader CNS transduction, a more ubiquitous promoter also was evaluated.

Pre-symptomatic elF2B5 mice (i.e., R191H VWM mice) were treated by intracerebroventricular (ICV) injection, as described herein above, of one of the following three AAV vector constructs comprising either an astrocyte specific promoter: 1) AAV9-GFAP-elF2B5 or 2) AAV9-gfaABC(1)D-elF2B5 comprising the truncated form of GFAP, or a more ubiquitously expressing promoter: 3) AAV9-CBA-elF2B5. Vectors were delivered in a dose escalating manner. Based on phenotypic characterization of untreated VWM mice, AAV-treated mice were evaluated accordingly. These evaluations included, but were not limited to, weight, motor tests, MRI, and survival. Additional postmortem evaluations include observing and quantifying elF2B5 and select ER stress marker expression levels. Histologically, myelination, ER stress, astrocytes, and oligodendrocytes were assessed. Biodistribution of AAV9 is conducted by qPCR.

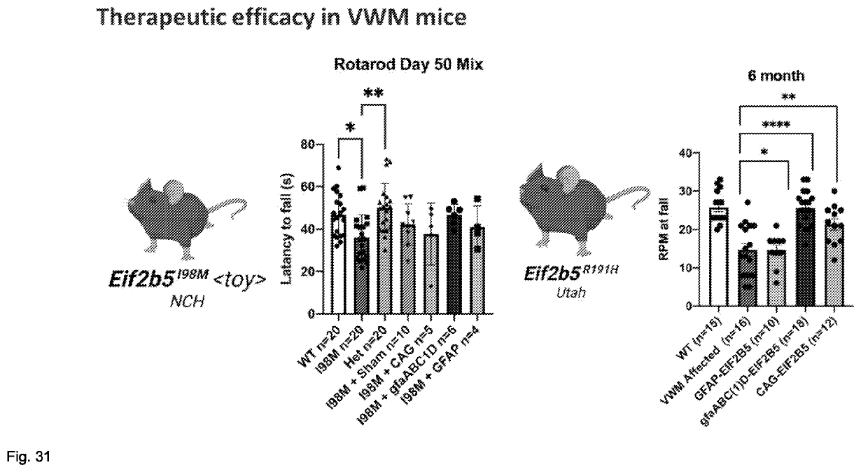

FIG. 31 shows therapeutic efficacy in the treatment of this VWM mouse model as demonstrated by rotarod data at 6 months (FIG. 31, right). The R191H mice at 6 months of age were placed on an accelerating rotating rod (rotarod) and the rotations per minute (RPM) were recorded at which the mice fell off. This assay can accurately measure mobility function which has been shown to be severely affected in this model of VWM, as well as in VWM patients. A higher RPM at fall indicates better mobility. This data shows that the R191H model treated with AAV9.GFAP.elF2B5 displayed a slight, though significant, improvement at 6 months, while the AAV9.CAG.elF2B5 treated mice had better improvement in mobility when compared to the previously stated vector. Finally, mice at 6 months of age treated with AAV9.gfaABC(1)D.elF2B5 had the greatest improvement, and were not significantly different from wild type mice, showing the promise of treatment of this therapeutic vector.

### Example 5

### EIF2B5 Gene Replacement in 198M VWM Mice

The following study was carried out to evaluate CSF delivery of AAV9-elF2B5 in an additional mouse model of elF2B5, i.e., 198M VWM mice, and to determine the effects of the EIF2B5 transgene in this mouse model of VWM. The AAV9-elF2B5 constructs, as described herein, were designed to target astrocytes to also improve oligodendrocyte pathology as it results from a downstream signaling effect secondary to astrocyte dysfunction. In addition, to also evaluate broader CNS transduction, a more ubiquitous promoter also was evaluated.

Pre-symptomatic elF2B5 mice (i.e., 198M VWM mice) were treated by intracerebroventricular (ICV) injection, as described herein above, of one of the following three AAV vector constructs comprising either an astrocyte specific promoter: 1) AAV9-GFAP-elF2B5 or 2) AAV9-gfaABC(1)D-elF2B5 comprising the truncated form of GFAP, or a more ubiquitously expressing promoter: 3) AAV9-CBA-eIF2B5. Vectors were delivered in a dose escalating manner. Based on phenotypic characterization of untreated VWM mice, AAV-treated mice were evaluated accordingly. These evaluations included, but were not limited to, weight, motor tests, MRI, and survival. Additional postmortem evaluations include observing and quantifying elF2B5 and select ER stress marker expression levels. Histologically, myelination, ER stress, astrocytes, and oligodendrocytes were assessed. Biodistribution of AAV9 is conducted by qPCR.

FIG. 31 shows therapeutic efficacy in the treatment of this VWM mouse model as demonstrated by rotarod data at day 50 (left). The 198M mice at 50 days of age were placed on an accelerating rotating rod (rotarod) and the durations at which the mice could stay on (in seconds) were recorded. This assay can accurately measure mobility function which has been shown to be severely affected in this mouse model as well as in VWM patients. In this case, a longer latency to fall indicates better mobility function. This data shows that the untreated 198M mice have a significantly shorter latency to fall than wild type or heterozygous mice. However, 198M mice treated with AAV9.GFAP.elF2B5, AAV9.CAG.elF2B5, and AAV9.gfaABC(1)D.elF2B5 at 50 days of age are statistically indistinguishable from wild type mice, showing promise of these therapeutic vectors.

### Example 6

### Use of a Novel Promoter, Gfa1405, in EIF2B5 Gene Replacement

Glial fibrillary acidic protein (GFAP) is an intermediate-filament protein expressed abundantly and almost exclusively in astrocytes of the CNS. Thus, the GFAP promoter directs astrocyte-specific expression of genes (Brenner et al., J. Neurosci. 1004 March; 14 (3 Pt 1): 1030-7. doi: 10.1523/JNEUROSCI. 14-03-01030.1994). The GFAP promoter, however, is of a rather large size, comprising 2.2 kb. More specifically, due to the packaging size capacity of a single-stranded AAV genome (4.8 kb), GFAP, an astrocytic promoter of 2.2 kb, does not fit well with the EIF2B5 transgene (2.3 kb) along with the regulatory elements needed for appropriate gene expression. Thus, preliminary studies for EIF2B5 expression were carried out that utilized a truncated version of GFAP, i.e., gfaABC(1)D, comprising only 681 bp. The gfaABC(1)D promoter also targets astrocytes, but not at the same potency of the endogenous GFAP promoter.

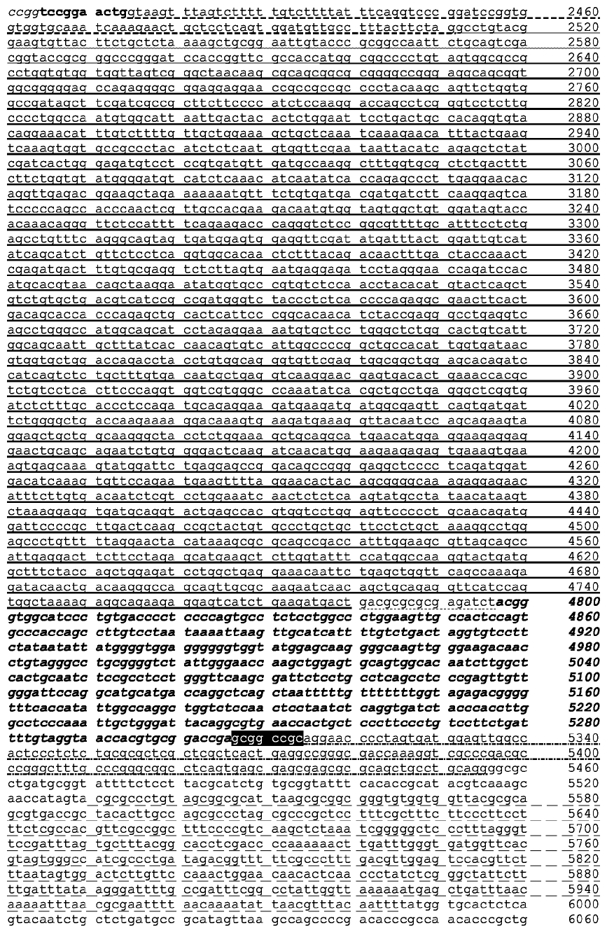

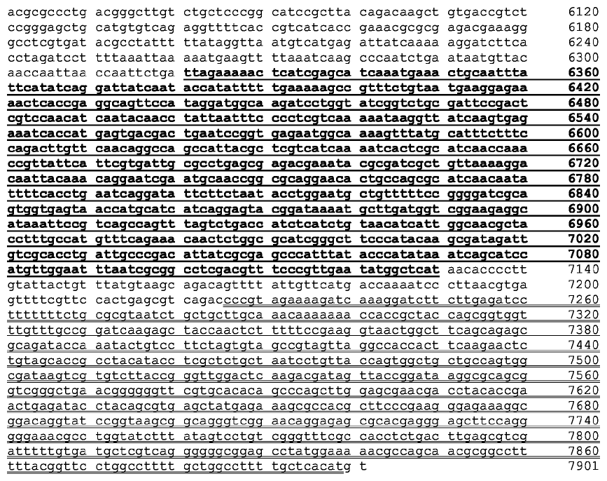

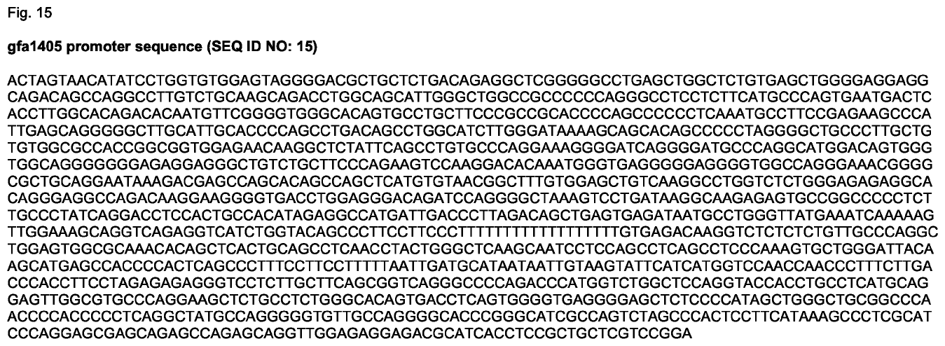

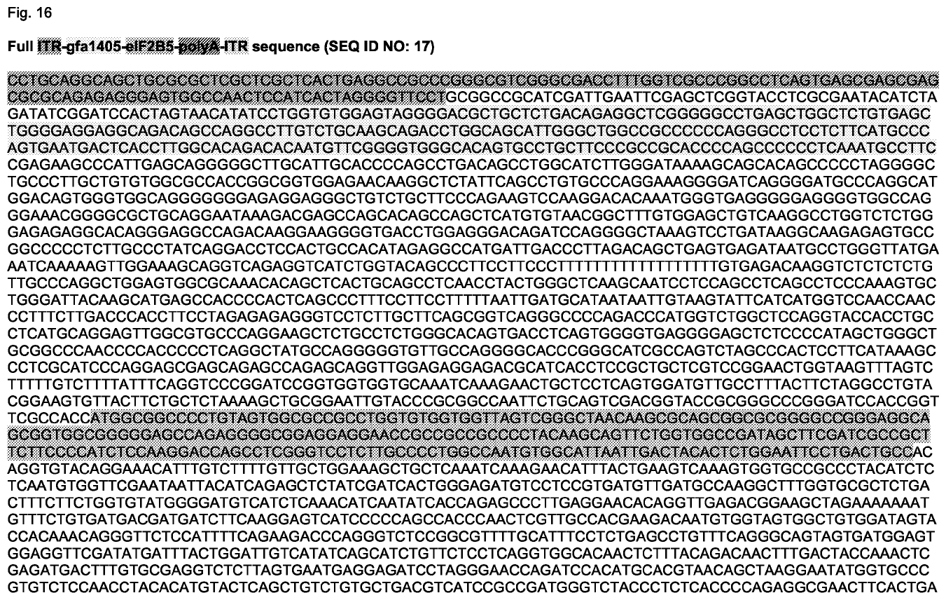

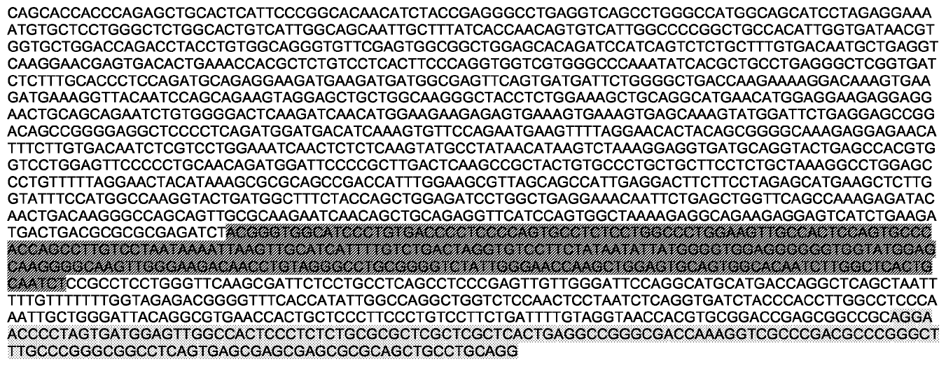

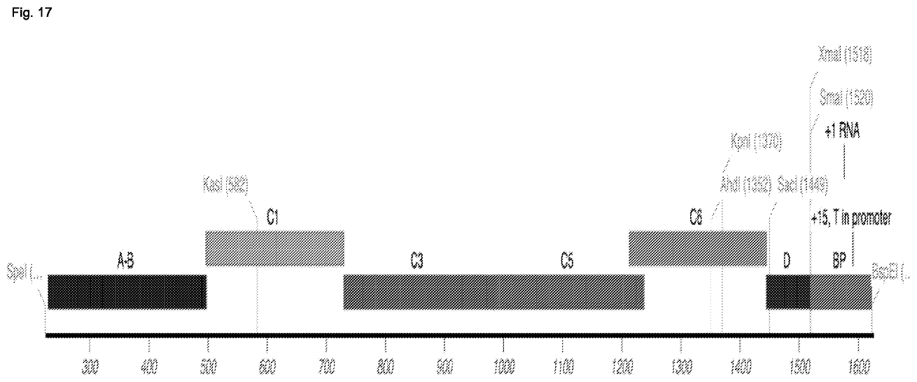

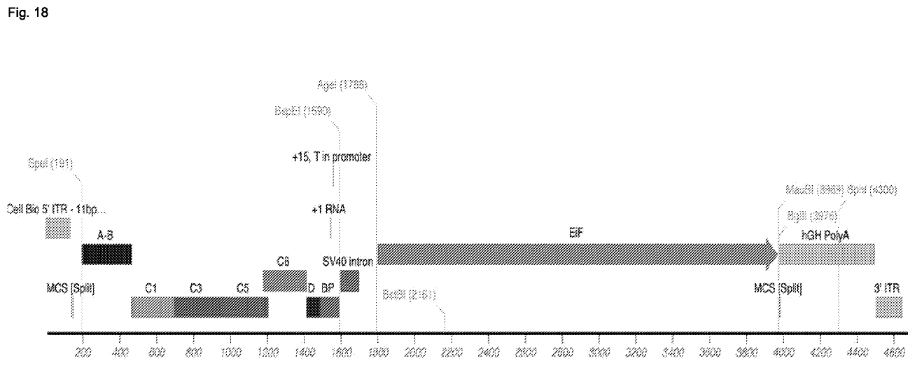

Because of the aforementioned packaging size capacity, a new promoter, the gfa1405 promoter, was designed in order to obtain a smaller astrocyte-specific promoter. The gfa1405 promoter was designed based upon a publication that described the design of the truncated gfaABC(1)D1 promoter (Lee et al., Glia. 2008; 56:481 □493) by systematically returning critical transcription factor binding regions of the C component (C3, C5, and C6), while keeping inhibitory regions (C2) excluded. This novel promoter, gfa1405 promoter, is 1405 base pairs (SEQ ID NO: 15) and when combined with EIF2B5 (i.e., SEQ ID NO: 16 or 17) remains under the packaging threshold for AAV of 4.8 kb (see FIGS. 15 and 16, respectively). Additionally, see the schematic drawings shown in FIGS. 17 and 18 which show the components of the new promoter (FIG. 17) and the gfa1405 promoter with the EIF2B5 transgene (FIG. 18).

TABLE 6
Sequences of the gfa1405 promoter and the gfa1405
promoter with the eIF2B5 transgene sequence.

| Sequence Name | Sequence (5′-3′) | SEQ ID NO: |
|---|---|---|
| gfa1405 promoter (1405 bp) | See FIG. 15. | 15 |
| gfa1405.EIF2B5 with various | See Table 2. | 16 |
| other elements but without 5′ | | |
| and 3′ITRs | | |
| Full ITR-gfa1405-eIF2B5- | See Table 3 and FIG. 16. | 17 |
| polyA-ITR sequence | | |
| (4640 bp) | | |
| AAV9.gfa1405.eiF2B5 | See Table 4 and FIGS. 19 | 18 |
| (7086 bp) | and 21. | |

FIG. 19 provides a schematic drawing of the plasmid, pAAV.gfa1405.elF2B5, which comprises 7086 bp (SEQ ID NO: 18) comprising the gfa1405 promoter with the elF2B5 transgene sequence in the AAV vector. FIG. 21 provides a detailed description of the 7086 bp sequences of SEQ ID NO: 18. Thus, a fourth AAV genome vector construct encoding EIF2B5 was generated.

To test the efficacy of the novel promoter, the gfaABCD1405.eGFP plasmid was transfected into human embryonic kidney cells alongside the ubiquitous CAG.eGFP construct, and the two previously published astrocytic constructs, GFAP.eGFP and gfaABC(1)D.eGFP. Protein extraction and Western blotting methods in detail are described in Example 1.

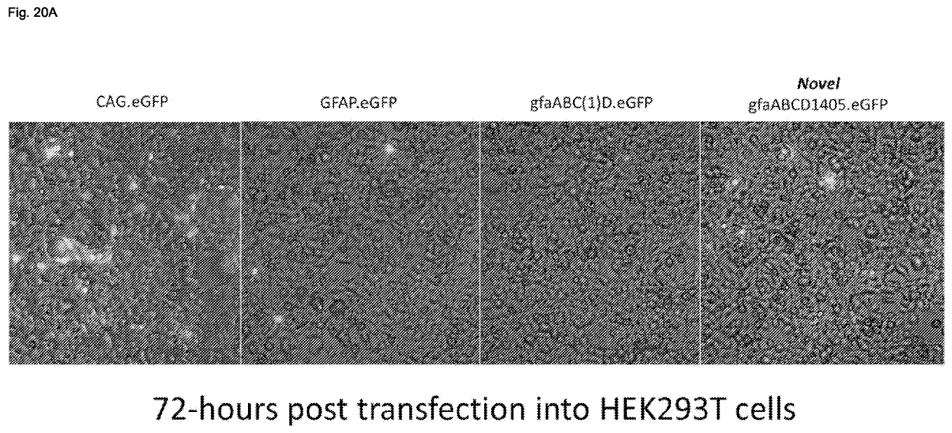

Expression of GFP was observed at 72 hours post-transfection (FIG. 20A) where it was evident that the novel gfaABCD1405 promoter drives expression of GFP in HEK293 cells. After 72 hours post-transfection, cells were harvested and protein was isolated for western blot analysis. Shown in FIG. 20B are results of the quantification of percent CAG expression after a western blot for GFP expression was carried out in triplicate of all constructs in comparison to expression of the house keeping protein, GAPDH. Quantification of the western blot (shown in the bar graph of FIG. 20B) displays that gfaABCD1405 (gfa1405) GFP protein expression was greater than that of the GFAP and gfaABC(1)D constructs, but not as great as that of the ubiquitous promoter CAG construct. This is thought to be because GFAP, gfaABC(1)D and gfaABCD1405 are more specific to astrocytes which would explain lower expression in kidney cells. Nevertheless, results show that the novel promoter, gfa1405, drove a greater amount of protein expression than the other previously published astrocyte-specific promoters.

As a result, the new promoter, gfa1405, was designed to be useful in the treatment of astrocytic and neuronal diseases because it is designed to drive expression of genes in astrocytes while not targeting other cell types. Because astrocytes also cross talk with neurons, targeting astrocytes with this promoter in neuron-specific diseases may also provide therapeutic benefit for neuronal diseases. Thus, diseases or conditions which may benefit from treatments using this promoter to target astrocytes and neurons include, but are not limited to, Vanishing White Matter disease, stroke, migraines, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, and megalencephalic leukoencephalopathy (Ricci et al. J Biol Phys 35, 317-336, doi: 10.1007/s10867-009-9157-9 (2009); Phatnani, H. & Maniatis, T. Astrocytes in neurodegenerative disease. Cold Spring Harb Perspect Biol 7, doi: 10.1101/cshperspect.a020628 (2015); Lanciotti et al., Int J Mol Sci 23, doi: 10.3390/ijms23010274 (2021)).

Consequently, the novel promoter, i.e., gfa1405 (also referred to herein as gfaABCD1405), is useful in a method of treating a subject comprising a mutation in a gene normally expressed in an astrocyte or neuron in a healthy subject, the method comprising administering to the subject an effective amount of a transgene attached to the gfa1405 promoter so that the transgene is expressed in the subject comprising the mutated gene.

### Example 7

Astrocyte-Targeted Gene Therapy with the gfaABCD1405 Promoter

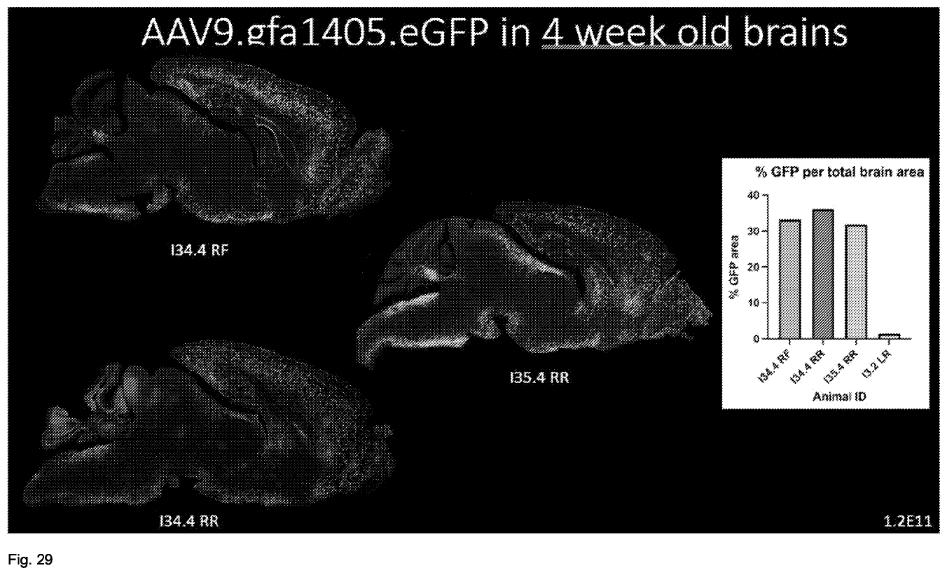

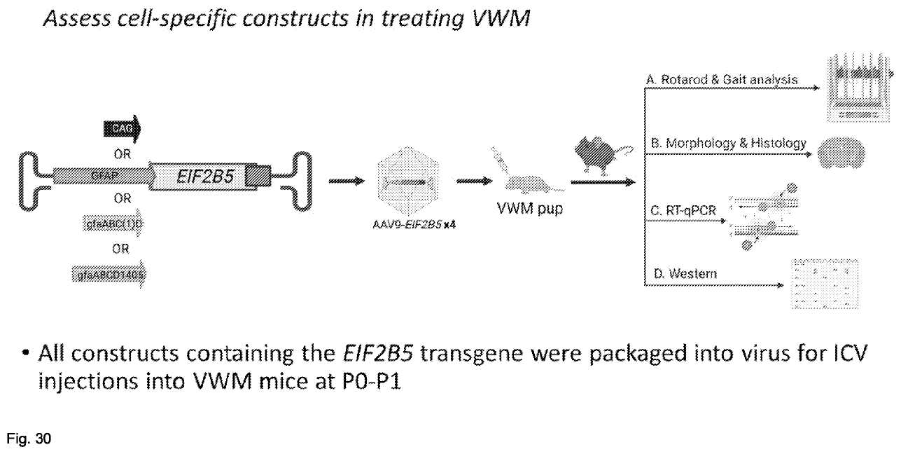

Wild-type (WT) mice were treated PND 0-1 by intracerebroventricular (ICV) delivery with AAV9. gfaABCD1405-GFP at a dose of 1.2E11 vg. Robust GFP expression throughout the neuroaxis was seen 4 weeks after treatment. The expression is comparable to or higher than GFAP expression when measured histologically. Morphology of cells expressing GFP driven by the gfaABCD1405 promoter suggests strong targeting of astrocytes within the CNS, while limiting gene expression in other CNS cell types such as neurons. See FIG. 29. These experiments showed that the astrocyte-specific reporter gfaABCD1405 described herein achieved appropriate transgene expression in astrocytes providing its utility in expressing genes including, but not limited to, eiF2B5.

### Example 8

### Astrocyte-Targeted Gene Therapy Delays Disease Progression, Rescues Body Weight, and Increases Latency to Fall in Models of VWM Disease

Intracerebroventricular (ICV) injections of vectors expressing the reporter protein GFP into wild-type mice revealed that the astrocyte-specific reporter constructs (AAV9.GFAP.eGFP and AAV9.gfaABC(1)D.eGFP) described herein achieved appropriate transgene expression in astrocytes; with AAV9.GFAP.eGFP vector having significantly greater biodistribution throughout the neuroaxis when compared to its truncated, or ubiquitous counterparts (AAV9.gfaABC(1)D.eGFP and AAV9.CAG.eGFP).

To evaluate potential therapeutic efficacy, three constructs driving expression of the EIF2B5 transgene were initially generated. However, due to the size capacity of AAV (4.8 kb), the GFAP promoter (2.2 kb) in combination with the EIF2B5 transgene (2.2 kb) and necessary regulatory elements, led to an oversized construct, subsequent poor packaging, and low viral titers. Because of significant differences in expression between the full-length and truncated astrocyte promoters in the GFP reporter study, studies were designed to increase biodistribution by generating a tailored promoter that restores crucial sequences from the endogenous GFAP promoter into its truncated gfaABC(1)D counterpart. Thus, an intermediate gfaABCD1405 promoter was designed and made, and studies have demonstrated expression comparable to full-length GFAP.

The four AAV constructs were evaluated in two murine VWM models, Eif2b5^{Arg191His} and Eif2b5^{lle98Met}, which display significant gait deficits, myelin loss, and shortened life span. Disease progression was monitored. AAV9-gfaABC(1)D-EIF2B5 treated Eif2b5^{Arg191His} and Eif2b5^{lle98Met} mice to have reached >50 days of age are indistinguishable from normal mice on rotarod and significantly (P≤0.001) improved as compared to untreated R191H affected mice (FIG. 31). Treatment with vectors including the CAG and GFAP promoters are demonstrating an intermediate effect in the mice. Eif2b5^{Arg191His} and Eif2b5^{lle98Met} treated with AAV9-gfaABCD1405-EIF2B5 have not yet reached 50 days of age. Data to date indicate that the astrocyte-targeted gene therapy described herein, particularly AAV9-gfaABC(1)D-EIF2B5, is able to significantly increase latency to fall on rotarod in both disease models to a significantly greater extent than ubiquitous (AAV9-CAG-EIF2B5) gene therapy (see FIG. 31).

The improved latency to fall on rotarod caused by these therapies indicates increased coordination and motor function, which has been shown to be heavily limited in both mouse models as well as in VWM patients. RPM at fall specifically shows how fast the rotating rod is accelerating; therefore, higher RPM measurements are also indicative of increased coordination and motor function. The difference between these two measurements is that latency to fall is measuring duration (i.e., time on the rotarod) while RPM is measuring how fast the rotarod is rotating. These two variables are linked; therefore, longer duration (more time) means greater RPMs and vice versa. Significant improvements in both measurements after treatment indicates the promise of the disclosed methods of therapy as a potential therapy for VWM.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word □comprise □and variations such as □comprises □and □comprising will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control. References referred to herein with numbering are provided with the full citation as shown herein below.

### REFERENCES

1. Fogli A, Schiffmann R, Bertini E, Ughetto S, Combes P, Eymard-Pierre E, et al. The effect of genotype on the natural history of elF2B-related leukodystrophies. Neurology. 2004; 62 (9): 1509-17.
2. van der Knaap M S, Pronk J C, and Scheper G C. Vanishing white matter disease. Lancet Neurol. 2006; 5 (5): 413-23.
3. Bonkowsky J L, Nelson C, Kingston J L, Filloux F M, Mundorff M B, and Srivastava R. The burden of inherited leukodystrophies in children. Neurology. 2010; 75 (8): 718-25.
4. Zhou L, Zhang H H, Chen N, Zhang Z B, Liu M, Dai L F, et al. [Clinical features of 54 cases of leukoencephalopathy with vanishing white matter disease in children]. Zhonghua Er Ke Za Zhi. 2019; 57 (11): 837-43.
5. Dietrich J, Lacagnina M, Gass D, Richfield E, Mayer-Proschel M, Noble M, et al. EIF2B5 mutations compromise GFAP+ astrocyte generation in vanishing white matter leukodystrophy. Nat Med. 2005; 11 (3): 277-83.
6. Geva M, Cabilly Y, Assaf Y, Mindroul N, Marom L, Raini G, et al. A mouse model for eukaryotic translation initiation factor 2B-leucodystrophy reveals abnormal development of brain white matter. Brain. 2010; 133 (Pt 8): 2448-61.
7. Dooves S, Bugiani M, Postma N L, Polder E, Land N, Horan S T, et al. Astrocytes are central in the pathomechanisms of vanishing white matter. J Clin Invest. 2016; 126 (4): 1512-24.
8. Terumitsu-Tsujita M, Kitaura H, Miura I, Kiyama Y, Goto F, Muraki Y, et al. Glial pathology in a novel spontaneous mutant mouse of the Eif2b5 gene: a vanishing white matter disease model. J Neurochem. 2019.
9. Cearley C N, and Wolfe J H. Transduction characteristics of adeno-associated virus vectors expressing cap serotypes 7, 8, 9, and Rh10 in the mouse brain. Mol Ther. 2006; 13 (3): 528-37.
10. Swain G P, Prociuk M, Bagel J H, O Donnell P, Berger K, Drobatz K, et al. Adeno-associated virus serotypes 9 and rh10 mediate strong neuronal transduction of the dog brain. Gene Ther. 2014; 21 (1): 28-36.
11. Samaranch L, Salegio E A, San Sebastian W, Kells A P, Foust K D, Bringas J R, et al. Adeno-associated virus serotype 9 transduction in the central nervous system of nonhuman primates. Human gene therapy. 2012; 23 (4): 382-9.
12. Hordeaux J, Hinderer C, Buza E L, Louboutin J P, Jahan T, Bell P, et al. Safe and Sustained Expression of Human Iduronidase After Intrathecal Administration of Adeno-Associated Virus Serotype 9 in Infant Rhesus Monkeys. Hum Gene Ther. 2019; 30 (8): 957-66.
13. von Jonquieres G, Mersmann N, Klugmann C B, Harasta A E, Lutz B, Teahan O, et al. Glial promoter selectivity following AAV-delivery to the immature brain. PLoS One. 2013; 8 (6): e65646.
14. Lee, Y., Messing, A., Su, M. & Brenner, M. GFAP promoter elements required for region-specific and astrocyte-specific expression. Glia 56, 481-493, doi: 10.1002/glia.20622 (2008).
15. Ricci, G., Volpi, L., Pasquali, L., Petrozzi, L. & Siciliano, G. Astrocyte-neuron interactions in neurological disorders. J Biol Phys 35, 317-336, doi: 10.1007/s10867-009-9157-9 (2009).
16. Phatnani, H. & Maniatis, T. Astrocytes in neurodegenerative disease. Cold Spring Harb Perspect Biol 7, doi: 10.1101/cshperspect.a020628 (2015).
17. Lanciotti, A., Brignone, M. S., Macioce, P., Visentin, S. & Ambrosini, E. Human iPSC-Derived Astrocytes: A Powerful Tool to Study Primary Astrocyte Dysfunction in the Pathogenesis of Rare Leukodystrophies. Int J Mol Sci 23, doi: 10.3390/ijms23010274 (2021).

We claim:

1. A nucleic acid comprising a polynucleotide comprising
(a) one or more regulatory control element(s); and
(b) a Eukaryotic Translation Initiation Factor 2B Subunit Epsilon 5 (EIF2B5) cDNA sequence.

2. The nucleic acid of claim 1, wherein the EIF2BB5 cDNA comprises
(a) a nucleotide sequence having at least 80% sequence identity to SEQ ID NO: 1;
(b) the nucleotide sequence set forth in SEQ ID NO: 1; or
(c) a nucleotide sequence encoding EIF2BB5 comprising the amino acid sequence set forth in SEQ ID NO: 2.

3. The nucleic acid of any one of claims 1-3, wherein the one or more regulatory control element(s) is a CAG promoter, a gfaABC1D promoter, a GFAP promoter, or a gfa1405 promoter.

4. The nucleic acid of any one of claims 1-4, wherein the regulatory control element comprises
(a) a nucleotide sequence having at least 80% sequence identity to SEQ ID NO: 3, 4, 5, or 15; or
(b) the nucleotide sequence set forth in SEQ ID NO: 3, 4, 5, or 15.

5. The nucleic acid of any one of claims 1-4 further comprising an SV40 intron and a post-transcriptional polyadenylation (polyA) sequence.

6. The nucleic acid of claim 5 comprising
(a) a nucleotide sequence having at least 80% sequence identity to any one of SEQ ID NOs: 6-8 and 16; or
(b) the nucleotide sequence set forth in any one of SEQ ID NOs: 6-8 and 16.

7. The nucleic acid of any one of claims 1-6 further comprising an inverted terminal repeat sequence.

8. The nucleic acid of claim 7 comprising
(a) a nucleotide sequence having at least 80% sequence identity to any one of SEQ ID NOs: 9-11 and 17; or
(b) the nucleotide sequence set forth in any one of SEQ ID NOs: 9-11 and 17.

9. The nucleic acid of any one of claims 1-8 comprising
(a) a nucleotide sequence having at least 80% sequence identity to any one of SEQ ID NOs: 12-14 and 18; or
(b) the nucleotide sequence set forth in any one of SEQ ID NOs: 12-14 and 18.

10. A nanoparticle, extracellular vesicle, exosome, or vector comprising the nucleic acid of any one of claims 1-8 or a combination of any one or more thereof.

11. The vector of claim 10, wherein the vector is a viral vector.

12. The viral vector of claim 11, wherein the viral vector is an adeno-associated virus (AAV), adenovirus, lentivirus, retrovirus, poxvirus, baculovirus, herpes simplex virus, vaccinia virus, or a synthetic virus.

13. The viral vector of claim 11 or 12, wherein the viral vector is an AAV.

14. The viral vector of claim 13, wherein the AAV lacks rep and cap genes.

15. The viral vector of claim 13 or 14, wherein the AAV is a recombinant AAV (rAAV), a self-complementary recombinant AAV (scAAV), or a single-stranded recombinant AAV (ssAAV).

16. The AAV of any one of claims 13-15, wherein the AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh. 10, MyoAAV 1A, AAVMYO, or AAV-B1, AAV2/1, AAV2/8, AAV2/9, or any of their derivatives.

17. The AAV of any one of claims 13-16, wherein the AAV is AAV9.

18. An rAAV particle comprising the AAV of any one of claims 12-16.

19. A composition comprising:
(a) the nucleic acid of any one of claims 1-9;
(b) the nanoparticle, extracellular vesicle, exosome, or vector of claim 10;
(c) the viral vector of any one of claims 11-17; or
(d) the rAAV particle of claim 18; and
a pharmaceutically acceptable carrier.

20. The composition of claim 19, wherein the composition is formulated for intrathecal intracerebroventricular, intracerebral, intravenous, intracisternal, or aerosol delivery.

21. A method of increasing the expression of a EIF2B5 gene or EIF2B5 protein in a cell comprising contacting the cell with
(a) the nucleic acid of any one of claims 1-9;
(b) the nanoparticle, extracellular vesicle, exosome, or vector of claim 10;
(c) the viral vector of any one of claims 11-17;
(d) the rAAV particle of claim 18; or
(e) the composition of claim 19 or 20.

22. The method of claim 21, wherein the cell is an astrocyte.

23. The method of claim 20 or 21, wherein the cell is a human cell.

24. The method of claim 22, wherein the cell is in a human subject.

25. A method of treating a subject comprising a EIF2BB5 gene mutation comprising administering to the subject an effective amount of
(a) the nucleic acid of any one of claims 1-9;
(b) the nanoparticle, extracellular vesicle, exosome, or vector of claim 10;
(c) the viral vector of any one of claims 11-17;
(d) the rAAV particle of claim 18; or
(e) the composition of claim 19 or 20.

26. The method of claim 24, wherein the subject is a human subject.

27. The method of claim 25 or 26, wherein the EIF2BB5 gene mutation causes a subject to suffer from or be at risk of suffering from a leukoencephalopathy, a megalencephalic leukoencephalopathy, a leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, and/or giant axonal neuropathy.

28. The method of claim 27, wherein the leukoencephalopathy or leukodystrophy is Vanishing White Matter Disease (VWM).

29. The method of any one of claims 25-28, further comprising administering any one or more of a corticosteroid, rituximab, and rapamycin to the subject.

30. The method of any one of claims 25-29, wherein the nucleic acid, nanoparticle, extracellular vesicle, exosome, vector, rAAV particle, or composition is administered by intrathecal intracerebroventricular, intracerebral, intravenous, intracisternal, or aerosol delivery.

31. Use of
(a) the nucleic acid of any one of claims 1-9;
(b) the nanoparticle, extracellular vesicle, exosome, or vector of claim 10;
(c) the viral vector of any one of claims 11-17;
(d) the rAAV particle of claim 18; or
(e) the composition of claim 19 or 20
for the preparation of a medicament for increasing expression of the EIF2B5 gene or protein in a cell.

32. The use of claim 31, wherein the cell is in a human subject.

33. Use of
(a) the nucleic acid of any one of claims 1-9;
(b) the nanoparticle, extracellular vesicle, exosome, or vector of claim 10;
(c) the viral vector of any one of claims 11-17;
(d) the rAAV particle of claim 18; or
(e) the composition of claim 19 or 20
in treating a subject comprising a mutant EIF2B5 gene.

34. The use of claim 33, wherein the subject is a human subject.

35. The use of any one of claims 31-34, wherein the subject suffers from a EIF2B5 mutation.

36. The use of claim 35, wherein the EIF2B5 mutation is associated with a leukoencephalopathy, a megalencephalic leukoencephalopathy, a leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, and/or vanishing white matter (VWM) disease.

37. The use of any one of claims 31-36, wherein the medicament is administered with any one or more of a corticosteroid, rituximab, and rapamycin.

38. The use of any one of claims 31-37, wherein the medicament is formulated for intrathecal intracerebroventricular, intracerebral, intravenous, intracisternal, or aerosol delivery.

39. A composition for treating a EIF2B5 gene mutation, a leukoencephalopathy, leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, and/or a megalencephalic leukoencephalopathy in a subject, wherein the composition comprises
(a) the nucleic acid of any one of claims 1-9;
(b) the nanoparticle, extracellular vesicle, exosome, or vector of claim 10;
(c) the viral vector of any one of claims 11-17;
(d) the rAAV particle of claim 18; or
(e) the composition of claim 19 or 20.

40. The composition of claim 39, wherein the subject is a human subject.

41. The composition of claim 39 or 40, wherein the leukoencephalopathy or leukodystrophy is Vanishing White Matter Disease (VWM).

42. The
(a) nucleic acid of any one of claims 1-9;
(b) nanoparticle, extracellular vesicle, exosome, or vector of claim 10;
(c) viral vector of any one of claims 11-17;
(d) rAAV particle of claim 18;
(e) composition of claim 19 or 20;
(f) method of any one of claims 21-30; or
(g) use of any one of claims 31-38,
wherein the nucleic acid, nanoparticle, extracellular vesicle, exosome, vector, viral vector, composition, or medicament is formulated for intrathecal injection into the cerebrospinal fluid (CSF), intravenous injection into the blood stream, intracerebral injection, intracerebroventricular injection, intracisternal injection, or for aerosol administration.

43. A nucleic acid comprising a gfa1405 promoter comprising
(a) a nucleotide sequence having at least 80% sequence identity to SEQ ID NO: 15; or
(b) the nucleotide sequence set forth in SEQ ID NO: 15.

44. The nucleic acid of claim 43 further comprising an inverted terminal repeat sequence.

45. The nucleic acid of claim 44 comprising
(a) a nucleotide sequence having at least 80% sequence identity to SEQ ID NO: 17; or
(b) the nucleotide sequence set forth in SEQ ID NO: 17.

46. The nucleic acid of any one of claims 43-45 comprising
(a) a nucleotide sequence having at least 80% sequence identity to SEQ ID NO: 18; or
(b) the nucleotide sequence set forth in SEQ ID NO: 18.

47. A nanoparticle, extracellular vesicle, exosome, or vector comprising the nucleic acid of any one of claims 43-45 or a combination of any one or more thereof.

48. The vector of claim 47, wherein the vector is a viral vector.

49. The viral vector of claim 48, wherein the viral vector is an adeno-associated virus (AAV), adenovirus, lentivirus, retrovirus, poxvirus, baculovirus, herpes simplex virus, vaccinia virus, or a synthetic virus.

50. The viral vector of claim 48 or 49, wherein the viral vector is an AAV.

51. The viral vector of claim 50, wherein the AAV lacks rep and cap genes.

52. The viral vector of claim 50 or 51, wherein the AAV is a recombinant AAV (rAAV), a self-complementary recombinant AAV (scAAV), or a single-stranded recombinant AAV (ssAAV).

53. The AAV of any one of claims 50-52, wherein the AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh. 10, MyoAAV 1A, AAVMYO, or AAV-B1, AAV2/1, AAV2/8, AAV2/9, or any of their derivatives.

54. The AAV of any one of claims 50-53, wherein the AAV is AAV9.

55. An rAAV particle comprising the AAV of any one of claims 50-54.

56. A composition comprising:
(a) the nucleic acid of any one of claims 43-46;
(b) the nanoparticle, extracellular vesicle, exosome, or vector of claim 47;
(c) the viral vector of any one of claims 48-52;
(d) the AAV of claim 53 or 54; or
(e) the rAAV particle of claim 55; and
a pharmaceutically acceptable carrier.

57. The composition of claim 56, wherein the composition is formulated for intrathecal intracerebroventricular, intracerebral, intravenous, intracisternal, or aerosol delivery.

58. A method of increasing the expression of a gene or a protein in a cell comprising contacting the cell with
(a) the nucleic acid of any one of claims 43-46;
(b) the nanoparticle, extracellular vesicle, exosome, or vector of claim 47;
(c) the viral vector of any one of claims 48-52;
(d) the AAV of claim 53 or 54;
(e) the rAAV particle of claim 55; or
(f) the composition of claim 56 or 57.

59. The method of claim 58, wherein the cell is an astrocyte or a neuron.

60. The method of claim 58 or 59, wherein the cell is a human cell.

61. The method of claim 60, wherein the cell is in a human subject.

62. A method of treating a subject comprising a mutation in a gene normally expressed in an astrocyte or neuron of the subject, the method comprising administering to the subject an effective amount of
(a) the nucleic acid of any one of claims 43-46;
(b) the nanoparticle, extracellular vesicle, exosome, or vector of claim 47;
(c) the viral vector of any one of claims 48-52;
(d) the AAV of claim 53 or 54;
(e) the rAAV particle of claim 55; or
(f) the composition of claim 56 or 57.

63. The method of claim 62, wherein the subject is a human subject.

64. The method of claim 62 or 63, wherein the gene mutation causes a subject to suffer from or be at risk of suffering from an astrocyte or neuronal disorder or disease.

65. The method of any one of claims 62-64, wherein the disorder or disease is a leukoencephalopathy or leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, and/or a megalencephalic leukoencephalopathy.

66. The method of claim 65, wherein the leukoencephalopathy or leukodystrophy is Vanishing White Matter Disease (VWM).

67. The method of any one of claims 62-66, further comprising administering any one or more of a corticosteroid, rituximab, and rapamycin to the subject.

68. The method of any one of claims 62-67, wherein the nucleic acid, nanoparticle, extracellular vesicle, exosome, vector, rAAV particle, or composition is administered by intrathecal intracerebroventricular, intracerebral, intravenous, intracisternal, or aerosol delivery.

69. Use of
(a) the nucleic acid of any one of claims 43-46;
(b) the nanoparticle, extracellular vesicle, exosome, or vector of claim 47;
(c) the viral vector of any one of claims 48-52;
(d) the AAV of claim 53 or 54;
(e) the rAAV particle of claim 55; or
(f) the composition of claim 56 or 57 for the preparation of a medicament for increasing expression of a gene or protein in a cell.

70. The use of claim 69, wherein the cell is in a human subject.

71. Use of
(a) the nucleic acid of any one of claims 43-46;
(b) the nanoparticle, extracellular vesicle, exosome, or vector of claim 47;
(c) the viral vector of any one of claims 48-52;
(d) the AAV of claim 53 or 54;
(e) the rAAV particle of claim 55;
(f) the composition of claim 56 or 57; or
(g) the method of any one of claims 58-68 in treating a subject comprising a mutant gene.

72. The use of claim 71, wherein the subject is a human subject.

73. The use of any one of claims 69-72, wherein the subject suffers from a gene mutation or a disorder or disease affecting the central nervous system and/or brain.

74. The use of any one of claims 69-72, wherein the subject suffers from a gene mutation or a disorder or disease affecting astrocytes or neurons of the brain.

75. The use of claim 73 or 74, wherein the subject suffers from a leukoencephalopathy or leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, and/or a megalencephalic leukoencephalopathy.

76. The use of claim 75, wherein the leukoencephalopathy or leukodystrophy is Vanishing White Matter Disease (VWM).

77. The use of any one of claims 69-76, wherein the medicament is administered with any one or more of a corticosteroid, rituximab, and rapamycin.

78. The use of any one of claims 69-77, wherein the medicament is formulated for intrathecal intracerebroventricular, intracerebral, intravenous, intracisternal, or aerosol delivery.

79. A composition for treating a gene mutation, or a disease or disorder in the central nervous system and/or brain of a subject, wherein the composition comprises
(a) the nucleic acid of any one of claims 43-46;
(b) the nanoparticle, extracellular vesicle, exosome, or vector of claim 47;
(c) the viral vector of any one of claims 48-52;
(d) the AAV of claim 53 or 54;
(e) the rAAV particle of claim 55; or
(f) the composition of claim 56 or 57.

80. The composition of claim 79, wherein the subject is a human subject.

81. The composition of claim 79 or 80, wherein the subject suffers from a gene mutation or a disorder or disease affecting astrocytes or neurons of the brain.

82. The composition of 80 or 81, wherein the subject suffers from a leukoencephalopathy or leukodystrophy, a stroke, a migraine, epilepsy, multiple sclerosis (MS), Parkinson's disease (PD), Alzheimer's disease (AD), astrogliosis in aging, Huntington's Disease (HD), amyotrophic lateral sclerosis (ALS), Alexander disease, hepatic encephalopathy (HE), Aicardi□Goutières syndrome, CLC-2-related disease, oculodentodigital dysplasia, giant axonal neuropathy, and/or a megalencephalic leukoencephalopathy.

83. The composition of claim 82, wherein the leukoencephalopathy or leukodystrophy is Vanishing White Matter Disease (VWM).

84. The
(a) nucleic acid of any one of claims 43-46;
(b) nanoparticle, extracellular vesicle, exosome, or vector of claim 47;
(c) viral vector of any one of claims 48-52;
(d) AAV of claim 53 or 54;
(e) rAAV particle of claim 55; or
(f) composition of claim 56 or 57, or 79-83;
(g) method of any one of claims 58-68; or
(h) use of any one of claims 69-78,
wherein the nucleic acid, nanoparticle, extracellular vesicle, exosome, vector, viral vector, composition, or medicament is formulated for intrathecal injection into the cerebrospinal fluid (CSF), intravenous injection into the blood stream, intracerebral injection, intracerebroventricular injection, intracisternal injection, or for aerosol administration.


SEQUENCE LISTING (18 sequences)

SEQ ID NO: 1 - DNA, 2166 bp, synthetic construct
  source 1..2166: mol_type = other DNA
atggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactga

SEQ ID NO: 2 - protein, 721 aa, synthetic construct
MAAPVVAPPGVVVSRANKRSGAGPGGSGGGGARGAEEEPPPPLQAVLVADSFDRRFFPISKDQPRVLLPLANVALIDYTLEFLTATGVQETFVFCCWKAAQIKEHLLKSKWCRPTSLNVVRIITSELYRSLGDVLRDVDAKALVRSDFLLVYGDVISNINITRALEEHRLRRKLEKNVSVMTMIFKESSPSHPTRCHEDNVVVAVDSTTNRVLHFQKTQGLRRFAFPLSLFQGSSDGVEVRYDLLDCHISICSPQVAQLFTDNFDYQTRDDFVRGLLVNEEILGNQIHMHVTAKEYGARVSNLHMYSAVCADVIRRWVYPLTPEANFTDSTTQSCTHSRHNIYRGPEVSLGHGSILEENVLLGSGTVIGSNCFITNSVIGPGCHIGDNVVLDQTYLWQGVRVAAGAQIHQSLLCDNAEVKERVTLKPRSVLTSQVVVGPNITLPEGSVISLHPPDAEEDEDDGEFSDDSGADQEKDKVKMKGYNPAEVGAAGKGYLWKAAGMNMEEEEELQQNLWGLKINMEEESESESEQSMDSEEPDSRGGSPQMDDIKVFQNEVLGTLQRGKEENISCDNLVLEINSLKYAYNISLKEVMQVLSHVVLEFPLQQMDSPLDSSRYCALLLPLLKAWSPVFRNYIKRAADHLEALAAIEDFFLEHEALGISMAKVLMAFYQLEILAEETILSWFSQRDTTDKGQQLRKNQQLQRFIQWLKEAEEESSEDD

SEQ ID NO: 3 - DNA, 581 bp, synthetic construct
  source 1..581: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggc

SEQ ID NO: 4 - DNA, 647 bp, synthetic construct
  source 1..647: mol_type = other DNA
ggtaccaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagcca

SEQ ID NO: 5 - DNA, 2208 bp, synthetic construct
  source 1..2208: mol_type = other DNA
cccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccgg

SEQ ID NO: 6 - DNA, 3484 bp, synthetic construct
  source 1..3484: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 7 - DNA, 3549 bp, synthetic construct
  source 1..3549: mol_type = other DNA
ggtaccaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccatccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 8 - DNA, 5110 bp, synthetic construct
  source 1..5110: mol_type = other DNA
cccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccggtccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

SEQ ID NO: 9 - DNA, 3829 bp, synthetic construct
  source 1..3829: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcgagctcggtacctcgcgaatacatctagatatcggatccactagtcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 10 - DNA, 3894 bp, synthetic construct
  source 1..3894: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcgagctcggtacctcgcgaatacatctagatatcggatccactagtggtaccaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccatccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 11 - DNA, 5455 bp, synthetic construct
  source 1..5455: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcgagctcggtacctcgcgaatacatctagatatcggatccactagtcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccggtccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 12 - DNA, 6275 bp, synthetic construct
  source 1..6275: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcgagctcggtacctcgcgaatacatctagatatcggatccactagtcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgtccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 13 - DNA, 6340 bp, synthetic construct
  source 1..6340: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcgagctcggtacctcgcgaatacatctagatatcggatccactagtggtaccaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccatccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 7901 bp, synthetic construct
  source 1..7901: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcgagctcggtacctcgcgaatacatctagatatcggatccactagtcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccggtccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 1405 bp, synthetic construct
  source 1..1405: mol_type = other DNA; note = gfa1405 Promoter Sequence
actagtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgtccgga

SEQ ID NO: 16 - DNA, 4369 bp, synthetic construct
  source 1..4369: mol_type = other DNA
gcggccgcatcgattgaattcgagctcggtacctcgcgaatacatctagatatcggatccactagtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgtccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgc

SEQ ID NO: 17 - DNA, 4640 bp, synthetic construct
  source 1..4640: mol_type = other DNA; note = gfa1405 with various other elements and with 5 prime and 3 prime ITRs
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcgagctcggtacctcgcgaatacatctagatatcggatccactagtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgtccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 7086 bp, synthetic construct
  source 1..7086: mol_type = other DNA; note = AAV9.gfa1405.eiF2B5
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcgagctcggtacctcgcgaatacatctagatatcggatccactagtaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgtccggaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccaattctgcagtcgacggtaccgcgggcccgggatccaccggttcgccaccatggcggcccctgtagtggcgccgcctggtgtggtggttagtcgggctaacaagcgcagcggcgcggggccgggaggcagcggtggcgggggagccagaggggcggaggaggaaccgccgccgcccctacaagcagttctggtggccgatagcttcgatcgccgcttcttccccatctccaaggaccagcctcgggtcctcttgcccctggccaatgtggcattaattgactacactctggaattcctgactgccacaggtgtacaggaaacatttgtcttttgttgctggaaagctgctcaaatcaaagaacatttactgaagtcaaagtggtgccgccctacatctctcaatgtggttcgaataattacatcagagctctatcgatcactgggagatgtcctccgtgatgttgatgccaaggctttggtgcgctctgactttcttctggtgtatggggatgtcatctcaaacatcaatatcaccagagcccttgaggaacacaggttgagacggaagctagaaaaaaatgtttctgtgatgacgatgatcttcaaggagtcatcccccagccacccaactcgttgccacgaagacaatgtggtagtggctgtggatagtaccacaaacagggttctccattttcagaagacccagggtctccggcgttttgcatttcctctgagcctgtttcagggcagtagtgatggagtggaggttcgatatgatttactggattgtcatatcagcatctgttctcctcaggtggcacaactctttacagacaactttgactaccaaactcgagatgactttgtgcgaggtctcttagtgaatgaggagatcctagggaaccagatccacatgcacgtaacagctaaggaatatggtgcccgtgtctccaacctacacatgtactcagctgtctgtgctgacgtcatccgccgatgggtctaccctctcaccccagaggcgaacttcactgacagcaccacccagagctgcactcattcccggcacaacatctaccgagggcctgaggtcagcctgggccatggcagcatcctagaggaaaatgtgctcctgggctctggcactgtcattggcagcaattgctttatcaccaacagtgtcattggccccggctgccacattggtgataacgtggtgctggaccagacctacctgtggcagggtgttcgagtggcggctggagcacagatccatcagtctctgctttgtgacaatgctgaggtcaaggaacgagtgacactgaaaccacgctctgtcctcacttcccaggtggtcgtgggcccaaatatcacgctgcctgagggctcggtgatctctttgcaccctccagatgcagaggaagatgaagatgatggcgagttcagtgatgattctggggctgaccaagaaaaggacaaagtgaagatgaaaggttacaatccagcagaagtaggagctgctggcaagggctacctctggaaagctgcaggcatgaacatggaggaagaggaggaactgcagcagaatctgtggggactcaagatcaacatggaagaagagagtgaaagtgaaagtgagcaaagtatggattctgaggagccggacagccggggaggctcccctcagatggatgacatcaaagtgttccagaatgaagttttaggaacactacagcggggcaaagaggagaacatttcttgtgacaatctcgtcctggaaatcaactctctcaagtatgcctataacataagtctaaaggaggtgatgcaggtactgagccacgtggtcctggagttccccctgcaacagatggattccccgcttgactcaagccgctactgtgccctgctgcttcctctgctaaaggcctggagccctgtttttaggaactacataaagcgcgcagccgaccatttggaagcgttagcagccattgaggacttcttcctagagcatgaagctcttggtatttccatggccaaggtactgatggctttctaccagctggagatcctggctgaggaaacaattctgagctggttcagccaaagagatacaactgacaagggccagcagttgcgcaagaatcaacagctgcagaggttcatccagtggctaaaagaggcagaagaggagtcatctgaagatgactgacgcgcgcgagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt